(19) United States
(12) Patent Application Publication
(10) Pub. No.: US 2022/0071684 A1
(43) Pub. Date: Mar. 10, 2022

(54) DEVICES, SYSTEMS AND METHODS FOR SUBDERMAL COAGULATION

(71) Applicant: Apyx Medical Corporation, Clearwater, FL (US)

(72) Inventors: Gary G. Gogolin, Tampa, FL (US); Shawn D. Roman, Safety Harbor, FL (US); Fredrik Jonsson, St. Petersburg, FL (US); Gregory Goliszek, Oldsmar, FL (US); Brian S. Gerahart, Treasure Island, FL (US); Ahmed H. Arikat, Clearwater, FL (US); Bradley A. Rentschler, New Port Richley, FL (US)

(21) Appl. No.: 17/312,984

(22) PCT Filed: Dec. 19, 2019
(86) PCT No.: PCT/US2019/067413

§ 371 Date: Jun. 11, 2021

Related U.S. Application Data
(60) Provisional application No. 62/782,012, filed on Dec. 19, 2018.

(51) Int. Cl. A61B 18/04

(52) U.S. Cl. CPC A61B 18/042; A61B 2018/00458

(57) ABSTRACT

Devices, systems and methods are provided for subdermal tissue tightening through soft tissue coagulation and for use in cosmetic surgery applications. The devices, systems and methods of the present disclosure may be used for a minimally invasive application of helium-based cold plasma energy to subcutaneous tissue for the purpose of tightening lax tissue. In various aspects of the present disclosure, distal tips, each including at least one port for applying plasma to patient tissue are provided for use with an electrosurgical apparatus.

### PRIORITY

This application claims priority to U.S. Provisional Patent Application No. 62/782,012, filed Dec. 19, 2018, entitled “DEVICES, SYSTEMS AND METHODS FOR SUBDERMAL COAGULATION”, the contents of which are hereby incorporated by reference in its entirety.

### BACKGROUND

### Field

The present disclosure relates generally to electrosurgery and electrosurgical systems and apparatuses, and more particularly, to electrosurgical devices, systems and methods for subdermal tissue tightening through soft tissue coagulation and for use in cosmetic surgery applications.

### Description of the Related Art

High frequency electrical energy has been widely used in surgery and is commonly referred to as electrosurgical energy. Tissue is cut and bodily fluids are coagulated using electrosurgical energy.

Gas plasma is an ionized gas capable of conducting electrical energy. Plasmas are used in surgical devices to conduct electrosurgical energy to a patient. The plasma conducts the energy by providing a pathway of relatively low electrical resistance. The electrosurgical energy will follow through the plasma to cut, coagulate, desiccate, or fulgurate blood or tissue of the patient. There is no physical contact required between an electrode and the tissue treated.

Electrosurgical systems that do not incorporate a source of regulated gas can ionize the ambient air between the active electrode and the patient. The plasma that is thereby created will conduct the electrosurgical energy to the patient, although the plasma arc will typically appear more spatially dispersed compared with systems that have a regulated flow of ionizable gas.

Atmospheric pressure discharge cold plasma applicators have found use in a variety of applications including surface sterilization, hemostasis, and ablation of tumors.

Often, a simple surgical knife is used to excise the tissue in question, followed by the use of a cold plasma applicator for cauterization, sterilization, and hemostasis. Cold plasma beam applicators have been developed for both open and endoscopic procedures. In the latter case, it is often desirable to be able to redirect the position of the cold plasma beam tip to a specific operative site. The external incision and pathway for the endoscopic tool may be chosen to avoid major blood vessels and non-target organs and may not coincide with an optimum alignment for the target internal tissue site. A means of redirecting the cold plasma beam is essential in these situations.

The heat effects of the radiofrequency (RF) alternating current used in electrosurgery on cells and tissue have been well established. Normal body temperature is 37° C. and, with normal illness, can increase to 40° C. without permanent impact or damage to the cells of our body. However, when the temperature of cells in tissue reaches 50° C., cell death occurs in approximately 6 minutes. When the temperature of cells in tissue reaches 60° C., cell death occurs instantaneously. Between the temperatures of 60° C. and just below 100° C., two simultaneous processes occur. The first is protein denaturation leading to coagulation which will be discussed in more detail below. The second is desiccation or dehydration as the cells lose water through the thermally damaged cellular wall. As temperatures rise above 100° C., intracellular water turns to steam and tissue cells begin to vaporize as a result of the massive intracellular expansion that occurs. Finally, at temperatures of 200° C. or more, organic molecules are broken down into a process called carbonization. This leaves behind carbon molecules that give a black and/or brown appearance to the tissue.

Understanding these heat effects of RF energy on cells and tissue can allow the predictable changes to be used to accomplish beneficial therapeutic results. Protein denaturation leading to soft tissue coagulation is one of the most versatile and widely utilized tissue effects. Protein denaturation is the process in which hydrothermal bonds (crosslinks) between protein molecules, such as collagen, are instantaneously broken and then quickly reformed as tissue cools. This process leads to the formation of uniform clumps of protein typically called coagulum through a subsequent process known as coagulation. In the process of coagulation, cellular proteins are altered but not destroyed and form protein bonds that create homogenous, gelatinous structures. The resulting tissue effect of coagulation is extremely useful and most commonly used for occluding blood vessels and causing hemostasis.

In addition to causing hemostasis, coagulation results in predictable contraction of soft tissue. Collagen is one of the main proteins found in human skin and connective tissue. The coagulation/denaturation temperature of collagen is conventionally stated to be 66.8° C., although this can vary for different tissue types. Once denatured, collagen rapidly contracts as fibers shrink to one-third of their overall length. However, the amount of contraction is dependent upon the temperature and the duration of the treatment. The hotter the temperature the shorter amount of treatment time needed for maximal contraction. For example, collagen heated at a temperature of 65° C. must be heated for greater than 120 seconds for significant contraction to occur.

Thermal-induced contraction of collagen through the coagulation of soft tissue is well known in medicine and is used in ophthalmology, orthopedic applications, and the treatment of varicose veins. The reported range of temperatures causing collagen contraction varies from 60° C. to 85° C. Therefore, once tissue is heated to within this range, protein denaturation and collagen contraction occur resulting in the reduction in volume and surface area of the heated tissue. Noninvasive radiofrequency devices, lasers, and plasma devices have been used for the reduction of facial wrinkles and rhytides caused by thermal-induced collagen/tissue contraction since the mid-1990s.

### SUMMARY

The present disclosure relates to devices, systems and methods for subdermal tissue tightening through soft tissue coagulation and for use in cosmetic surgery applications. The devices, systems and methods of the present disclosure may be used for a minimally invasive application of plasma energy to subcutaneous tissue for the purpose of tightening lax tissue.

In one aspect of the present disclosure, an electrosurgical apparatus is provided comprising: a housing; a shaft extending from the housing and disposed along a longitudinal axis; an electrically conducting member; a distal tip including an interior, an outer wall, and at least one port, the at least one port disposed through the outer wall and oriented in a radial direction relative to the longitudinal axis, the electrically conducting member at least partially disposed in the interior of the distal tip and configured to energize inert gas provided via the shaft to the interior of the distal tip such that plasma is ejected from the at least one port.

In another aspect, the electrosurgical apparatus is provided, wherein the at least one port is configured such that the distal tip has a 180-degree tissue treatment area about the longitudinal axis.

In another aspect, the electrosurgical apparatus is provided, wherein the interior of the distal tip includes an inner wall that is slanted with respect to the longitudinal axis and is configured to direct the plasma generated by the electrosurgical apparatus and the inert gas provided to the distal tip through the at least one port to the exterior of the electrosurgical apparatus.

In another aspect, the electrosurgical apparatus is provided, wherein the distal tip includes at least one second port disposed through the outer wall of the distal tip and oriented in a radial direction to the longitudinal axis, the at least one second port diametrically opposed from the at least one first port.

In another aspect, the electrosurgical apparatus is provided, wherein the interior of the distal tip includes an inner wall having a first portion and a second portion, the first portion is slanted with respect to the longitudinal axis and is configured to direct the plasma generated by the electrosurgical apparatus and the inert gas provided to the distal tip through the at least one first port to the exterior of the electrosurgical apparatus, the second portion is slanted with respect to the longitudinal axis and is configured to direct the plasma generated by the electrosurgical apparatus and the inert gas provided to the distal tip through the at least one second portion to the exterior of the electrosurgical apparatus.

In another aspect, the electrosurgical apparatus is provided, wherein the at least one first port and at least one second port are configured such that the distal tip has a 360-degree tissue treatment area about the longitudinal axis.

In another aspect, the electrosurgical apparatus is provided, comprising a support tube having a proximal and a distal end, wherein the proximal end of the support tube is disposed through a distal end of the shaft and coupled to the interior of the shaft and the distal end of the support tube is disposed through a proximal end of the distal tip and coupled to the interior of the distal tip, the support tube configured to couple the distal tip to the distal end of the shaft and to provide support to the coupling of the distal tip to the distal end of the shaft.

In another aspect, the electrosurgical apparatus is provided, wherein the support tube is made of a non-conducting material.

In another aspect, the electrosurgical apparatus is provided, wherein the support tube is coupled the shaft and distal tip via an adhesive.

In another aspect, the electrosurgical apparatus is provided, wherein the electrically conducting member is a support tube having a proximal and a distal end, wherein the proximal end of the support tube is disposed through a distal end of the shaft and coupled to the interior of the shaft and the distal end of the support tube is disposed through a proximal end of the distal tip and coupled to the interior of the distal tip, the support tube configured couple the distal tip to the distal end of the shaft and to provide support to the coupling of the distal tip to the distal end of the shaft.

In another aspect, the electrosurgical apparatus is provided, further comprising a coupling member disposed between the shaft and the distal tip, the coupling member configured to couple the distal tip to the shaft.

In another aspect, the electrosurgical apparatus is provided, further comprising a support tube having a proximal and a distal end, wherein the proximal end of the support tube is disposed through a distal end of the shaft and coupled to the interior of the shaft, the distal end of the support tube is disposed through a proximal end of the distal tip and coupled to the interior of the distal tip, and the coupling member is formed via injection molding between the distal end of the shaft and the proximal end of the distal tip over the support tube.

In another aspect, the electrosurgical apparatus is provided, wherein the support tube is coupled the shaft and distal tip via an adhesive.

In another aspect, the electrosurgical apparatus is provided, wherein the interior of the distal tip includes a slot that receives a distal end of the electrically conducting member.

In another aspect, the electrosurgical apparatus is provided, wherein the electrically conducting member includes a bent distal end disposed in the slot, the bent distal end configured to prevent distal tip from being decoupled from the shaft.

In another aspect, the electrosurgical apparatus is provided, wherein the distal tip includes a cap that is formed via injection molding over a distal end of the electrically conducting member to prevent the distal tip from being decoupled from the shaft.

In another aspect, the electrosurgical apparatus is provided, wherein the distal tip is formed via injection molding over a distal end of the electrically conducting member to prevent the distal tip from being decoupled from the shaft.

In another aspect, the electrosurgical apparatus is provided, wherein the distal tip includes at least one protrusion and a distal end of the shaft includes at least one slot configured to receive the protrusion such that the distal tip is securely coupled to the distal end of the shaft.

In another aspect, the electrosurgical apparatus is provided, wherein the at least one slot includes a first portion aligned along the longitudinal axis and a second portion extending perpendicularly to the longitudinal axis.

In another aspect, the electrosurgical apparatus is provided, further comprising a connector and a cable having a first end and a second end, the first end of the cable coupled to the housing and the second end of the cable coupled to the connector, the connector configured to be coupled to an electrosurgical generator to receive electrosurgical energy and the inert gas to be provided to the housing via the cable.

In another aspect, the electrosurgical apparatus is provided, further comprising a stranded wire that couples the electrically conducting member to the cable, the stranded wire is configured to provide electrosurgical energy to the electrically conducting member.

In another aspect, the electrosurgical apparatus is provided, wherein the shaft includes at least one marking disposed a predetermined distance from one of a distal end of the distal tip or a center of the at least one port, such that when the at least one marking becomes visible to a user as the distal tip and shaft are pulled from patient tissue, the user is alerted to deactivate the electrosurgical apparatus.

In another aspect of the present disclosure, a method for using a plasma device to tighten tissue is provided, the method comprising: creating an incision through tissue to access a subdermal tissue plane; inserting the plasma device into the subdermal tissue plane; activating the plasma device to generate and apply plasma to the subdermal tissue plane; moving the plasma device through the subdermal tissue plane; and heating tissue in the subdermal tissue plane to a predetermined temperature to tighten the tissue.

In another aspect, the method is provided, wherein a waveform including a predetermined power curve is applied to an electrode of the plasma device when the plasma device is activated.

In another aspect, the method is provided, wherein the predetermined power curve is configured such that the power applied to the electrode is between 24 and 32 Watts.

In another aspect, the method is provided, wherein the predetermined power curve is configured such that the generated plasma is pulsed.

In another aspect, the method is provided, wherein each pulse of the pulsed plasma includes a predetermined time duration.

In another aspect, the method is provided, wherein the predetermined time duration is between 0.04 and 0.08 seconds.

In another aspect, the method is provided, wherein inert gas is provided at a predetermined flow rate when the plasma device is activated.

In another aspect, the method is provided, wherein the predetermined flow rate is between 1.5 liters per minute to 3 liters per minute.

In another aspect, the method is provided, wherein the inert gas is helium.

In another aspect, the method is provided, wherein the predetermined temperature is approximately 85 Celsius.

In another aspect, the method is provided, wherein a distal tip of the plasma device is moved through the subdermal tissue plane at a predetermined speed.

In another aspect, the method is provided, wherein the predetermined speed is 1 centimeter per second.

In another aspect, the method is provided, further comprising: removing the plasma device from the subdermal tissue plane; and closing the entry incision.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is an illustration of an exemplary electrosurgical system in accordance with an embodiment of the present disclosure;

FIG. 2A is a schematic diagram showing a side view of an electrosurgical apparatus in accordance with an embodiment of the present disclosure;

FIG. 2B is a front view of the electrosurgical apparatus shown in FIG. 2A:

FIG. 2C is a cross sectional view of the electrosurgical apparatus shown in FIG. 2A taken along line A-A;

FIG. 3A is an enlarged cross-sectional view of the electrosurgical apparatus in accordance with an embodiment of the present disclosure;

FIG. 3B illustrates a front view of the electrosurgical apparatus shown in FIG. 3A taken along line B-B;

FIG. 4 is an enlarged cross-sectional view of the electrosurgical apparatus shown in FIG. 3A with a blade extended;

FIG. 5 illustrates an exemplary electrosurgical apparatus including an articulating distal end in accordance with an embodiment of the present disclosure;

FIG. 6 is a perspective view of an electrosurgical apparatus in accordance with another embodiment of the present disclosure;

FIG. 7 is a cross-sectional view of the anatomy of human cutaneous tissue;

FIG. 8 is a flowchart illustrating an exemplary method for tightening tissue in accordance with an embodiment of the present disclosure;

FIG. 9A is a perspective view of an electrosurgical apparatus in accordance with another embodiment of the present disclosure;

FIGS. 9B-9F include various views of a distal tip of the electrosurgical apparatus of FIG. 9A in accordance with an embodiment of the present disclosure;

FIGS. 10A-10G include various views of a distal tip for use with the electrosurgical apparatus of FIG. 9A in accordance with another embodiment of the present disclosure;

FIGS. 11A and 11B include various views of a distal tip for use with the electrosurgical apparatus of FIG. 9A in accordance with another embodiment of the present disclosure;

FIGS. 11C and 11 D include various views of a distal tip for use with the electrosurgical apparatus of FIG. 9A in accordance with another embodiment of the present disclosure;

FIGS. 11E and 11F include various views of a distal tip for use with the electrosurgical apparatus of FIG. 9A in accordance with another embodiment of the present disclosure;

FIGS. 11G and 11H include various views of a distal tip for use with the electrosurgical apparatus of FIG. 9A in accordance with another embodiment of the present disclosure;

FIGS. 11I and 11J include various views of a distal tip for use with the electrosurgical apparatus of FIG. 9A in accordance with another embodiment of the present disclosure;

FIGS. 11K, 11L, 11M, 11N include various views of a distal tip for use with electrosurgical apparatus of FIG. 9A in accordance with another embodiment of the present disclosure;

FIG. 12A is a perspective view of an electrosurgical apparatus in accordance with another embodiment of the present disclosure;

FIGS. 12B-12E include side cross-sectional views of various components of the electrosurgical apparatus of FIG. 12A in accordance with an embodiment of the present disclosure;

FIG. 12F is a perspective view of the distal tip and a tip protector of the electrosurgical apparatus of FIG. 12A in accordance with an embodiment of the present disclosure;

FIG. 12G shows the distal tip of the electrosurgical apparatus of FIG. 12A inserted through a tissue surface into a subdermal plane in accordance with an embodiment of the present disclosure;

FIG. 12H is a front view of a trace card for use with the electrosurgical apparatus of FIG. 12A in accordance with another embodiment of the present disclosure;

FIGS. 12I, 12J, 12K are perspective views of a distal tip and the distal portion of a shaft of an electrosurgical apparatus in accordance with another embodiment of the present disclosure;

FIGS. 12L, 12M include various views of a distal tip and the distal portion of a shaft of an electrosurgical apparatus in accordance with another embodiment of the present disclosure;

FIGS. 12N, 12O include various views of a distal tip and the distal portion of a shaft of an electrosurgical apparatus in accordance with another embodiment of the present disclosure;

FIG. 12P includes a partial cross-sectional view of a protrusion of the distal tip of

FIGS. 12N, 12O in accordance with an embodiment of the present disclosure;

FIGS. 12Q, 12R include various views of a distal tip and the distal portion of a shaft of an electrosurgical apparatus in accordance with another embodiment of the present disclosure;

FIGS. 12S, 12T include various views of a distal tip and the distal portion of a shaft of an electrosurgical apparatus in accordance with another embodiment of the present disclosure;

FIG. 13A is a perspective view of a distal tip of an electrosurgical apparatus with a cap of the distal tip shown in phantom in accordance with an embodiment of the present disclosure;

FIG. 13B is a perspective view of the distal tip of FIG. 13A with the cap and a tubular portion of the distal tip of FIG. 13A shown in phantom in accordance with the present disclosure;

FIG. 13C is a side-perspective view of the tubular portion of the distal tip of FIG. 13A in accordance with the present disclosure;

FIG. 13D is a perspective view of an electrode for use with the distal tip of FIG. 13A in accordance with an embodiment of the present disclosure;

FIG. 14A is a side perspective view of a distal tip of an electrosurgical apparatus in accordance with an embodiment of the present disclosure;

FIG. 14B is the side perspective view of FIG. 14A with a cap of the distal tip of FIG. 14A shown in phantom in accordance with the present disclosure;

FIG. 14C is the side perspective view of FIG. 14A with the cap and a tubular portion of the distal tip of FIG. 14A shown in phantom in accordance the present disclosure;

FIG. 14D is a perspective view of the tubular portion of the distal tip of FIG. 14A in accordance with the present disclosure;

FIG. 14E is a side cross-section view of the cap of the distal tip of FIG. 14A in accordance with the present disclosure;

FIG. 15A is a side perspective view of a distal tip of an electrosurgical apparatus in accordance with an embodiment of the present disclosure;

FIG. 15B is the side perspective view of FIG. 15A with a cap of the distal tip of FIG. 15A shown in phantom in accordance with the present disclosure;

FIG. 15C is the side perspective view of FIG. 15A with the cap and a tubular portion of the distal tip of FIG. 15A shown in phantom in accordance the present disclosure;

FIG. 15D is a perspective view of the tubular portion of the distal tip of FIG. 15A in accordance with the present disclosure;

FIG. 15E is a perspective view of an electrode for use with the distal tip of FIG. 14A in accordance with an embodiment of the present disclosure;

FIG. 16A is a side perspective view of a distal tip of an electrosurgical apparatus in accordance with an embodiment of the present disclosure;

FIG. 16B is the side perspective view of FIG. 16A with a cap of the distal tip of FIG. 16A shown in phantom in accordance with the present disclosure;

FIG. 16C is the side perspective view of FIG. 16A with the cap and a tubular portion of the distal tip of FIG. 16A shown in phantom in accordance the present disclosure;

FIG. 16D is a perspective view of the tubular portion of the distal tip of FIG. 16A in accordance with the present disclosure;

FIG. 16E is a perspective view of an electrode for use with the distal tip of FIG.

16A in accordance with an embodiment of the present disclosure;

FIG. 17A is a perspective view of a distal tip of an electrosurgical apparatus in accordance with an embodiment of the present disclosure;

FIGS. 17B is the side view of FIG. 17A with the distal tip of FIG. 17A shown in phantom in accordance with the present disclosure;

FIGS. 17C is a perspective view of the distal tip of FIG. 17A with the distal tip of FIG. 16A shown in phantom in accordance with the present disclosure;

FIG. 18A is a side view of a distal tip of an electrosurgical apparatus in accordance with an embodiment of the present disclosure;

FIG. 18B is a cross-section view of the distal tip of FIG. 18A in accordance with the present disclosure;

FIG. 19A is a side view of a distal tip of an electrosurgical apparatus in accordance with an embodiment of the present disclosure;

FIG. 19B is another side view of the distal tip of FIG. 19A in accordance with an embodiment of the present disclosure;

FIG. 19C is a side, perspective, cross-section view of the distal tip of FIG. 19A in accordance with the present disclosure;

FIG. 19D is a view through the proximal end of the distal tip of FIG. 19A in accordance with the present disclosure;

FIG. 19E is a perspective view of an electrode for use with the distal tip of FIG. 19A in accordance with the present disclosure;

FIG. 19F is another side view of the distal tip of FIG. 19A in accordance with the present disclosure;

FIG. 20A is a side view of a distal tip of an electrosurgical apparatus in accordance with an embodiment of the present disclosure;

FIG. 20B is a side, perspective, cross-section view of the distal tip of FIG. 20A in accordance with the present disclosure;

FIG. 20C is another side view of the distal tip of FIG. 20A in accordance with the present disclosure;

FIG. 20D is a view through the proximal end of the distal tip of FIG. 20A in accordance with the present disclosure;

FIG. 20E is a perspective view of an electrode for use with the distal tip of FIG. 20A in accordance with the present disclosure;

FIG. 21A is a side perspective view of a distal tip of an electrosurgical apparatus in accordance with an embodiment of the present disclosure;

FIG. 21B is a side, perspective, cross-section view of the distal tip of FIG. 21A in accordance with the present disclosure;

FIG. 21C is another side perspective view of the distal tip of FIG. 21A in accordance with the present disclosure;

FIGS. 21D is a view of the distal end of the distal tip of FIG. 21A in accordance with the present disclosure;

FIG. 22A is a side view comparison of the distal tip of FIG. 21A and another distal tip in accordance with an embodiment of the present disclosure;

FIG. 22B is a view of the distal end comparing the distal tips of FIG. 22B in accordance with the present disclosure;

FIG. 23 shows an effective treatment area of several of the electrosurgical apparatuses of the present disclosure;

FIG. 24 is a flowchart illustrating an exemplary method for tightening tissue in accordance with an embodiment of the present disclosure;

FIG. 25 is a graph comparing the thermal effects on tissue caused by various devices; and

FIG. 26 illustrates power versus impedance curves for various devices.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., instrument, apparatus, applicator, handpiece, forceps, etc., which is closer to the user, while the term “distal” will refer to the end which is further from the user. Herein, the phrase “coupled” is defined to mean directly connected to or indirectly connected with through one or more intermediate components. Such intermediate components may include both hardware and software based components.

Recently, the use of thermal-induced collagen/tissue contraction has been expanded to minimally invasive procedures. Laser-assisted lipolysis (LAL) and radiofrequency-assisted lipolysis (RFAL) devices have combined the removal of subcutaneous fat with soft tissue heating to reduce the skin laxity that often results from fat volume removal. These devices are placed in the same subcutaneous tissue plane as a standard suction-assisted lipolysis (SAL) cannula and are used to deliver thermal energy to coagulate the subcutaneous tissue including the underside of the dermis, the fascia, and the septal connective tissue. The coagulation of the subcutaneous tissue results in collagen/tissue contraction that reduces skin laxity.

The devices, systems and methods of the present disclosure are employed for the minimally invasive application of helium-based cold plasma energy to subcutaneous tissue for the purpose of tightening lax tissue. A tip of a plasma generating handpiece is placed in the subcutaneous tissue plane through the same access ports used for SAL. Activation of the plasma generating handpiece in this plane causes contraction of the collagen contained in the dermis, the fascia, and the septal connective matrix through precise heating from the plasma energy.

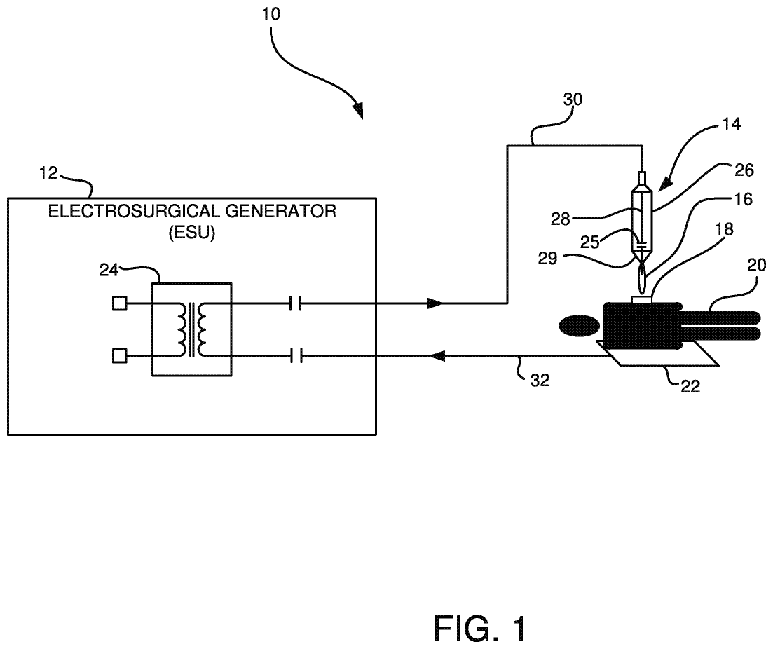

FIG. 1 shows an exemplary electrosurgical system generally indicated as 10 comprising an electrosurgical generator (ESU) generally indicated as 12 to generate power for the electrosurgical apparatus 10 and a plasma generator generally indicated as 14 to generate and apply a plasma stream 16 to a surgical site or target area 18 on a patient 20 resting on a conductive plate or support surface 22. The electrosurgical generator 12 includes a transformer generally indicated as 24 including a primary and secondary coupled to an electrical source (not shown) to provide high frequency electrical energy to the plasma generator 14. Typically, the electrosurgical generator 12 comprises an isolated floating potential not referenced to any potential. Thus, current flows between the active and return electrodes. If the output is not isolated, but referenced to “earth”, current can flow to areas with ground potential. If the contact surface of these areas and the patient is relatively small, an undesirable burning can occur.

The plasma generator 14 comprises a handpiece or holder 26 having an electrode 28 at least partially disposed within a fluid flow housing 29 and coupled to the transformer 24 to receive the high frequency electrical energy therefrom to at least partially ionize noble gas fed to the fluid flow housing 29 of the handpiece or holder 26 to generate or create the plasma stream 16. The high frequency electrical energy is fed from the secondary of the transformer 24 through an active conductor 30 to the electrode 28 (collectively active electrode) in the handpiece 26 to create the plasma stream 16 for application to the surgical site 18 on the patient 20. Furthermore, in one embodiment, a current limiting capacitor 25 is provided in series with the electrode 28 to limit the amount of current being delivered to the patient 20.

The return path to the electrosurgical generator 12 is through the tissue and body fluid of the patient 20, the conductor plate or support member 22 and a return conductor 32 (collectively return electrode) to the secondary of the transformer 24 to complete the isolated, floating potential circuit.

In another embodiment, the electrosurgical generator 12 comprises an isolated non-floating potential not referenced to any potential. The plasma current flow back to the electrosurgical generator 12 is through the tissue and body fluid and the patient 20. From there, the return current circuit is completed through the combined external capacitance to the plasma generator handpiece 26, surgeon and through displacement current. The capacitance is determined, among other things, by the physical size of the patient 20. Such an electrosurgical apparatus and generator are described in commonly owned U.S. Pat. No. 7,316,682 to Konesky, the contents of which are hereby incorporated by reference in its entirety.

It is to be appreciated that transformer 24 may be disposed in the plasma generator handpiece 26, as will be described in various embodiments below. In this configuration, other transformers may be provided in the generator 12 for providing a proper voltage and current to the transformer in the handpiece 26, e.g., a step-down transformer, a step-up transformer or any combination thereof. Alternatively, the transformer may be located in the generator.

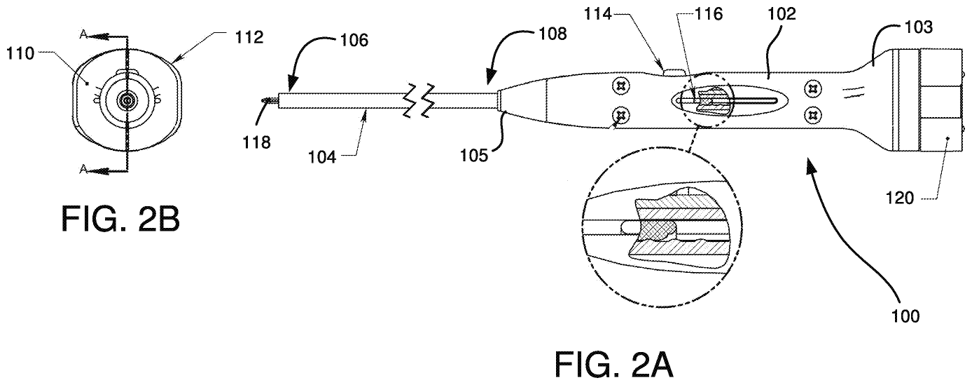

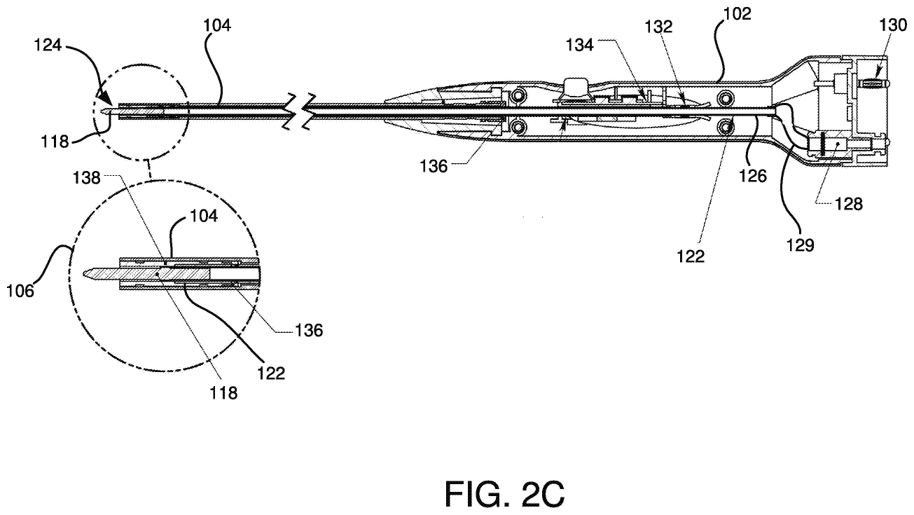

Referring to FIGS. 2A-2C, an electrosurgical handpiece or plasma generator 100 in accordance with the present disclosure is illustrated. Generally, the handpiece 100 includes a housing 102 having a proximal end 103 and a distal end 105 and a tube 104 having an open distal end 106 and a proximal end 108 coupled to the distal end 105 of the housing 102. The housing 102 includes a right side housing 110 and left side housing 112, and further includes provisions for a button 114 and slider 116. Activation of the slider 116 will expose an optional blade 118 at the open distal end 106 of the tube 104. Activation of the button 114 will apply electrosurgical energy to the blade 118 and, in certain embodiments, enable gas flow through the flow tube 122, as will be described in detail below.

Additionally, a transformer 120 may be provided on the proximal end 103 of the housing 102 for coupling a source of radio frequency (RF) energy to the handpiece 100. By providing the transformer 120 in the handpiece 100 (as opposed to locating the transformer in the electrosurgical generator), power for the handpiece 100 develops from higher voltage and lower current than that required when the transformer is located remotely in the generator, which results in lower thermalization effects. In contrast, a transformer back in the generator produces applicator power at a lower voltage, higher current with greater thermalization effects. Therefore, by providing the transformer 120 in handpiece 100, collateral damage to tissue at the operative site is minimized. While providing the transformer in the handle has advantages, it is contemplated that the transformer may be disposed in the generator.

A cross section view along line A-A of the housing 102 is shown in FIG. 2C. Disposed within the housing 102 and tube 104 is flow tube 122 which runs along the longitudinal axis of the handpiece or plasma generator 100. On a distal end 124 of the flow tube 122, the blade 118 is retained within the flow tube 122. A proximal end 126 of the flow tube 122 is coupled to a source of gas via a tube connector 128 and flexible tubing 129. The proximal end 126 of the flow tube 122 is also coupled to a source of RF energy via plug 130 which couples to transformer 120. The flow tube 122 is made of an electrically conducting material, preferably stainless steel, as to conduct the RF energy to the blade 118 when being employed for plasma applications or electrosurgical cutting as will be described below. The outer tube 104 is constructed from non-conductive material, e.g., Lestran™. The slider 116 is coupled to the flow tube 122 via a retaining collar 132. A printed circuit board (PCB) 134 is disposed in the housing 102 and controls the application of the RF energy from the transformer 120 via the button 114.

It is to be appreciated that the slider 116 may be freely moveable in a linear direction or may include a mechanism for incremental movements, e.g., a ratchet movement, to prevent an operator of the handpiece 100 from over extending the blade 118. By employing a mechanism for incremental movements of the optional blade 118, the operator will have greater control over the length of the exposed blade 118 to avoid damage to tissue at the surgical site. It is also contemplated that the slider may extend a needle or blunt probe instead of a blade, with extension or retraction of the blade/needle/probe helping to control the characteristics of the energy transfer to the gas and, in combination with gas flow, the beam shape and intensity.

An enlarged view of the distal end 106 of the outer tube 104 is also illustrated in FIG. 2C. Here, the blade 118 is coupled to the flow tube 122 which is held in place in the outer tube 104 by at least one seal 136. The at least one seal 136 prevents backflow of gas into tube 104 and housing 102. A cylindrical ceramic insert 138 is disposed in the distal end of the outer tube 104 to maintain the blade along the longitudinal axis of the handpiece 100 and provide structural support during mechanical cutting when the blade is exposed beyond the distal end of the outer tube 104.

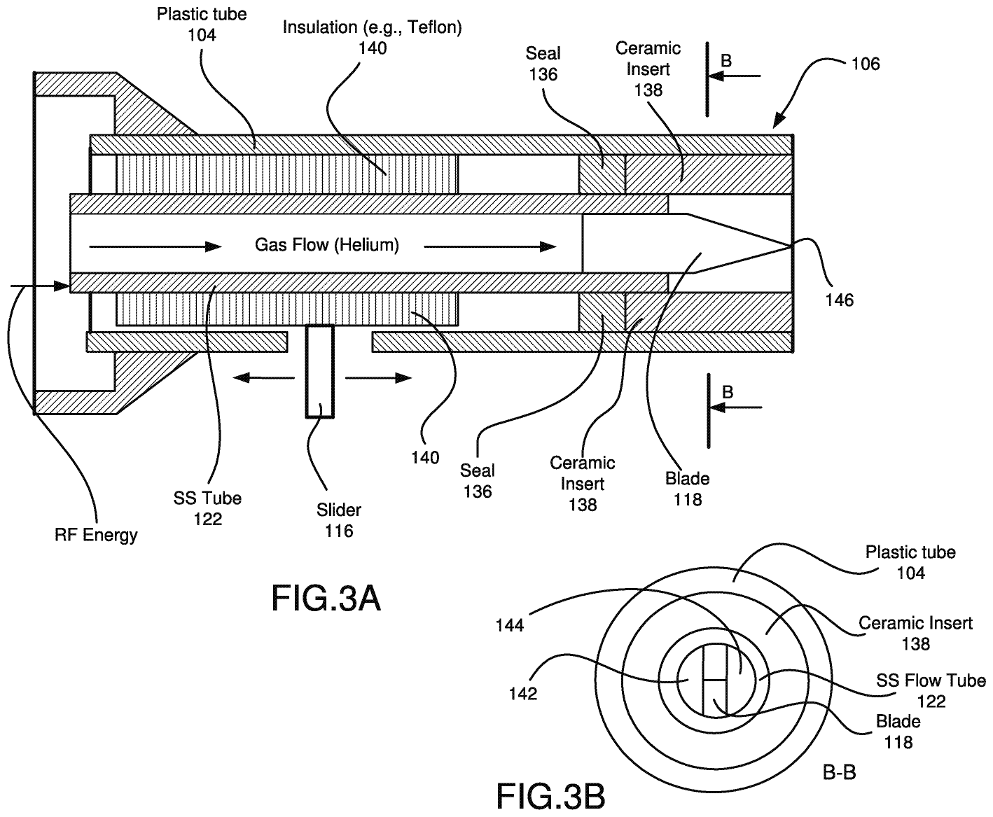

The operational aspect of the handpiece 100 will now be described in relation to FIGS. 3A and 3B, where FIG. 3A shows an enlarged cross section of the apparatus and FIG. 3B illustrates a front view of the apparatus.

Referring to FIG. 3A, the flow tube 122 is disposed in the outer tube 104 with a cylindrical insulator 140 disposed around the flow tube 122. Slider 116 is coupled to the insulator 140 and is employed to extend and retract the blade 118. At the distal end 106 of the outer tube 104, the annular or ring-shaped seal 136 and cylindrical ceramic insert 138 are disposed about the flow tube 122. As can be seen In FIG. 3B, the generally planar blade 118 is coupled to an inner circumference of the cylindrical flow tube 122 such that two gas passageways 142, 144 are formed on the both sides of the blade 118. As gas flows from the proximal end 103 of the housing through the flow tube 122, the gas will pass over the blade 118 out the distal end of the outer tube 104.

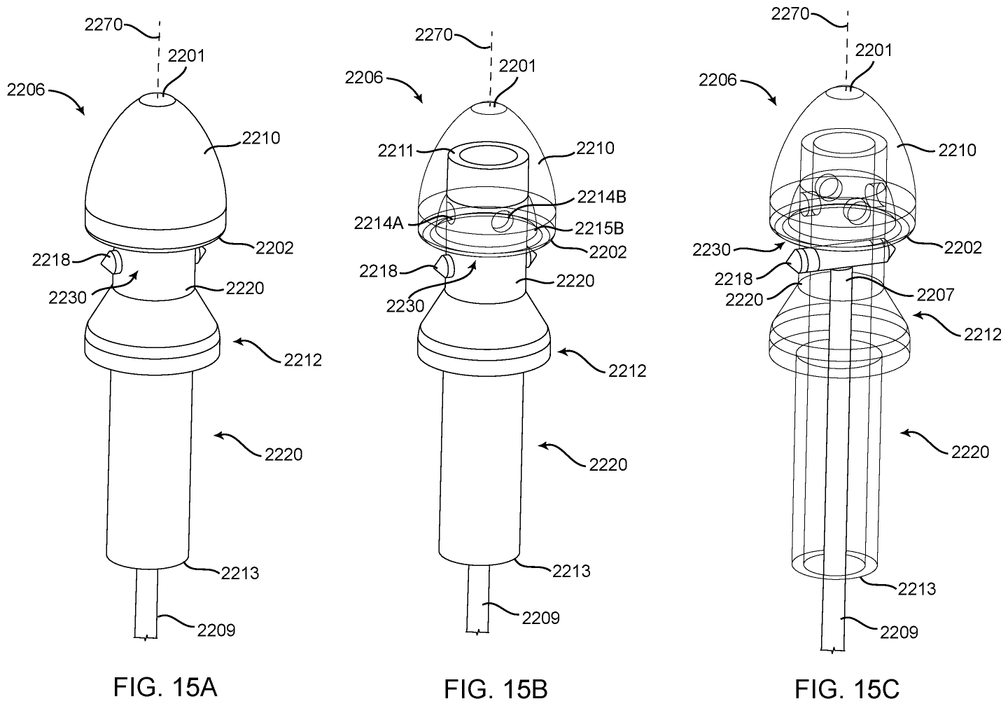

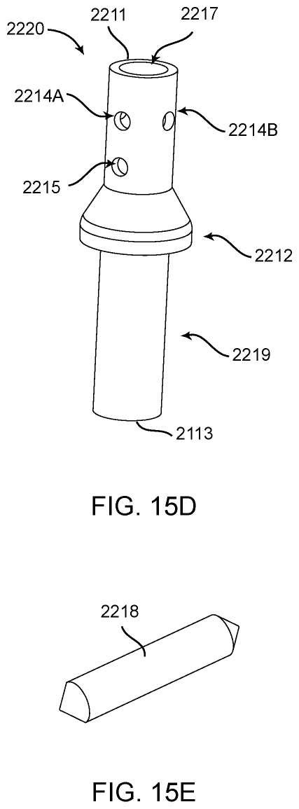

When the blade is in the retracted position as shown in FIG. 3A, the apparatus 102 is suitable for generating plasma. In the retracted position, RF energy is conducted to a tip 146 of the blade 118 from an electrosurgical generator (not shown) via the flow tube 122. An inert gas, such as helium, is then supplied through the flow tube 122 from either the electrosurgical generator or an external gas source. As the inert gas flows over the sharp point 146 of the blade 118 held at high voltage and high frequency, a cold plasma beam is generated. While other inert gases are known and are used in generating plasma for surgical applications, e.g. argon, helium is preferred due to its simple molecular structure which translates into the following advantages: (i) Helium can be ionized with low input of energy; (ii) With only two electrons compared to eighteen for argon, the ionization of helium is more controlled, which produces a more stable and less aggressive plasma beam; and (iii) Helium has high thermal conductivity (10 times higher than argon). In a cold plasma, less than 0.1% of the gas is ionized. Therefore, in a cold helium plasma, more than 99.9% of the highly thermally conductive un-ionized helium is available as a heat sink to remove heat from the application site. These three advantages of helium allow for precise, immediate heating and contraction of the target tissue followed by immediate cooling with minimal depth of thermal effect. Referring to FIG. 15, the depth and width of thermal damage to tissue is illustrated for various devices, for example, a helium-based cold plasma (e.g., Renuvion) device, a CO₂ laser device, an ABC (Argon Beam Coagulation) device, a harmonic device, a bipolar electrosurgical device and a monopolar electrosurgical device. As shown in FIG. 15, among the compared devices, a helium-based cold plasma device in accordance with the present disclosure results in minimal depth and width of thermal damage. The cold plasma generated with helium is ideal for the applications of subdermal skin tightening, coagulation, sculpting and contouring as contemplated herein.

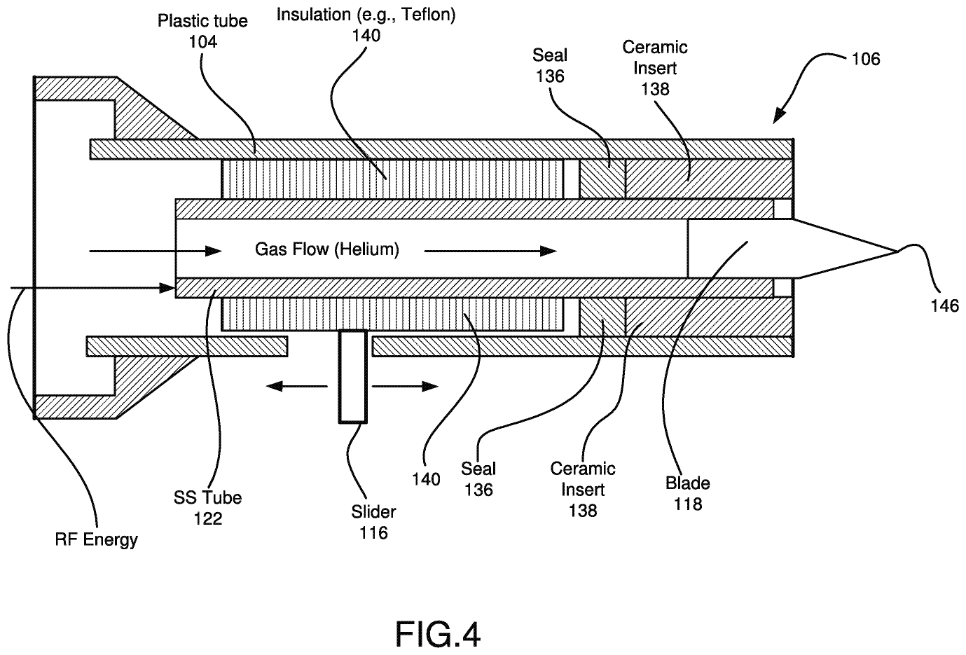

Referring to FIG. 4, the blade 118 is advanced, via slider 116, so the tip 146 is extended past the distal end 106 of the outer tube 104. In this state, the blade 118 can be used for two cutting modes: mechanical cutting and electrosurgical cutting. In the mechanical cutting mode, RF or electrosurgical energy is not applied to the flow tube 122 or blade 118, and therefore, the blade 118 is in a de-energized state. In this mode, the blade 118 can be used to excise tissue via mechanical cutting, i.e., using the blade to make contact with tissue to cut similar to use of a scalpel. After the tissue is removed, the blade 118 may be retracted via the slider 116 and electrosurgical energy and gas may be applied via button 114 to generate a cold plasma beam for cauterization, sterilization and/or hemostasis of the operative patient site.

In the electrosurgical cutting mode, the blade 118 is advanced and used while both electrically energized and enveloped with inert gas flow. This configuration resembles an electrosurgical knife approach, where the electrosurgical energy does the cutting. However, with the addition of the inert gas flow, cuts made show virtually no eschar, with very little collateral damage along the side walls of the cut. The cutting speed is considerably faster, with less mechanical cutting resistance as compared to when the knife blade is not electrically energized, i.e., the mechanical cutting mode. Hemostasis is also affected during this process.

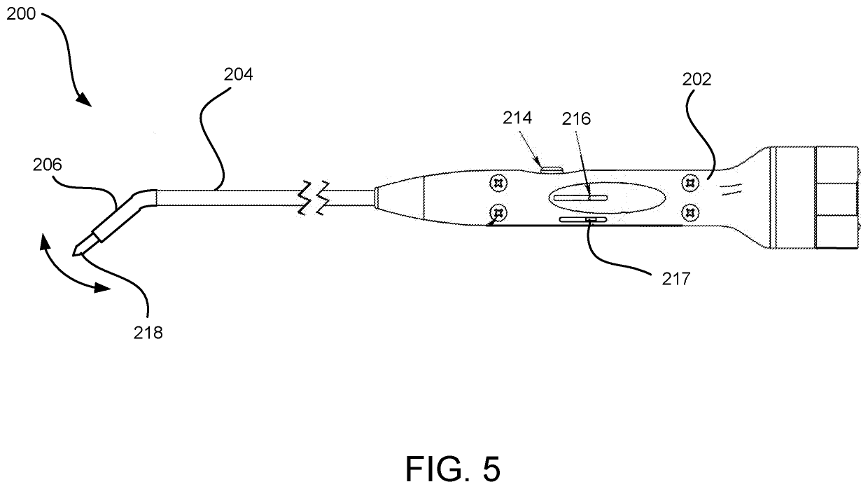

In a further embodiment, the electrosurgical apparatus of the present disclosure will have an articulating distal end. Referring to FIG. 5, the electrosurgical handpiece 200 will have similar aspects to the embodiments described above. In this embodiment, however, the distal end 206, e.g., approximately 2 inches, is flexible to allow it to maneuver at the surgical site. An additional control 217, e.g., a slider, trigger, or the like, is provided in the proximal housing 202 to control the bending of the distal end 206. As in the above described embodiments, a button 214 is provided to apply electrosurgical energy to the blade 218 and, in certain embodiments, enable gas flow through the flow tube. Furthermore, slider 216 will expose the blade 218 at the open distal end 206 upon activation.

In one embodiment, the articulating control 217 will include two wires, one pulling to articulate and one pulling to straighten the distal end 206. The outer tube 204 will be the similar to the design shown in FIG. 2 and will be rigid, preferably made of Ultem™, Lestran™, or similar material, up to the last 2 inches which would be made of a material similar to that of a gastrointestinal (GI) flexible scope. In certain embodiments, a mesh infused TeflonTM or similar material and a flexible insulating material may be positioned inside the outer tube 204 and would allow the distal end 206 to bend at least 45° and not collapse the inner tube carrying the gas. The blade 218 will be made of a flexible metallic material such as Nitinol™ that would be able to bend but would retain its shape in the straightened position. Alternatively, a straight metal blade 218 would be provided with the distal 2 inches made of a linked metal, e.g., stainless steel, tungsten, etc., such that it would still carry a current but would be bendable and the cutting portion of the blade 218 would be attached to the distal end of the linked portion.

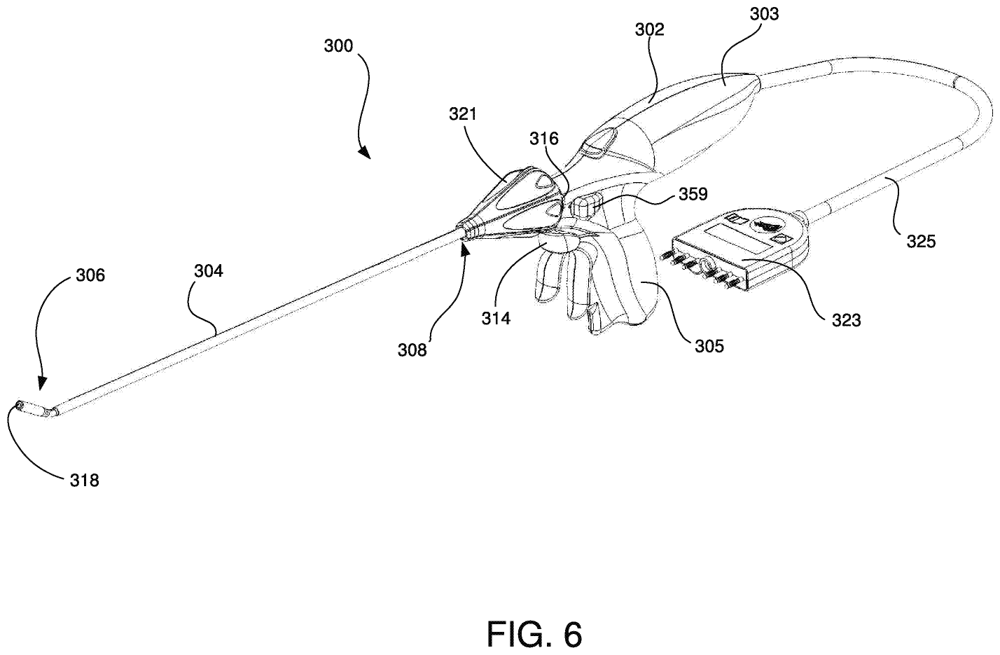

In another embodiment, an electrosurgical apparatus of the present disclosure includes a bent tip applicator or handpiece. Referring to FIG. 6, the handpiece or plasma generator 300 may be configured as a trigger-type handpiece or cold plasma bent tip applicator and will have similar aspects to the embodiments described above. In this embodiment, however, the distal end 306 is pre-bent, e.g., in certain embodiments approximately 28.72 mm, and rotatable to maneuver the distal end 306 at the surgical site 18. The handpiece 300 includes a housing 302 with a handle 305 to facilitate maneuvering of the apparatus by an operator. The handpiece 300 further includes a transformer (not shown) disposed in a proximal end 303 of the housing 302, an activation button 314 for activating the applicator or handpiece to generate plasma configured as a trigger-type button, an insulating tube 304 with a discharge electrode or blade 318 disposed therein. It is to be appreciated that in certain embodiments, the transformer is not disposed in the housing 302, but provided in an appropriate electrosurgical generator. The discharge electrode or blade 318 is coupled to a conductive metal tube (disposed within the insulating tube 304) which is further coupled to a slider button 316, collectively referred to as a slider assembly 319. The slider button 316 moves the metal tube 322 which extends or retracts the discharge electrode or blade 318 beyond the distal end 306 of the insulating tube 304. In one embodiment, the slider button 316 is moved in the distal direction to extend the electrode 318, and the electrode 318 may be retracted by actuating a spring-loaded release button 359. A knob 321 is provided at the proximal end 308 of the insulating tube 304 to enable 360-degree rotation of the insulating tube 304 and thus the distal end 306 of the applicator. It is to be appreciated that the distal end 306 rotates at a predetermined angle relative to the longitudinal axis of the insulating tube 304. Additionally, a connector 323 is provided for coupling the applicator to an electrosurgical generator. In certain embodiments, the connector 323 receives electrosurgical energy and gas which it provides to the applicator or apparatus 300 via cable 325.

As described above, the system of the present disclosure includes an electrosurgical generator unit (ESU), a handpiece (e.g., handpiece 14, 100, 200, 300), and a supply of helium gas. Radiofrequency (RF) energy is delivered to the handpiece by the ESU and used to energize an electrode. When helium gas is past over the energized electrode, a helium plasma is generated which allows for conduction of the RF energy from the electrode to the patient in the form of a precise helium plasma beam. The energy delivered to the patient via the helium plasma beam is very precise and cooler in temperature in comparison to other surgical energy modalities such as laser and standard RF monopolar energy. In one embodiment, Helium is used because it can be converted to a plasma with very little energy. The result is an energy that is unique in its ability to provide tissue heating and cooling almost simultaneously. With the devices and systems of the present disclosure, less than 0.1% of the Helium gas employed is converted to plasma, so >99.9% of the Helium remains in a gaseous state. Helium is eight times more thermally-conductive than air, so the unconverted, or un-ionized, Helium flows across the tissue to draw away excess heat, minimizing any unintended thermal effect.

The unique heating of the devices and systems of the present disclosure makes it a useful surgical tool for the coagulation of subcutaneous soft tissue similar to the LAL and RFAL devices discussed above. As the tip of the handpiece or plasma generator is drawn through the subdermal plane, heating of the tissue results in instant coagulation and contraction of the tissue followed by immediate cooling.

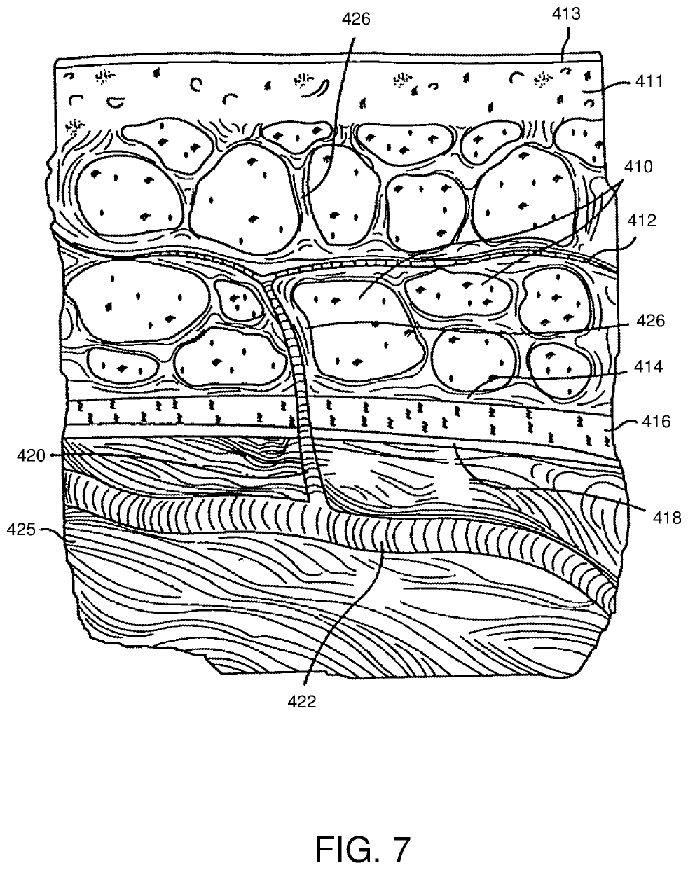

Turning now to FIG. 7, a cross-sectional view of the anatomy of the human cutaneous tissue is illustrated. An epidermis layer 413 overlies the dermis layer 411. Underneath the dermis 411 is a layer of subcutaneous fat 410. Superficial vessels 412 within the fat layer 410 are connected to perforating vessels 420 which in turn are connected to deep vessels 422. Vertical cutaneous ligaments 426 joining tissue layers, are also shown within the fat layer 410. Muscle 425 is covered by a thin layer of deep fascia 418. The fat layer 410 is sheathed by a thin layer of superficial fascia 414. A naturally occurring tissue plane or fascial cleft 416 occurs between the superficial fascia 414 and deep fascia 418.

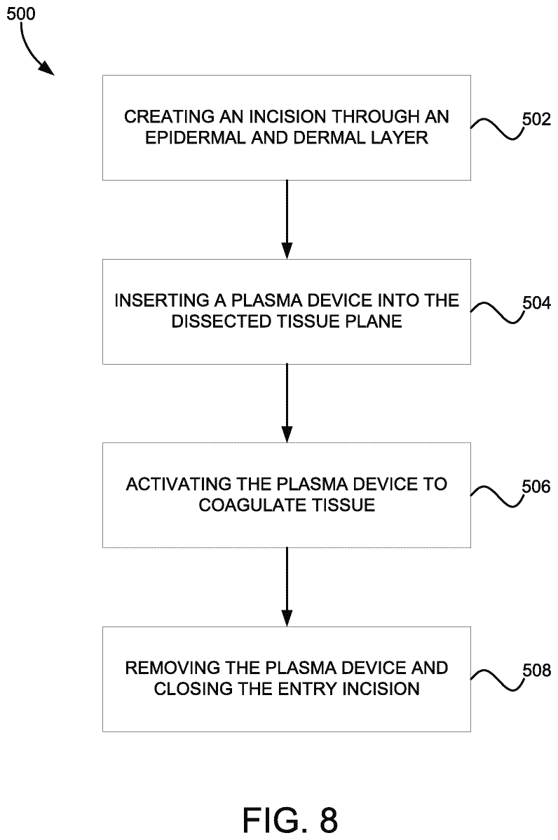

A method of coagulating a subcutaneous layer of tissue will now be described in relation to FIG. 7 and FIG. 8. It is to be appreciated that method may be employed with any of the handpieces or plasma generators described above, for example, plasma generators 14, 100, 200, 300. It is to be appreciated that liposuction may be performed before on the tissue before the method of FIG. 8 is performed.

Initially, in step 502, an incision, i.e., an entry incision, is created through the epidermal 413 and dermal 411 layers of a patient at a location appropriate for a particular procedure.

In step 504, the tip of the plasma generator is inserted into the dissected tissue plane. Next, in step 506, the plasma generator 100, 200, 300 is activated to coagulate and/or ablate tissue to create a desired effect, e.g., (i) tighten tissue (ii) shrink tissue and/or (iii) contour or sculpt the body. After the desired effects are achieved, the plasma generator is removed and the entry incision is closed, in step 508.

A wanding motion may be used with the plasma device, moving the tip back and forth and laterally in order to optimize distribution of the helium gas, plasma and energy to achieve the desired tissue tightening, coagulation, shrinking or sculpting.

Custom tips for the plasma generators of the present disclosure are contemplated to optimize gas and energy distribution. See, for example, commonly-owned U.S. patent application Ser. No. 15/717,643 filed Sep. 27, 2017 entitled “DEVICES, SYSTEMS AND METHODS FOR ENHANCING PHYSIOLOGICAL EFFECTIVENESS OF MEDICAL COLD PLASMA DISCHARGES” and commonly-owned PCT Patent Application No. PCT/US2016/064537 filed Dec. 2, 2016 entitled “DEVICES, SYSTEMS AND METHODS FOR IMPROVED MIXING OF COLD PLASMA BEAM JETS WITH AMBIENT ATMOSPHERE FOR ENHANCED PRODUCTION OF RADICAL SPECIES”, the entire contents of both of which is hereby incorporated by reference.

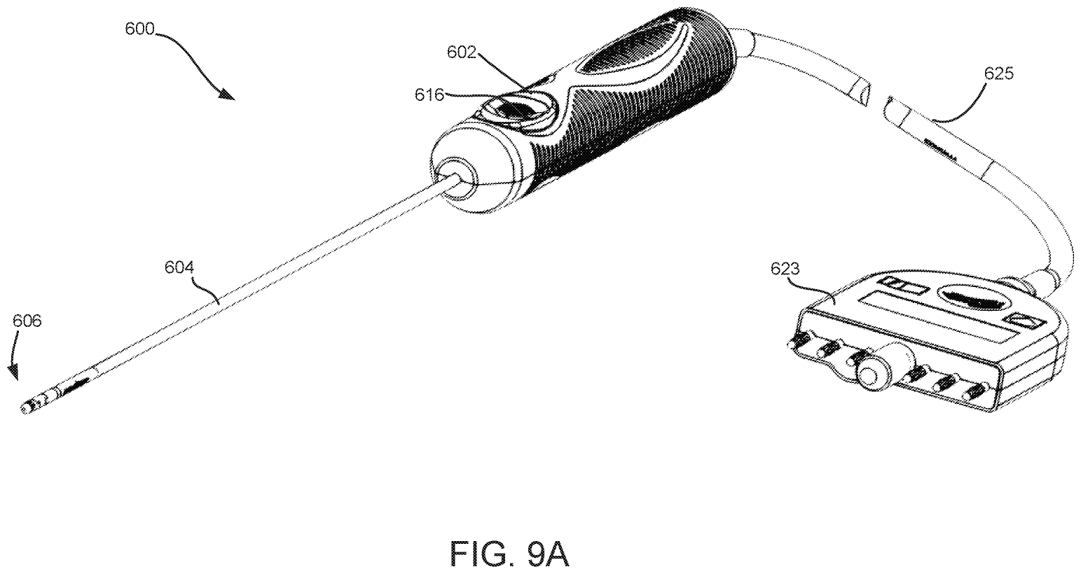

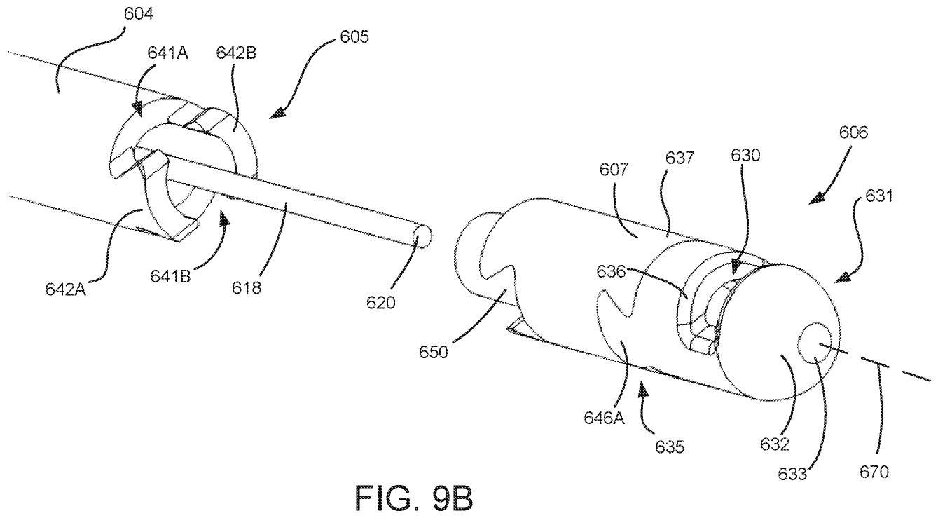

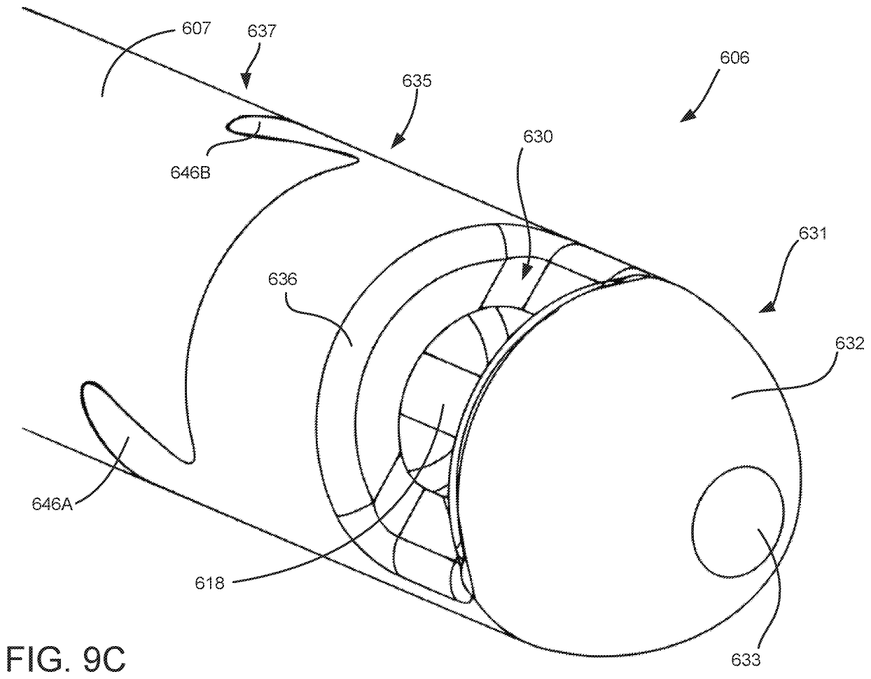

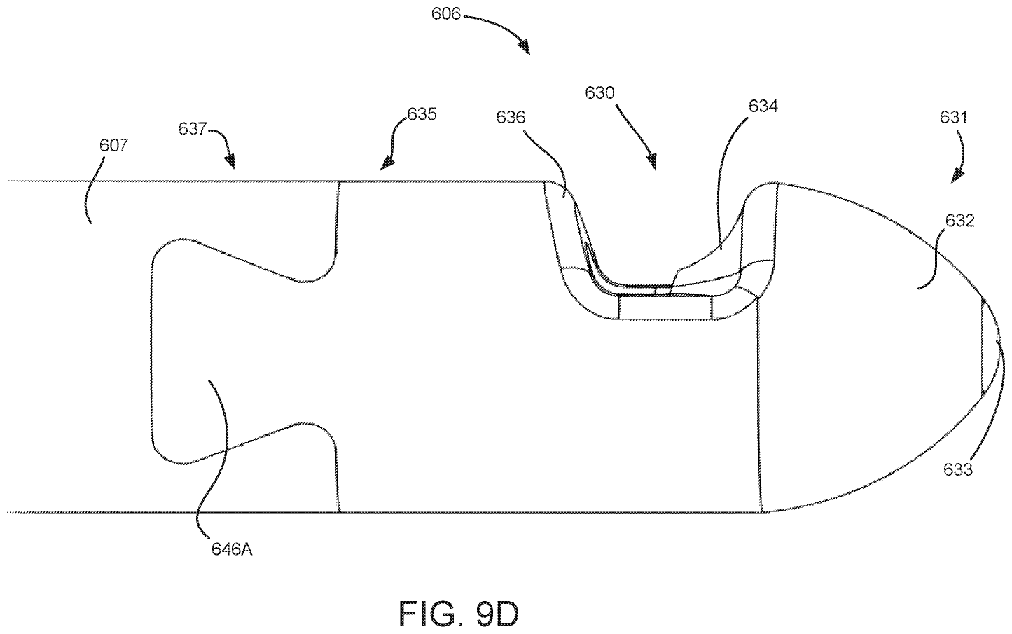

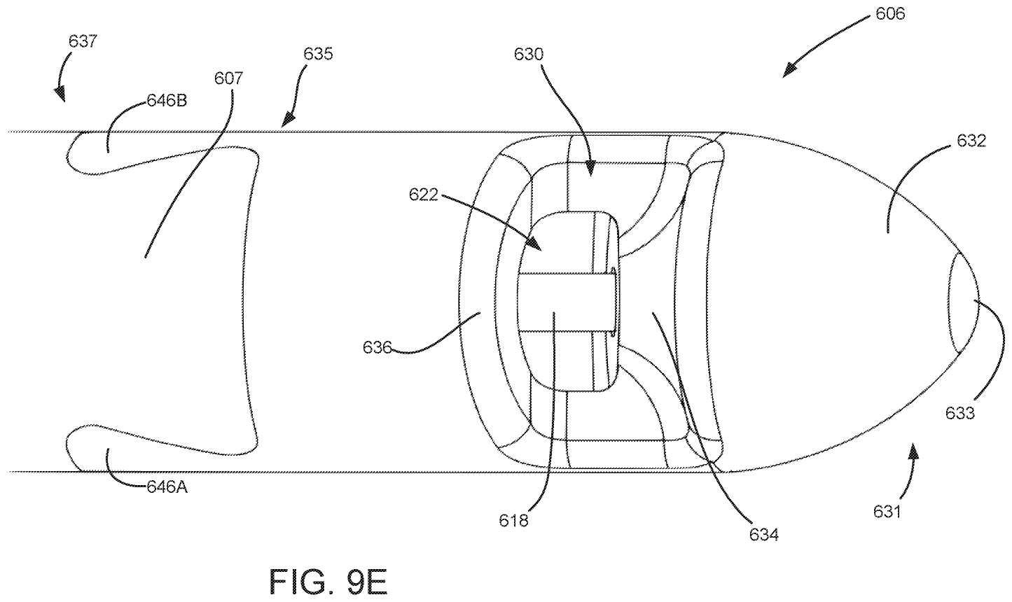

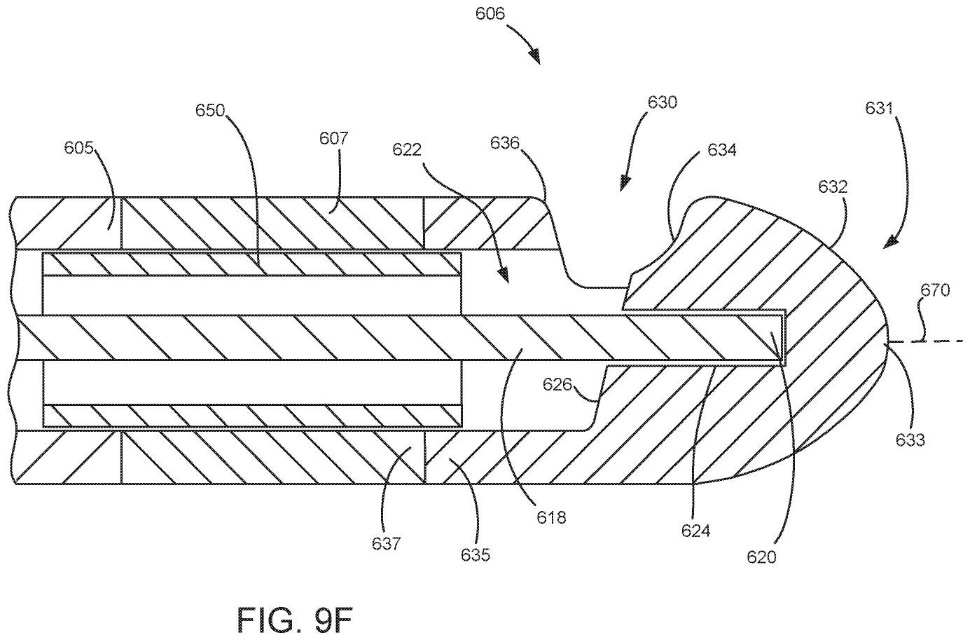

For example, referring to FIG. 9A, a plasma device or electrosurgical apparatus 600 is shown in accordance with an embodiment of the present disclosure. It is to be appreciated that apparatus 600 may be employed to perform method 500 described above.

As shown in FIG. 9A, apparatus 600 includes a housing or handle 602, a gas conduit or shaft 604, a distal tip 606, a cable 625, and a connector 623. Connector 623 is provided for coupling the apparatus 600 to an electrosurgical generator. The connector 623 receives electrosurgical energy and inert gas from the electrosurgical generator and/or a gas source, which the connector 623 provides to the apparatus 600 via cable 625. Apparatus 600 may include one or more selectable user controls (e.g., buttons, sliders, etc.) 616. The user selectable control 616 may be pressed or actuated by a user to activate apparatus 600. Activation of apparatus 600 causes the electrosurgical generator coupled to apparatus 600 to provide electrosurgical energy and/or gas to apparatus 600.

Apparatus 600 includes an electrically conducting member or electrode 618 (shown in FIG. 9B), e.g., a conductive rod, wire, or other suitable electrode, disposed through shaft 604. In one embodiment, electrode 618 is made of tungsten, however, other suitable materials are contemplated to be within the scope of the present disclosure. Shaft 604 is made of a non-conducting material and is configured to provide inert gas to tip 606. Electrode 618 is configured to provide electrosurgical energy to tip 606. In some embodiments, shaft 604 is configured to enable a degree of flexibility (e.g., bending of shaft 604) to facilitate the insertion of tip 606 and shaft 604 through subdermal tissue during electrosurgical procedures performed with apparatus 600.

Referring to FIGS. 9B-9F, various views of distal tip 606 are shown in accordance with the present disclosure.

Apparatus 600 further includes a tubular insert or support tube 650 (e.g., a thin-walled stainless steel tube) and injection-molded coupling member 607. Shaft 604, tube 650, coupling member 607, and tip 606 are disposed along a longitudinal axis 670. In one embodiment, the distal end 605 of shaft 604 includes male interlocking members or tabs 642A, 642B and female interlocking slots 641A, 641B, which are each disposed between male interlocking members 642A, 642B. Tip 606 includes a distal end 631 and a proximal end 635. The proximal end 635 of tip 606 includes male interlocking members or tabs 646A, 646B and female interlocking slots, which are each disposed between male interlocking members 642A, 642B. Tip 606 includes a port 630 disposed through a side wall of tip 606 and oriented in a radial direction traverse to axis 670. Tip 606 further includes interior 622, which includes an inner wall 626 having a slot or channel 624. Inner wall 626 is angled or slanted such that wall 626 transverses the longitudinal axis 670 at a predetermined angle.

In one embodiment, to couple tip 606 to shaft 604, a proximal portion of tube 650 is disposed in and glued to the interior of shaft 604 and a distal portion of tube 650 is disposed in and glued to the interior 622 of tip 606. Thereafter, coupling member 607 is created by injection molding a suitable non-conducting material (e.g., thermoplastic) over tube 650 and between the distal end 605 of shaft 604 and the proximal end 635 of tip 606. When the injection moldable material is applied, the injection moldable material fills the space between end 605 of shaft 604 and end 635 of tip 606 and enters the female interlocking slots disposed between male interlocking members 642, 646. Thereafter, the injection moldable material solidifies and coupling member 607 is formed. In the solidified state, coupling member 607 interacts with interlocking features 642, 641, 646, of shaft 604 and tip 606 to secure shaft 604 to tip 606.

When tip 606 is coupled to shaft 604, electrode 618 extends from the interior of shaft 604 through tube 650 and interior 622. A distal end 620 of electrode 618 is securely received by the slot 624 of interior 622 such that a distal portion electrode 618 is disposed adjacent to port 630. Port 630 is disposed through a side wall of tip 606 such that port 630 is oriented in a radial direction with respect to axis 670. Port 630 includes a curved surface 634 having a concavely rounded edge perimeter 636 disposed adjacent to the exterior walls of tip 606. Distal end 631 of tip 606 includes an exterior surface or wall 632 shaped as an elliptic paraboloid or an elliptical cone with a blunted or rounded tip 633 converging toward distal end 631.

It is to be appreciated that tip 633, wall 632, and edge 636 are shaped such that when tip 606 is moved through subcutaneous tissue, the curved surfaces 633, 632, 636 of tip 606 enable tip 606 to glide through the subcutaneous tissue with minimal resistance.

When inert gas, such as Helium, is provided through shaft 604 and into interior 622 and electrode 618 is energized, at least some of the inert gas is ionized and plasma is generated within interior 622 of tip 606. The angled wall 626 of interior 622 is configured to guide the plasma generated and the remaining inert gas (i.e., the gas passing over electrode 618 that is not ionized) toward the exterior of tip 606 via port 630 both radially and distally at the same time. Port 630 arcs about axis 670 at a predetermined arc length. In one embodiment, port 630 arcs about axis 670 such that the arc length of port 630 is slightly less than half the circumference of tip 606. In this way, the plasma generated by tip 606 may exit port 630 and be used to provide an 180° tissue treatment area about the longitudinal axis 670. It is to be appreciated that the arc length of port 630 shown is merely exemplary and that other arc lengths are contemplated to be within the scope of the present disclosure.

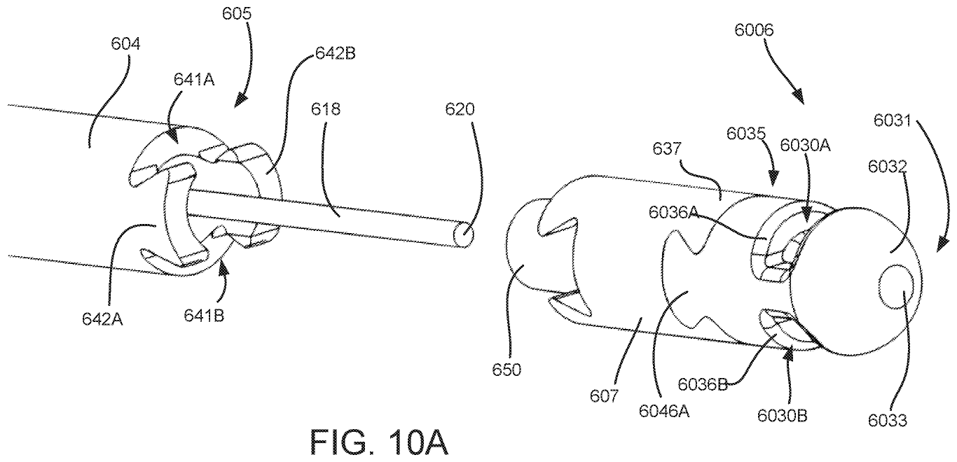

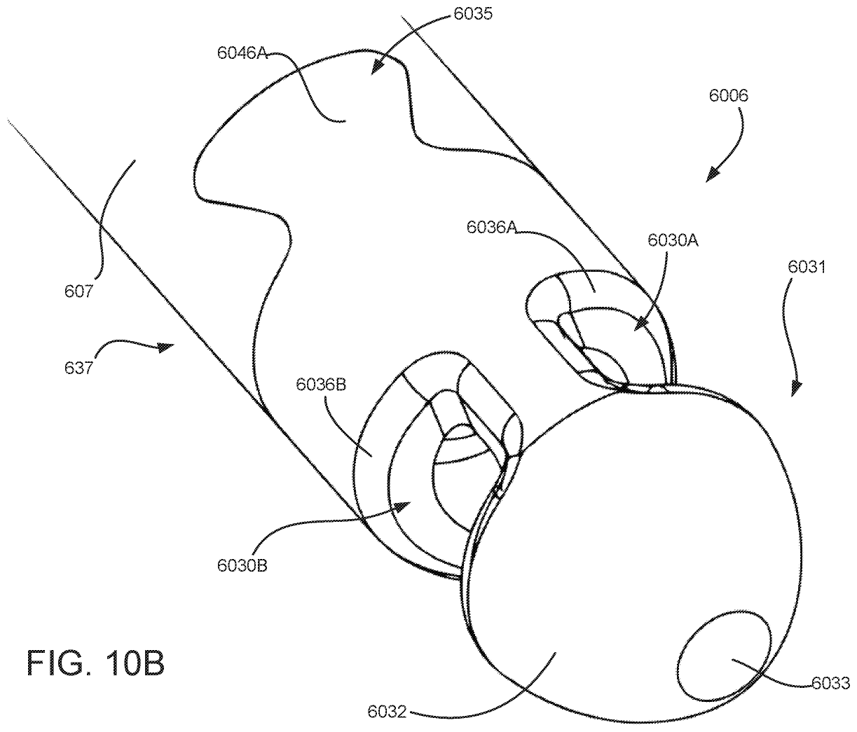

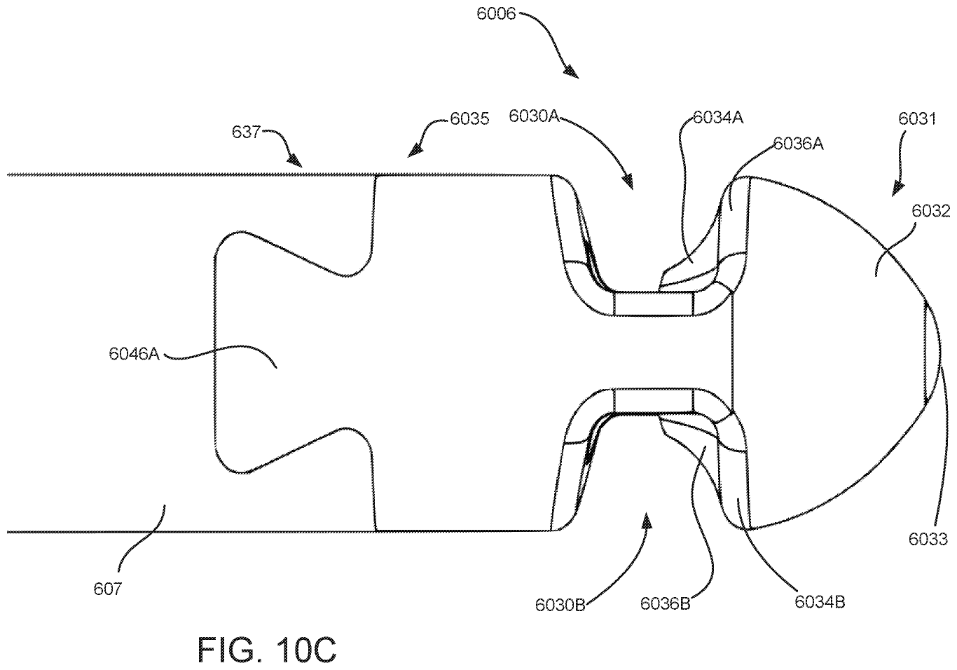

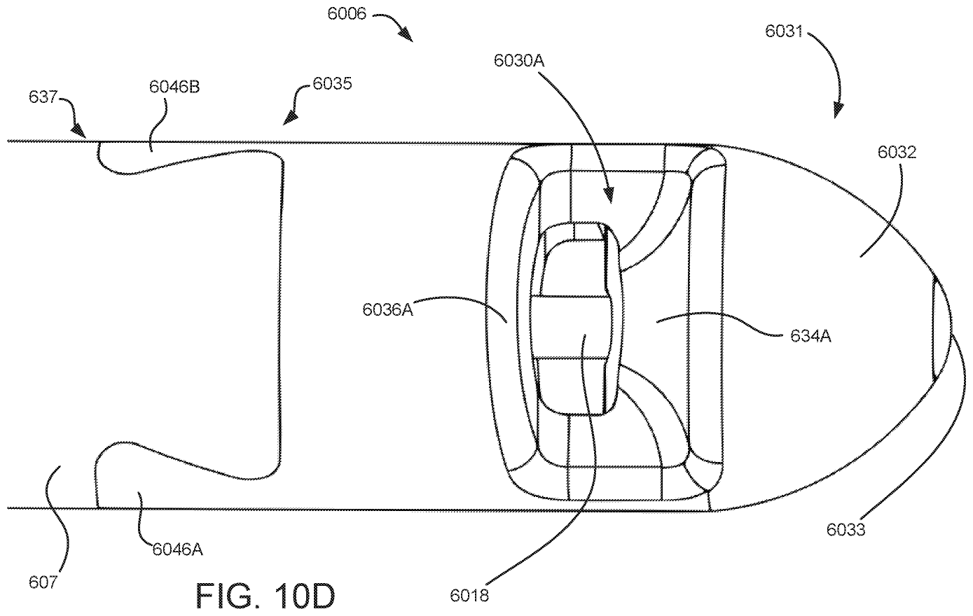

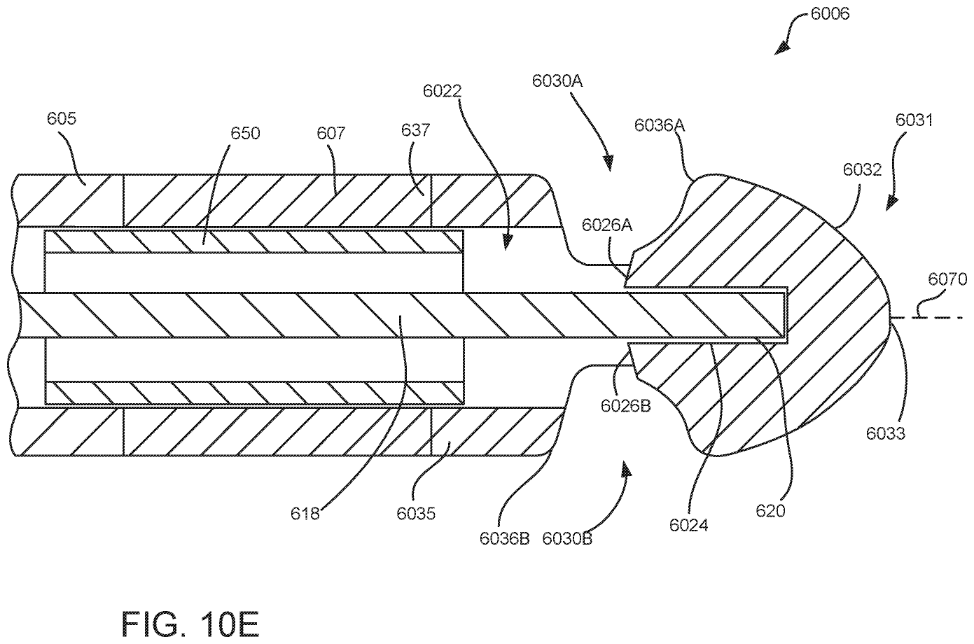

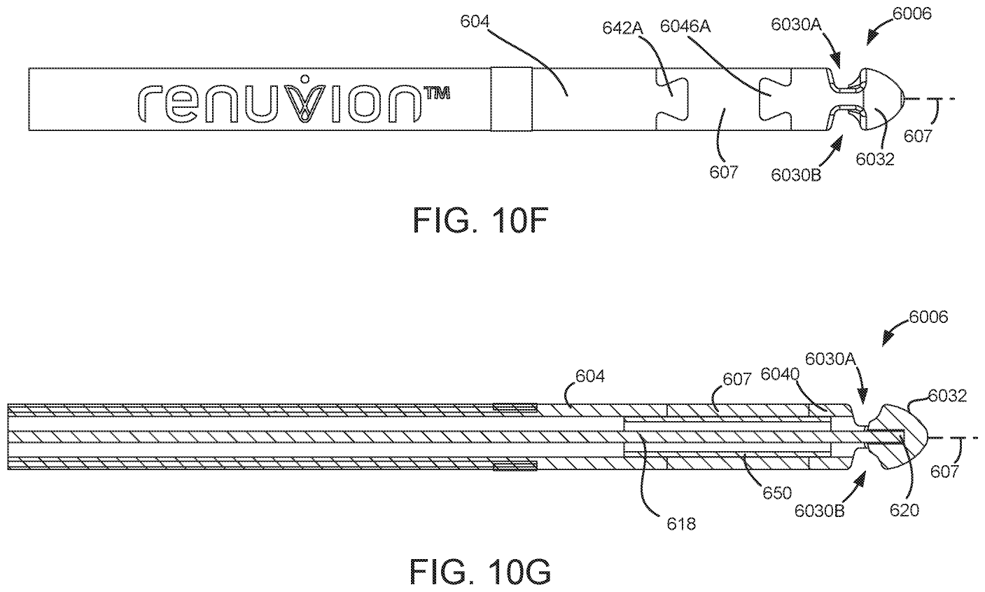

Although tip 606, as shown in FIGS. 9B-9F, includes a single port 630 capable of providing 180° treatment of tissue, in another embodiment, tip 606 may be configured with at least a second port for providing a 360° treatment area for tissue about the longitudinal axis 670. For example, referring to FIGS. 10A-10G, a tip 6006 including first and second ports 6030A, 6030B and coupled to shaft 604 for use with apparatus 600 is shown in accordance with another embodiment of the present disclosure. It is to be appreciated that tip 6006 is configured with the same features as tip 606, described above, except for the additional features provided below. Reference numerals in FIGS. 10A-10G numbered similarly to numerals in FIGS. 9B-9F denote an element or component configured in the same manner (e.g., 632 and 6032 denote elements configured with the same features).

Ports 6030A, 6030B are each configured with the features of port 630 described above. Ports 6030A, 6030B are diametrically opposed with respect to axis 670, such that ports 6030A, 6030B are oriented in opposite directions. As best seen in FIG. 10E, in this embodiment, the interior of tip 6006 includes a wall having a first portion 6026A and a second portion 6026B. The first portion 6026A is angled to direct inert gas and plasma generated to exit via port 6030A and the second portion 6026B is angled to direct inert gas and plasma generated to exit via port 6030B. In this way, tip 6006 may be configured such that gas and plasma exit both ports 6030A, 6030B simultaneously and tissue disposed various positions 360° about axis 670 exterior to tip 6006 may be treated using apparatus 600. It is to be appreciated that tip 6006 shown in FIGS. 10A-10G is secured to shaft 604 using the injection molding process to form coupling member 607 described above.

Although distal tip 606 is shown in FIGS. 9B-9F and described above as being coupled to shaft 604 via injection molded coupling member 607, in other embodiments, tip 606 may be coupled to shaft 604 using other techniques in accordance with the present disclosure. Below, distal tips for use with apparatus 600 or another electrosurgical apparatus are provided including various techniques for assembling each tip and coupling each tip to the shaft of an electrosurgical apparatus, such as apparatus 600.

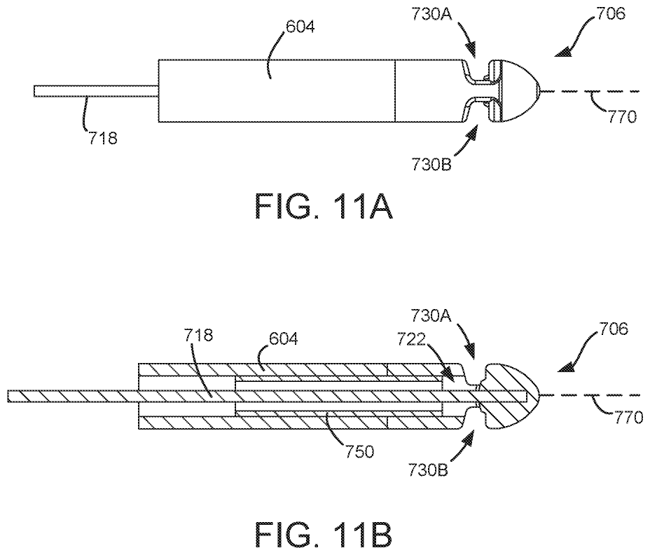

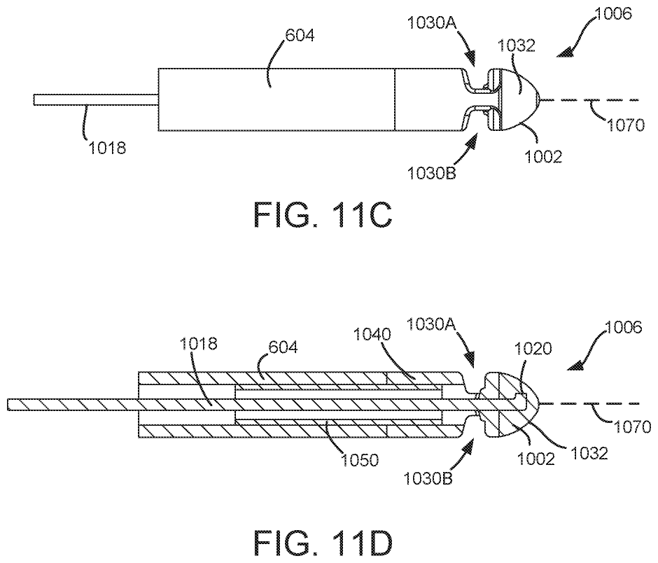

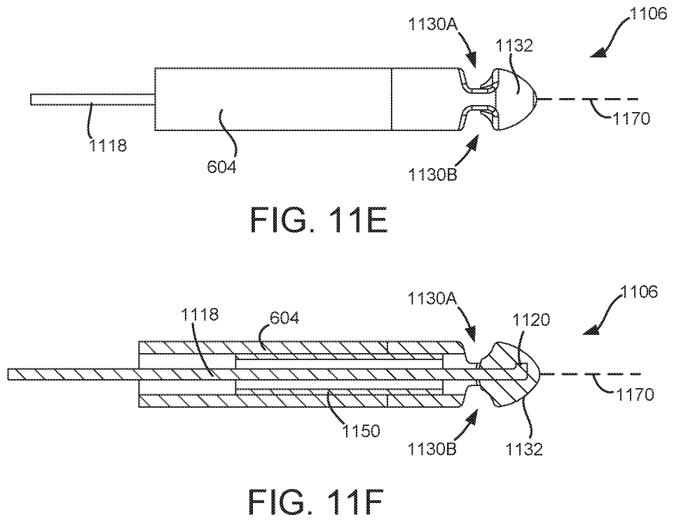

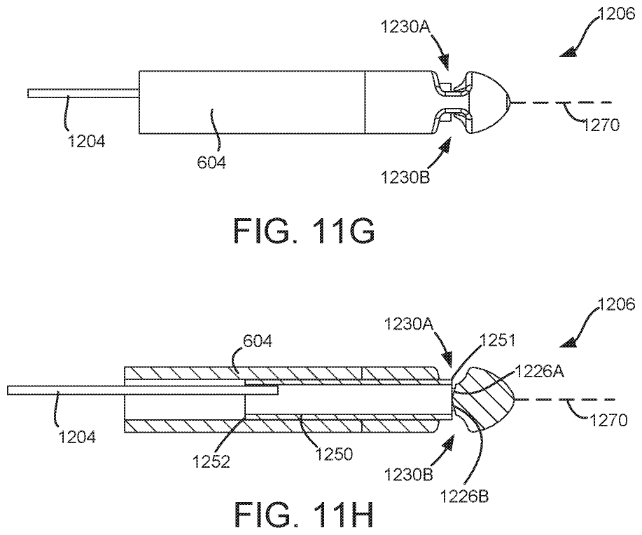

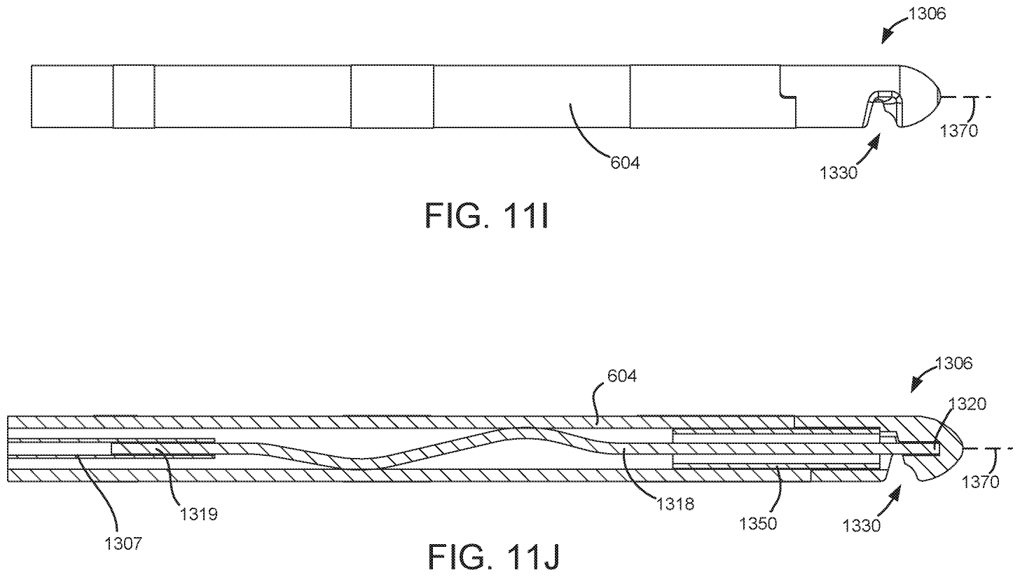

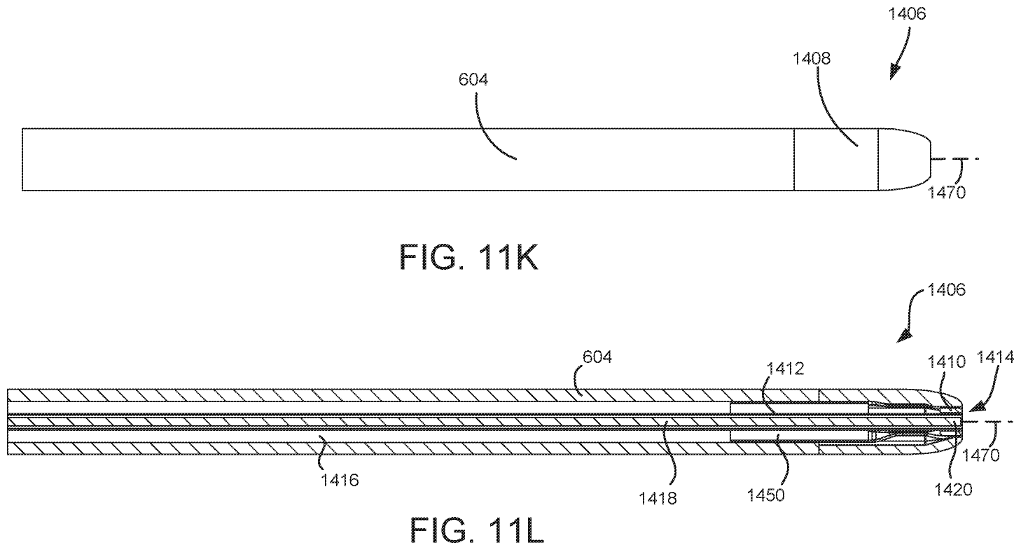

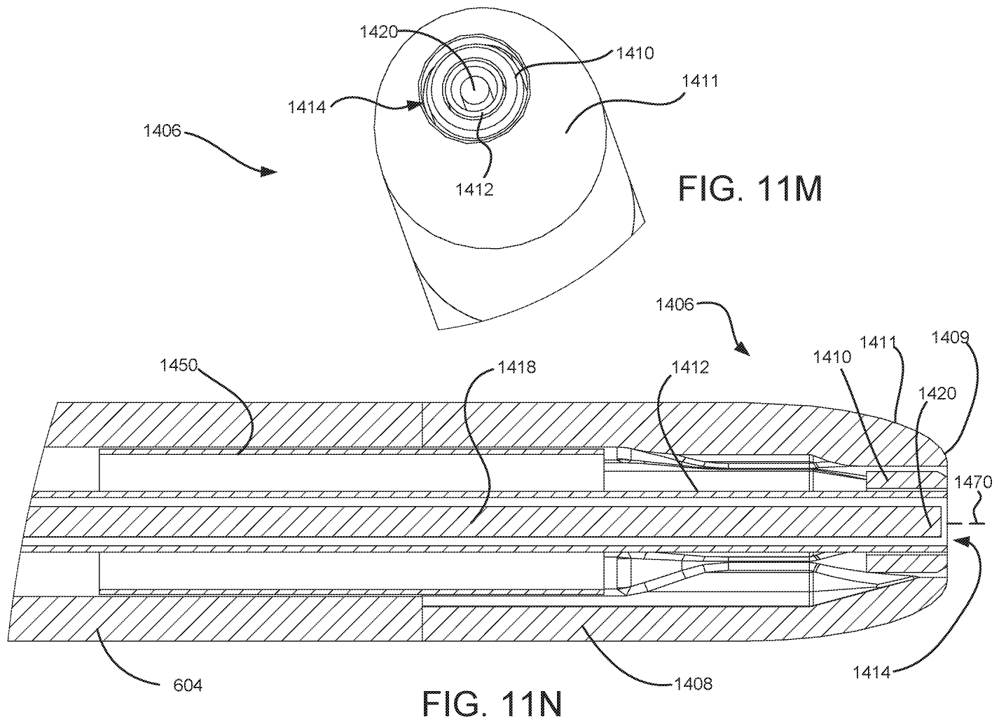

Referring to FIGS. 11A and 11B, a distal tip 706 for use with an electrosurgical apparatus, such as apparatus 600, is shown coupled to shaft 604 and disposed along axis 770 in accordance with another embodiment of the present disclosure. Tip 706 is shaped in a similar manner to tip 606, described above. Tip 706 is disposed adjacent to shaft 704 and a tube 750 is disposed through the distal end of shaft 604 into the interior of shaft 604 and through the proximal end of tip 706 into the interior 722 of tip 706. An adhesive is used to bond the tube 750 to the interior of shaft 604 and the interior 722 of tip 706, thereby coupling tip 706 to shaft 604. Tube 750 provides support to the connection or junction points between shaft 604 and tip 706 to prevent bending at the connection or junction point. It is to be appreciated that tube 750 may be made of conductive or non-conductive materials in various embodiments of the present disclosure.

Referring to FIGS. 11C and 11D, a distal tip 1006 for use with an electrosurgical apparatus, such as apparatus 600, is shown coupled to shaft 604 and disposed along axis 1070 in accordance with another embodiment of the present disclosure. Distal tip 1006 includes a molded cap 1002. Cap 1002 is formed by injection molding a suitable non-conducting moldable material (e.g., thermoplastic) on a distal portion 1040 of tip 1006. Cap 1002 includes a surface 1032, which is configured in the same manner and includes the same features as surface 632, described above. The distal end 1020 of electrode 1018 is disposed in a channel or slot of tip 1006 and cap 1002 is molded over distal end 1020. As shown in FIG. 11 D, while most of electrode 1018 extends along longitudinal axis 1070, the distal tip 1020 is configured to extend perpendicularly to longitudinal axis 1070. In this way, after cap 1002 is molded over distal end 1020, electrode 1018 is prevented from moving along axis 1070. Thus, distal end 1020 in cap 1002 is configured to hold tip 1006 and shaft 604 together, prevent tip 1006 from being removed from shaft 1004, and provide additional rigidity. Tube 1050 is disposed in the interior of shaft 1004 and tip 1006 and provides support to the connection or junction points between shaft 604 and tip 1006 to prevent bending at the connection or junction point. In one embodiment, an adhesive is used to bond tube 1050 to the interior of shaft 604 and tip 1006.

Referring to FIGS. 11E and 11F, a distal tip 1106 for use with an electrosurgical apparatus, such as apparatus 600, is shown coupled to shaft 604 and disposed along axis 1170 in accordance with another embodiment of the present disclosure. In this embodiment, tip 1106 is formed via injection molding a suitable non-conducting moldable material (e.g., ceramic) over the distal portion of tube 1150 and electrode 1118. Tip 1106 is configured in the same manner and includes the same features as tip 606 described above (e.g., the interior of tip 1106 is configured in the same manner as interior 622 described above and ports 1130A, 11308 are configured in the same manner as ports 6030A, 6030B). After tip 1106 is molded over the distal end of insert 1150 and the distal end 1120 of electrode 1118, the perpendicularly extending distal end 1120 of electrode 1118 is configured to prevent electrode 1118 from moving along axis 1170 and hold tip 1106 and shaft 604 together.

Referring to FIGS. 11G and 11H, a distal tip 1206 for use with an electrosurgical apparatus, such as apparatus 600, is shown coupled to shaft 604 and disposed along axis 1270 in accordance with another embodiment of the present disclosure. The distal end 1251 of a tube 1250 extends within and is glued to interior of tip 1206 until end 1251 is disposed adjacent to ports 1230A, 12308 and portions 1226A, 12268 of a wall 626 of the interior of tip 1206. The proximal end 1252 of tube 1250 extends within and is glued to the interior of shaft 604. Tube 1250 is made of a conductive material (e.g., stainless steel) and is configured as an electrode. Furthermore, in this embodiment, a conductive wire 1204 is coupled to tube 1250 and receives electrosurgical energy via a power source (e.g., via cable 626 and connector 623 in the manner described above with respect to electrode 618). In this way, when inert gas is provided through the interior of shaft 604 and the interior of tube 1250 and electrosurgical energy is provided via wire 1204 to tube 1250, plasma is formed in tube 1250 and ejected from distal end 1251 of tube 1250 and ports 1230A, 1230B. In addition to serving as an electrode, tube 1250 provides support to the connection or junction points between shaft 604 and tip 1206 to prevent bending at the connection or junction point.

It is to be appreciated that although each distal tip shown in FIGS. 11A-11H is shown with dual ports, each of the embodiments may also be configured with a single port in accordance with the present disclosure.

Referring to FIGS. 111 and 11J, a distal tip 1306 for use with an electrosurgical apparatus, such as apparatus 600, is shown coupled to shaft 604 and disposed along axis 1370 in accordance with another embodiment of the present disclosure. Tip 1306 includes a single port 1330 and is coupled to shaft 604 using tube 1350. In this embodiment, a proximal end 1319 of electrode 1318 is coupled to a stranded wire 1307 within the interior of shaft 604. A proximal end of stranded wire 1307 is coupled within housing 602 of apparatus 600 (shown in FIG. 9A) to an electrosurgical generator via cable 625 and connector 623. In one embodiment, the proximal end of the stranded wire 1307 is coupled to one or more conductors in cable 625. Stranded wire 1307 is configured to provide electrosurgical energy to distal end 1320 of electrode 1318 such that plasma is formed when electrode 1318 is energized and inert gas is provided via shaft 604 to the interior of tip 1306.

It is to be appreciated that although the embodiment of tip 1306 shown in FIGS. 11I-11J is shown with a single port 1330, the embodiment of tip 1306 shown in FIGS. 11I-11J may also be configured with dual ports 1306 (i.e., disposed in diametrically opposed positions about axis 1370) in accordance with the present disclosure.

Referring to FIGS. 11K, 11L, 11M, 11N, a distal tip 1406 for use with an electrosurgical apparatus, such as apparatus 600, is shown coupled to shaft 604 and disposed along axis 1470 in accordance with another embodiment of the present disclosure. Molded tip 1406 includes a port 1414 that is oriented toward axis 1470 (i.e., a straight-firing port). The proximal portion of tube 1450 is glued to the interior of shaft 604 and tip 1406 is injection molded (e.g., using thermoplastic or another suitable material) over the distal portion of tube 1450. Tip 1406 includes a port 1414. In this embodiment, the electrosurgical apparatus tip 1406 is coupled to (e.g., apparatus 600) includes a tube 1412 disposed around electrode 1418, a plunger cap 1410, and a tubular insulator 1416 lining the inner wall of shaft 604. Tube 1412 is coupled to plunger cap 1410. Plunger cap 1410 is disposed within port 1414 in a distal end 1409 of tip 1406 and around the distal end 1420 of electrode 1418. Cap 1410 is configured to prevent debris from entering the interior of tip 1406 (e.g., the space between the exterior of tube 1412 and the interior of tip 1406) while tip 1406 is being used during procedure.

Tube 1412 is moveable along the longitudinal axis 1470 to retract or extend plunger cap 1410 along the longitudinal axis 1470 to reveal the distal end 1420 of electrode 1418. A proximal end of tube 1412 extends into the interior of housing 602 (shown in FIG. 9A) and is coupled to an actuation mechanism for extending or retracting tube 1412 along longitudinal axis 1470. In one embodiment, the actuation mechanism is a trigger accessible by a user on the housing 602. When a user engages the trigger, the tube 1412 is retracted (i.e., moved proximally) along axis 1470 and when the user disengages the trigger, the tube 1412 is extended (i.e., moved distally) along axis 1470.

In another embodiment, the actuation mechanism may be an electric motor controllable via a button or other selection means to extend or retract tube 1412 along axis 1470. It is to be appreciate that the distal portion of tip 1406 includes a concavely shaped surface 1411 that converges toward end 1409 to enable tip 1406 to travel through subdermal tissue with minimized friction.

In use, initially, plunger cap 1410 is in an extended position and distal end 1420 of electrode 1418 is covered. After tip 1406 is inserted through subdermal tissue to perform an electrosurgical procedure, the actuation mechanism is engaged by the user to retract tube 1412 and plunger cap 1410 along axis 1470 to expose tip 1420 of electrode 1418. Thereafter, inert gas is provided via tube 1412 to tip 1406 and electrosurgical energy is applied to electrode 1418 to generate plasma that is ejected from port 1414 to perform the electrosurgical procedure.

It is to be appreciated that, although distal tips in the embodiments above are shown and described as being fixedly coupled to shaft 604, in some embodiments, the distal tips may be configured to be detachably coupled to distal end 605 of shaft 606 via a coupling mechanism (e.g., such as a screw-on threaded connection between the distal tip and end 605 of shaft 604 configured to seal inert gas within the interior of shaft 604 and the distal tip). In this way, different implementations of distal tips (e.g., any of the various embodiments shown in FIGS. 9B-11N) may be used with apparatus 600 in accordance with the present disclosure. In other embodiments, where the various implementations of the distal tip can be fixedly coupled to distal end 605 of shaft 604, the proximal end of shaft 605 is configured to be detachably coupled to housing 602 via a coupling mechanism (e.g., such as a screw-on threaded connection between housing 602 and end 605 of shaft 604 configured to seal inert gas within the interior of shaft 604 and tip 606).

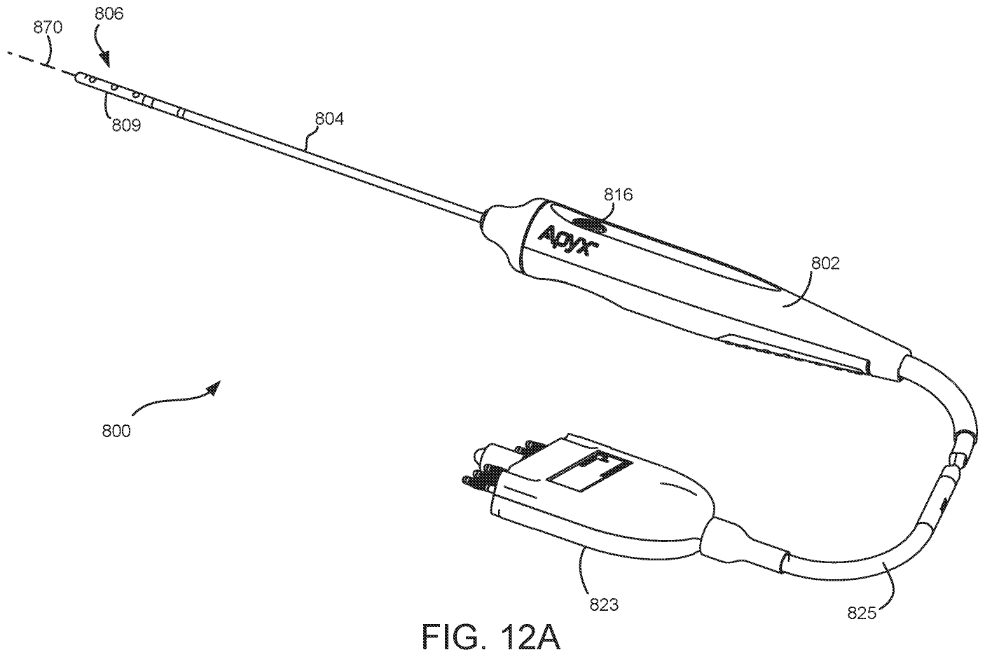

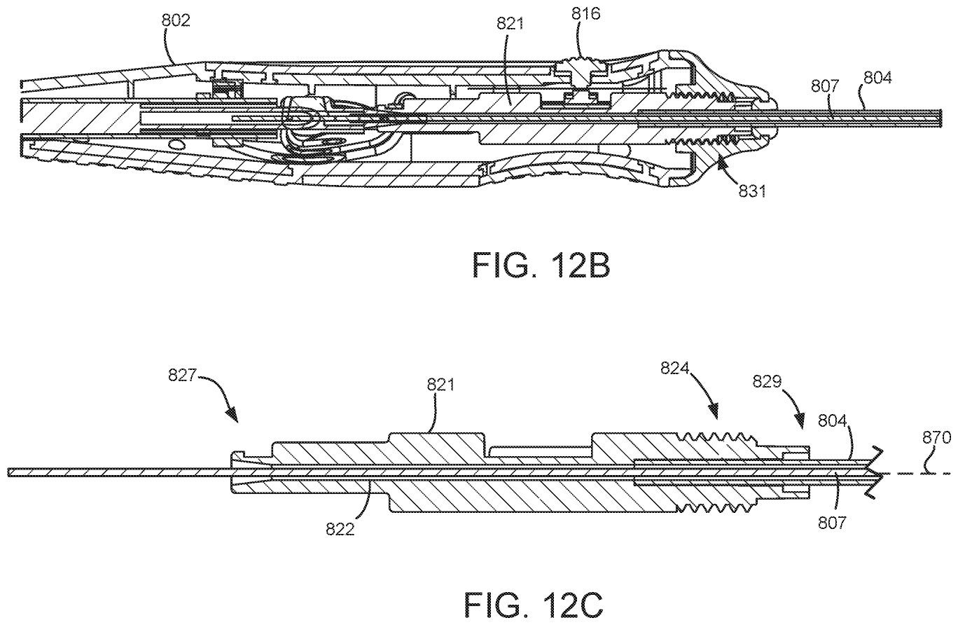

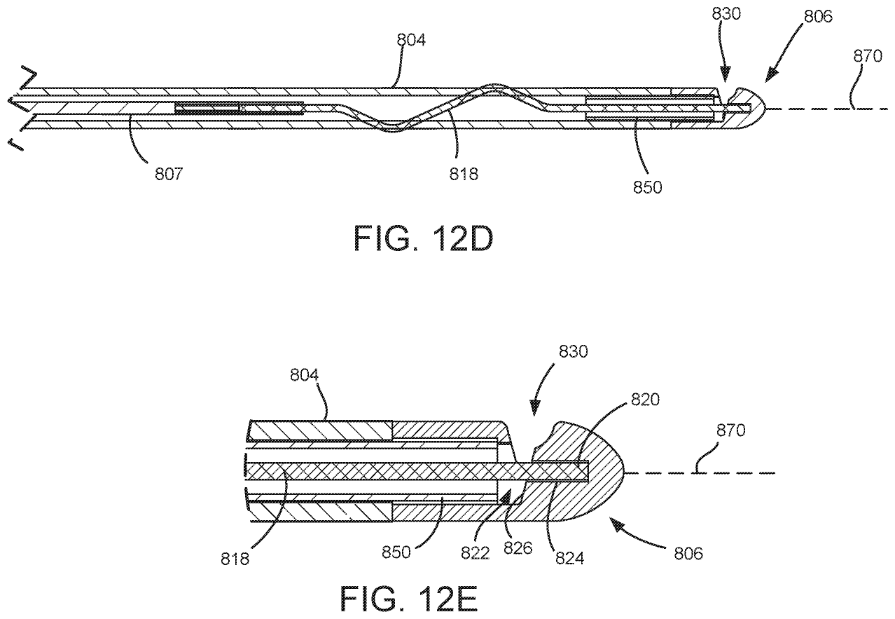

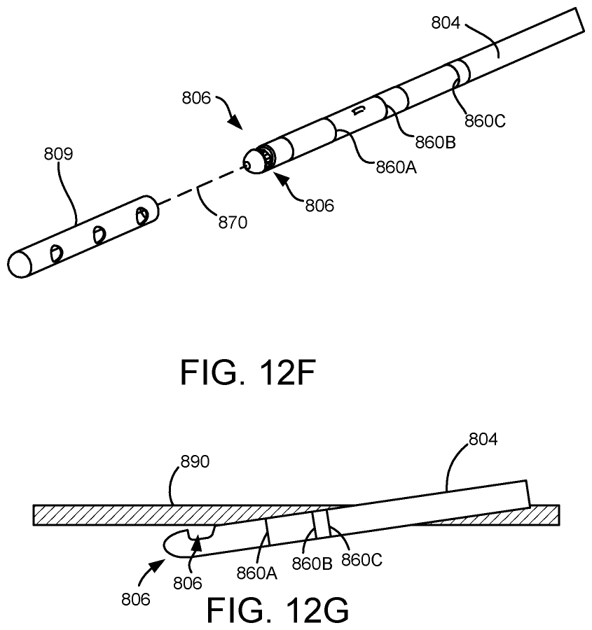

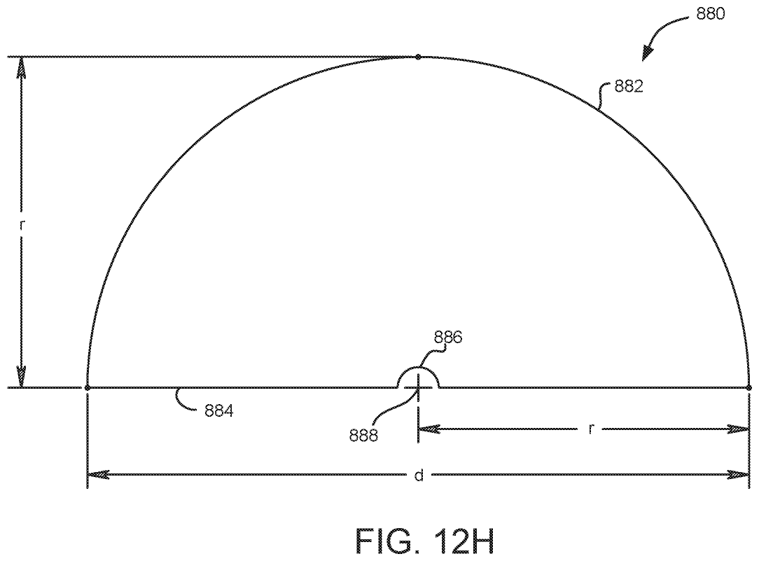

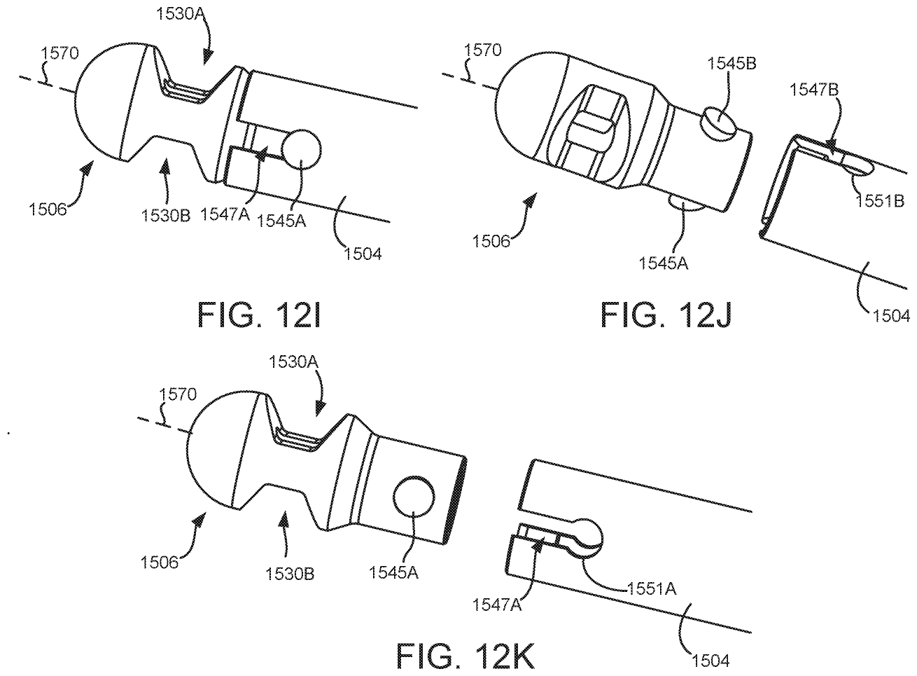

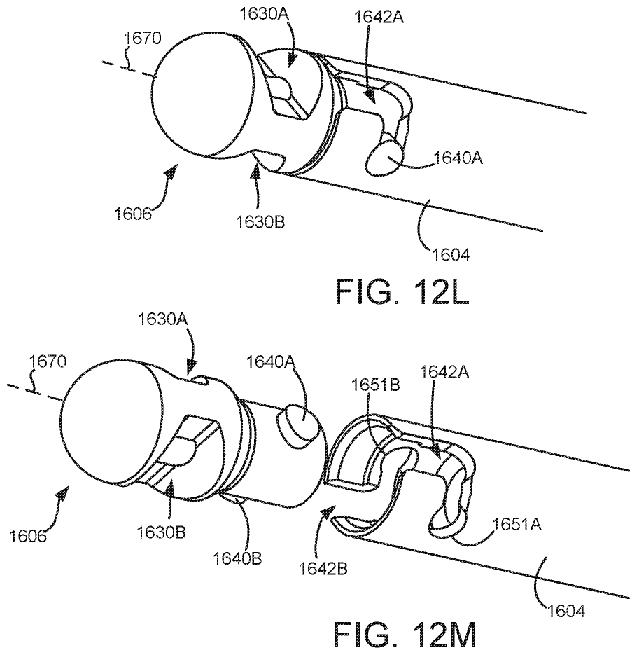

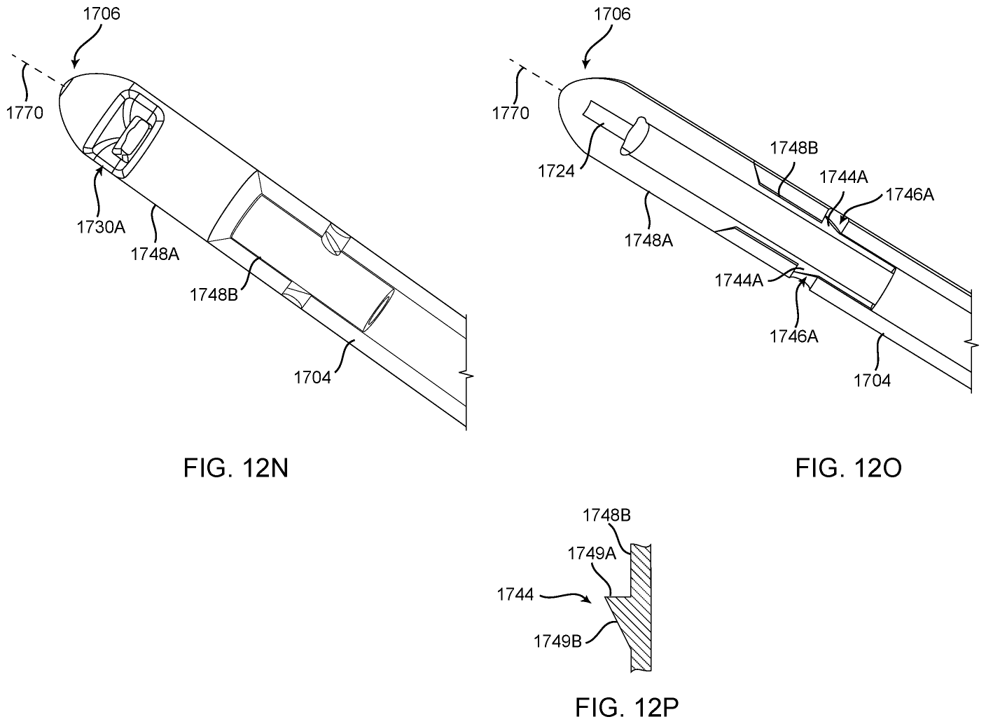

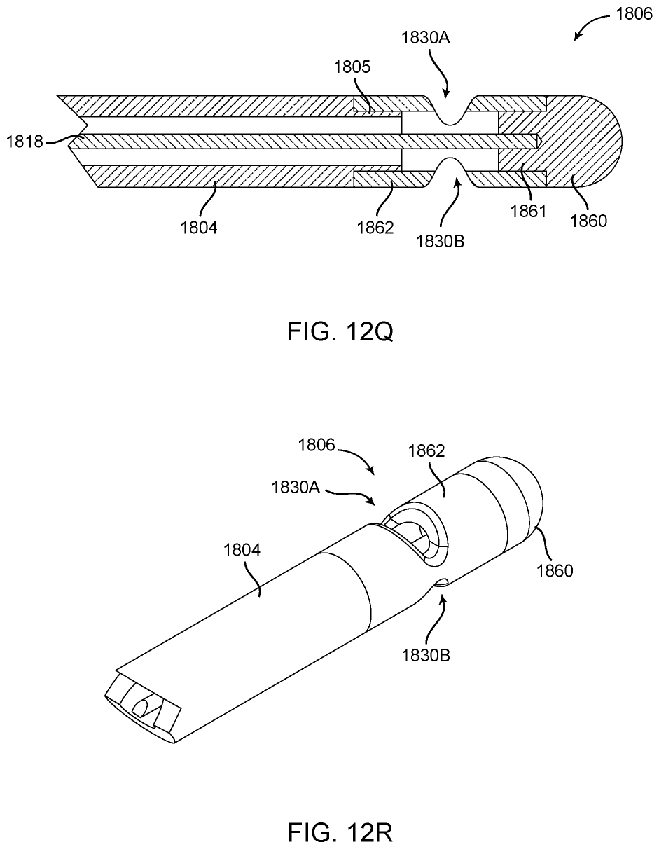

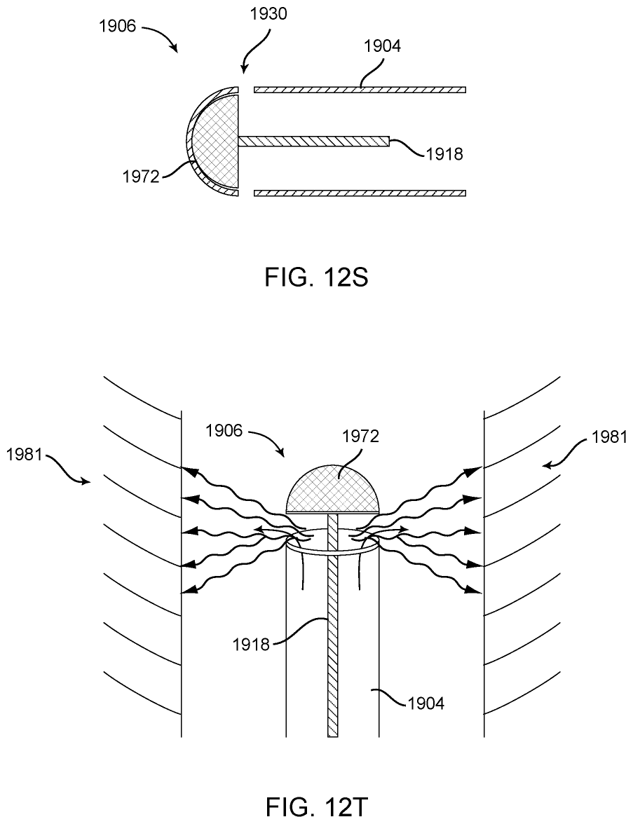

Referring to FIG. 12A, an electrosurgical apparatus 800 is shown in accordance with an embodiment of the present disclosure. Apparatus 800 includes a housing or handle 802, shaft or flow tube 804, distal tip 806, tip protector 809, cable 825, and connector 823. Shaft 804 is coupled to and extends from housing 802 along longitudinal axis 870. Connector 823 is coupled to housing 802 via cable 825. Connector 823 is configured to be coupled to an electrosurgical generator to receive electrosurgical energy and at least one inert gas. The electrosurgical energy and inert gas are provided via cable 825 to housing 802 and via shaft 804 to distal tip 806. Housing 802 includes at least one button 816 for operating apparatus 800 (e.g., causing electrosurgical energy and/or inert gas to be provided to distal tip 806).

Referring to FIG. 12B, a partial cross-sectional view of housing 802, shaft 804, and several internal components of apparatus 800 are shown in accordance with the present disclosure. As shown in FIG. 12B, a proximal portion of shaft 804 is disposed through a distal portion of housing 802 and into a flow tube hub 821. A cross-sectional view of hub 821 is shown in FIG. 12C in accordance with the present disclosure. As shown in FIG. 12C, hub 821 includes a proximal end 827 and a distal end 829. Hub 821 further includes an internal channel 822 extending along axis 870 from proximal end 827 to distal end 829. The distal end 829 of hub 821 is configured to receive a proximal portion of shaft 804. Furthermore, distal end 829 of hub 821 includes a plurality of threads 824 disposed around the exterior of hub 821. Referring to FIG. 12B, threads 824 of hub 821 are received and mated with corresponding threads 831 disposed on interior an wall (or walls) of the distal portion of housing 802 to couple hub 821 to the interior of housing 802.

Referring again to FIG. 12C, via the distal end 829 of hub 821, channel 822 is configured to receive and provide inert gas to the interior of shaft 804 to be further provided to distal tip 806. At least one conducting wire 807 (e.g., in one embodiment at least one stranded wire) is disposed through channel 822 and inserted into the interior of shaft 804. The proximal end of wire 807 is coupled to at least one conductor in cable 825 to receive electrosurgical energy from an electrosurgical generator coupled to connector 823. The distal end of wire 807 extends into the interior of shaft 804 and is coupled to a wire electrode 818, as shown in FIG. 12D, where a partial cross-sectional view of shaft 804 and tip 806 is shown in accordance with the present disclosure. The distal end of wire 807 is coupled to a proximal end of wire electrode 818 to provide electrosurgical energy to electrode 818. Electrode 818 is disposed through the interior of shaft 804 and into the interior of tip 806.

Referring to FIG. 12E, a cross-sectional view of tip 806 and the distal end of shaft 804 is shown in accordance with the present disclosure. The distal end 820 of electrode 818 is disposed in a slot or channel 824 in the interior 822 of tip 806. In one embodiment, tip 806 is made of ceramic material. To couple tip 806 to shaft 804, in one embodiment, the proximal end of insert tube 850 is inserted through the distal end of shaft 804 and is glued (or coupled via other means) to the interior of shaft 804 and the distal end of insert tube 850 is inserted through the proximal end of tip 806 and glued (or coupled via other means) to interior 822 of tip 806. Tube 850 may be made of a conductive or non-conductive material. Tube 850 is configured to provide support to the connection or junction points between shaft 804 and tip 806 to prevent bending at the connection or junction points. It is to be appreciated that tip 806 is configured with similar features to tip 606 described above (e.g., the shape and features of port 830 and the exterior walls of tip 806, etc.) Interior 822 includes an angled or slanted wall 826 that traverses axis 870 at a predetermined angle to directed inert gas provided to interior 822 out of port 830.

It is to be appreciated that although the embodiment of tip 806 shown in FIGS. 12A, 12D, 12E is shown with a single port 830, tip 806 may also be configured with dual ports (in the manner described above with respect to tip 606) in accordance with the present disclosure.

When inert gas is provided to interior 822 of tip 806 and electrosurgical energy is applied to electrode 818, at least some of the inert gas is ionized and plasma is formed in interior 822 and the plasma and remaining inert gas is directed via wall 826 out through port 830 where it is ejected and to be applied to patient tissue.

Referring to FIG. 12F, in one embodiment, apparatus 800 includes a tip protector 809. Tip protector 809 is dimensioned to receive tip 806 via an open end of protector 809, such that tip 806 is disposed in the interior of protector 809 and covered by protector 809. In this way, when apparatus 800 is not in use, protector 809 is configured to protect tip 806 from damage and prevent dirt or other material from entering port 830.

In some embodiments, apparatus 800 may include or employ several safety features. For example, referring to FIGS. 12F, 12G, the distal portion of shaft 804 may include one or more markings 860A, 860B, 860C, each disposed a predetermined distance from the distal end of tip 806 and/or from the center of port 830. The markings 860 are used to aid in a safety practice that instructs users to insert tip 806 through an incision in a tissue surface 890 to a subdermal tissue plane to be treated while apparatus 800 is deactivated. Tip 806 is inserted into the tissue plane up to a predetermined distance and only while tip 806 is pulled in a proximal direction (i.e., drawing tip 806 back toward the incision point) is apparatus 800 activated to apply plasma to the subdermal tissue plane. As tip 806 is pulled proximally toward the incision point, the user is advised (i.e., prior to use of apparatus 800) to deactivate apparatus 800 when port 830 or the distal end of tip 806 is a predetermined distance from the incision point, to prevent applying plasma and treating the tissue of skin surface 890 and the tissue proximate to the incision point, as this would be undesirable. One or more markings 860A-C on shaft 804 may correspond to the predetermined distance from the incision point that it is recommended that the user deactivates apparatus 800. The markings 860 may be used to inform or alert the user when to deactivate apparatus 800 to stop applying plasma as tip 806 is pulled proximally toward the incision point. As tip 806 is pulled proximally toward the incision point, when a marking 860 becomes visible to the user, the user will know to deactivate apparatus 800. It is to be appreciated that tip 806 may include any number of markings, each at a predetermined distance from the distal end of tip 806. In some embodiments, different markings may correspond to different procedures or different generator settings.

Referring to FIG. 12H, in one embodiment, a trace card 880 may be used to further increase safety and ensure deactivation of apparatus 800 while tip 806 is at a predetermined minimum distance from an incision point through surface 890. Trace card 800 is configured in a semi-circular shape having curved or semi-circle edge 882, and linear edge 884, with a radius r and a diameter d (shown in FG. 12H). At the midpoint (i.e., the radius r of semi-circular card 880) of linear edge 884, a semi-circular edge 886 is cut out from edge 884. To use trace card 880, referring to FIGS. 12G, 12H, card 880 is placed on a tissue or skin surface 890 with the incision that tip 806 is going to be inserted into aligned with the center 888 (i.e., a point on the skin surface equidistance from all points on edge 886) of the space defined by semi-circular edge 886. In this position, a line following curved edge 882 is traced over the skin (e.g., using a marker or drawing or marking tool) to create a curved line on the skin surface 890. When tip 806 is inserted into the subdermal plane via the incision point in tissue surface 890 and activated, a glow will appear on the tissue surface 890 outside or past the bounds of the line traced using edge 882 when tip 806 has been inserted sufficiently far to be activated safely. As tip 806 is pulled proximally toward the incision point, the glow on the tissue surface 890 will approach the traced line made using edge 882 and when the glow 890 is within the bounds of traced line following edge 882, apparatus 800 should be shut off to prevent damage to tissue near the incision point and/or on the tissue surface. The radius r of semi-circular edge 882 is based on the distance from a marking 860 to the distal end of tip 806 or to the center of port 830.

In some embodiments, both trace card 880 and markings 860 may be employed with apparatus 800 to increase safety while using apparatus 800. While tip 806 is treating a subdermal plane, if the glow on the tissue surface 890 is within the bounds of a semi-circular line drawn using edge 882 of card 880 and/or a marking 860 becomes visible to the user, the user will know to deactivate apparatus 800.

It is to be appreciated that although in the embodiments above, tip 806 of apparatus 800 is coupled to shaft 804 by gluing tip 806 to support tube 850, the present disclosure contemplates other methods for securing tip 806 to shaft 804, such as, but not limited to, brazing, use of threads, combining tip 806 and tube 850 into a single piece, high temperature plastic over molding, etc.

Below, several methods or techniques for securing distal tip 806 to shaft 804 are described, where tube 850 has been removed from apparatus 800 and thus is not used for securing tip 806 to shaft 804.

For example, referring to FIGS. 121, 12J, 12K, a distal tip 1506 and the distal end of a shaft of an electrosurgical apparatus (e.g., including the features of apparatus 800) are shown in accordance with an embodiment of the present disclosure. Tip 1506 includes ports 1530A, 1530B and tabs or protrusions 1545A, 1545B, which extend away from the outer wall or exterior of tip 1506 and are disposed toward the proximal end of tip 1506. The distal end of shaft 1504 may include slots 1547A,15547B, which are aligned along axis 1570. In this embodiment, to attach tip 1506 to shaft 1504, the proximal end of tip 1506 is inserted into the distal end of shaft 1504. When the proximal end of tip 1506 is disposed through the distal end of shaft 1504, slot 1547A is configured to receive protrusion 1545A and slot 1547A is configured to receive protrusion 1545B to couple tip 1506 to shaft 1504. It is to be appreciated that the dimensions of slots 1547 and tabs 1545 are selected to require press fitting to insert tabs 1545 into slots 1547. Furthermore, the proximal end of each slot 1547 includes a generally circular end 1551 configured to accommodate the circumference of each circular tab 1545, such that each tab 1545 snaps into each corresponding circular end 1551. In one embodiment, the portion of each slot 1547 other than circular end 1551 is configured to have a width that is less than the diameter of each tab 1545. In this way, when tabs 1545 are disposed in circular ends 1551, tip 1506 cannot be decoupled from shaft 1504 absent pulling forces that exceed the forces normally exerted during procedures that the electrosurgical device is used in.

As another example, referring to FIGS. 12L, 12M, a distal tip 1606 and the distal end of a shaft 1604 of an electrosurgical apparatus (e.g., including the features of apparatus 800) are shown in accordance with an embodiment of the present disclosure.

Tip 1606 includes ports 1630A, 1630B and protrusion or tabs 1640A, 1640B, which extend away from the outer wall of tip 1606 and are disposed toward the proximal end of tip 1606. The distal end of shaft 1604 may include L-shaped slots 1642A, 1642B, each having a first portion aligned with axis 1670 and a second portion that is perpendicular to axis 1670. In this embodiment, to attach tip 1606 to shaft 1604, the proximal end of tip 1606 is inserted into the distal end of shaft 1604 with protrusion 1640A aligned with the first portion of slot 1642A and protrusion 16408 aligned with the first portion of slot 16428. When protrusion 1640A meets the end of the first portion of slot 1642A and protrusion 16408 meets the end of the first portion of slot 16428, tip 1606 is rotated about axis 1670 until protrusion 1640A reaches the end of the second portion of slot 1642A and protrusion 1640B reaches the end of the second portion of slot 1642B. In this position, each protrusion 1640 and second portion of slot 1642 prevent tip 1606 from pulled along axis 1670 distally to remove tip 1606 from shaft 1604. In one embodiment, slots 1642 include circular ends 1651 similar to circular ends 1551 described above.

As another example, referring to FIGS. 12N, 120, 12P, a distal tip 1706 and the distal end of a shaft 1704 of an electrosurgical apparatus (e.g., including the features of apparatus 800) are shown in accordance with an embodiment of the present disclosure. Tip 1706 includes ports 1730A, 1730B, a first portion 1748A having a diameter approximately equal to the outer diameter of shaft 1704 and a second portion 17488 having a diameter approximately equal to the inner diameter of the interior of shaft 1704. The diameter of portion 1748A is larger than the diameter of portion 1748B and portion 1748A is disposed toward the distal end of tip 1706 and portion 17488 is disposed toward the proximal end of tip 1706. Tip 1706 includes protrusions or tabs 1744A, 17448, which are disposed around the outer wall of portion 17488 (e.g., at diametrically opposed positions about axis 1770) and extend away from the outer wall of portion 1748B. As shown in FIG. 12P, each protrusion 1744 includes a ledge 1748A, which extends away from the outer wall of portion 17488 perpendicularly relative to axis 1770. Each protrusion 1744 also includes a slanted wall 1749B, which slants from the end of ledge 1749A furthest from the outer wall of portion 17488 to the outer wall of 17488.

As shown in FIG. 12P, proximately to the distal end of shaft 1704, shaft 1704 includes slots 1746A, 1746B disposed through the outer wall of shaft 1704 at diametrically opposed positions. When portion 1748B is disposed through the distal end of shaft 1704, protrusion 1744A is received by slot 1746A and protrusion 1744B is received by slot 1746B. Ledges 1748A, 1748B interact with slots 1748A, 1746B to prevent tip 1706 from being pulled away from shaft 1704, thus securely attaching tip 1706 to shaft 1704. Furthermore, portion 17488 of tip 1706 provides support to the connection between tip 1706 and the distal end of shaft 1704, thus eliminating the need for a support tube to support to the connection.

Referring to FIGS. 12Q, 12R, a distal tip 1806 and the distal end of a shaft 1804 of an electrosurgical apparatus (e.g., including the features of apparatus 800) are shown in accordance with an embodiment of the present disclosure. Tip 1806 a portion 1862 (e.g., made of ceramic) including ports 1830A, 1830B, where portion 1862 is coupled to shaft 804 using an over molded cap 860. In this embodiment, the distal end 1805 of shaft 1804 is stepped (i.e., having a smaller diameter than the remaining portion of shaft 804). Portion 1862 includes a proximal end and a distal end, where the proximal end receives the stepped distal end 1805 of shaft 1804. Electrode 1818 is disposed through shaft 1804, through portion 1862, and extends past the distal end of portion 1862. Cap 1860 is formed or molded (e.g., via injection molding using a suitable thermoplastic or polymeric material) over the distal end of electrode 1818 such that a stepped portion 1861 is disposed through the distal end of portion 1862 and portion 1862 is attached to shaft 1804. It is to be appreciated that cap 1860 is shaped in a blunt shape (e.g., without sharp edges) to enable tip 1806 to be easily inserted into subdermal planes of patient tissue during treatments.

Referring to FIGS. 12S, 12T, a distal tip 1906 and the distal end of a shaft 1904 of an electrosurgical apparatus (e.g., including the features of apparatus 800) are shown in accordance with an embodiment of the present disclosure. Tip 1906 is configured with a port 1930 that extends around the entire perimeter of the shaft 1904 and tip 1906. In this embodiment, electrode 1918 is configured to be rigid and the distal end of electrode 1918 is coupled to a cap 1972 (e.g., configured in a semi-hemispherical, blunt shape), where port 1930 is disposed between cap 1972 and the distal end of shaft 1904. In this embodiment, electrode 1918 is configured to mount cap 1972 a fixed distance from the distal end of shaft 1904. Since, in this embodiment, port 1930 extends around the entire perimeter of shaft 1904 and tip 1906, the treatment area of tip 1906 is increased greatly as shown in FIG. 12T, where tip 1906 is shown treating tissue 1981 disposed around various sides of tip 1906 with plasma.

It is to be appreciated that any of the distal tips and corresponding features shown in FIGS. 12A-12U and described above may be used as the distal tip for apparatus 600 described above and any of the distal tips and corresponding features shown in FIGS. 9A-11P and described above may be used as the distal tip for apparatus 800. Furthermore, any of the features of these embodiments may be mixed or rearranged to form new tips without deviating from the scope of the present disclosure.

It is to be appreciated that, in some embodiments, distal tips for electrosurgical apparatus, such as apparatuses 600, 800, may be designed to reduce the buildup of tissue (e.g., coagulated body fluids), debris, and other materials present during surgical procedures on the electrodes in the distal tips of the apparatuses.

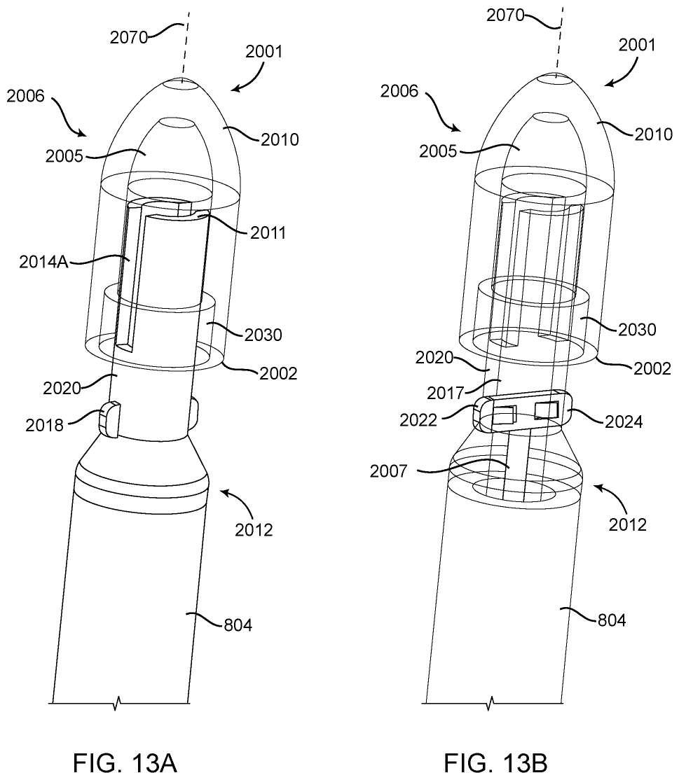

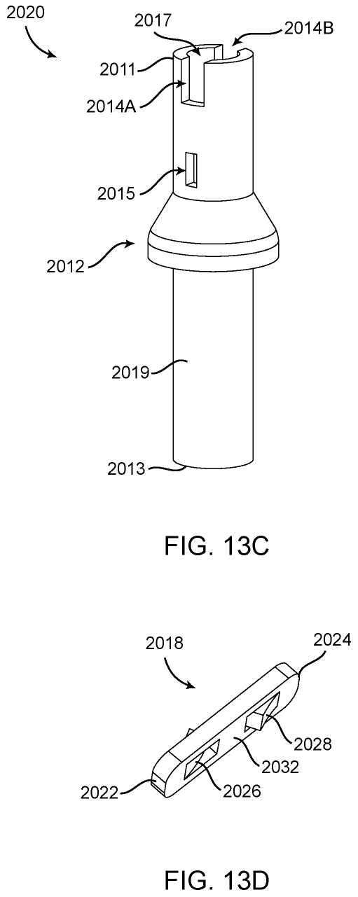

Referring to FIGS. 13A, 13B, a distal tip 2006 for use with an electrosurgical apparatus, such as apparatus 800, is shown coupled to shaft 804 in accordance with an embodiment of the present disclosure. Tip 2006 includes a cap or umbrella portion 2010 and a tube portion 2020. Furthermore, tip 2006 includes an electrode 2018. Cap 2010 includes a blunted, distal closed end 2001 and an open, proximal end 2002. Cap 2010 includes a hollow interior 2005, where, toward end 2002, interior 2005 includes a stepped, cylindrical slot 2030 embedded in the interior wall of cap 2010. Referring to FIG. 13C, tube 2020 includes a distal end 2011 and a proximal end 2013, where a hollow interior 2017 of tube 2020 extends from end 2013 to end 2011. Tube 2020 further includes a conic or frustoconical (i.e., a frustrum of a cone) portion 2012, slots 2014A, 2014B (formed on the distal end 2011 of tube 2020), slot 2015 and a slot (not shown) disposed in a diametrically opposite position to slot 2015 about axis 2070. Each of the slots in tube 2020 extend through the outer surface of tube 2020 providing access to the hollow interior 2017.

Referring to FIG. 13D, an electrode 2018 is provided for use with tube 2020 and tip 2006. Electrode 2018 includes sides or ends 2022, 2024, a surface 2032, and a surface (not shown) opposite to surface 2032. Electrode 2018 includes tabs 2026, 2028, where tab 2026 is biased to extend from the surface opposite to surface 2032 and tab 2028 is biased to extend from surface 2032. To mount electrode 2018 to tube 2020, tab 2026 is pressed toward the surface opposite to surface 2032 and tab 2028 is pressed toward surface 2032 and electrode 2018 is inserted through slot 2015 and the slot opposite to slot 2015 of tube 2020. In this position, each end 2022, 2024 of electrode 2018 extends from a respective slot 2015 (or slot opposite to slot 2015) of tube 2020, as best seen in FIGS. 13A, 13B. While electrode 2018 is mounted to tube 2020, tabs 2026, 2028 return to each of their respective biased positions (shown in FIG. 13D) and prevent electrode 2018 from being unmounted from tube 2020.

Referring to FIGS. 13A, 13B, 13C, a proximal portion 2019 of tube 2020 is disposed through the distal end of a shaft of an electrosurgical apparatus, such as shaft 804 of apparatus 800, until conic section 2012 is disposed against the distal end of shaft 804. It is to be appreciated that the diameter of the widest portion of conic section 2012 is substantially the same as the outer diameter of shaft 804 and the diameter of portion 2019 of tube 2020 is substantially the same as the inner diameter of shaft 804. A conductive wire extends through shaft 804 and through tube 2020, where the distal end 2007 of the conductive wire is coupled to electrode 2018 and the proximal end of the conductive wire is coupled to a power source (e.g., an electrosurgical generator) for providing electrosurgical energy to electrode 2018. Cap or umbrella 2010 is disposed over the distal end 2011 of tube 2020, such that distal end 2011 extends into and is coupled to the interior 2005 of cap 2010. The diameter of slot 2030 is greater than the diameter of the distal portion of tube 2020, such that, while cap 2010 is coupled to tube 2020, cylindrical slot 2030 forms a gas port. It is to be appreciated a portion of each slot 2014 is disposed in port 2030.

Inert gas provided from a gas source via shaft 804 flows through interior 2017 of tube 2020, through slots 2014 of distal end 2011, into and out of port 2030 around the perimeter of tube 2020 in a proximal direction along axis 2070. It is to be appreciated that the shape and design of cap 2010 is configured to direct the inert gas in the proximal direction. While electrode 2018 is energized and the inert gas exits port 2030, the gas is ionized by ends 2022, 2024 of electrode 2018 and plasma is generated around the perimeter of tube 2020 to treat tissue proximate to the exterior of tip 2006. The gas exiting port 2030 and the plasma generated flow in a proximal direction along axis 2070 and when the gas and generated plasma contacts conic section 2012, conic section 2012 causes (i.e., redirects some of) the gas and generated plasma to have a radial component relative to axis 2070 to further spread the gas and generated plasma radially away from tube 2020 and shaft 804 to treat tissue.

The design of tip 2006 provides several safety benefits and design efficiencies. First, as stated above, users are directed to activate an electrosurgical apparatus, such as electrosurgical apparatus 800, while the distal tip is inserted into tissue at a predetermined distance from the incision point in the tissue and the tip is moving in a proximal direction (i.e., in the direction that removes shaft and distal tip of the apparatus from the tissue). Since tip 2006, ejects inert gas in a proximal direction along axis 2070, the gas and plasma ejected do not treat tissue disposed distally to tip 2006 (which is undesired because it is outside the desired treatment area). Also, since the gas flows against the direction of movement of the tip, debris and coagulated tissue are prevented from entering port 2030 into interior 2005 of tip 2006. Second, since ends 2022, 2024 of electrode 2018 are disposed externally to tip 2006, coagulated tissue or other material buildup on electrode 2018 can be easily cleaned without requiring access to the interior of tip 2006. Third, the proximal portion of tube 2020 includes portion 2019, which is disposed in the distal end of shaft 804. Portion 2019 supports the junction or connection between the distal end of shaft 804 and tip 2006. Since portion 2019 is integrated in tip 2006, the design of tip 2006 does not require a support tube (e.g., such as support tube 650, described above) for structural support.

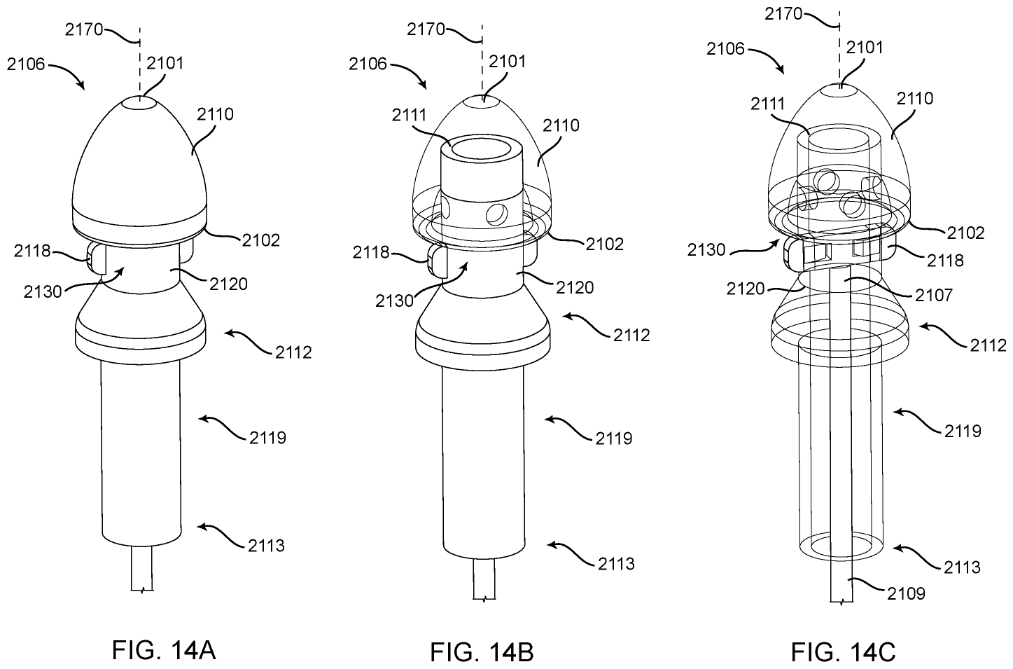

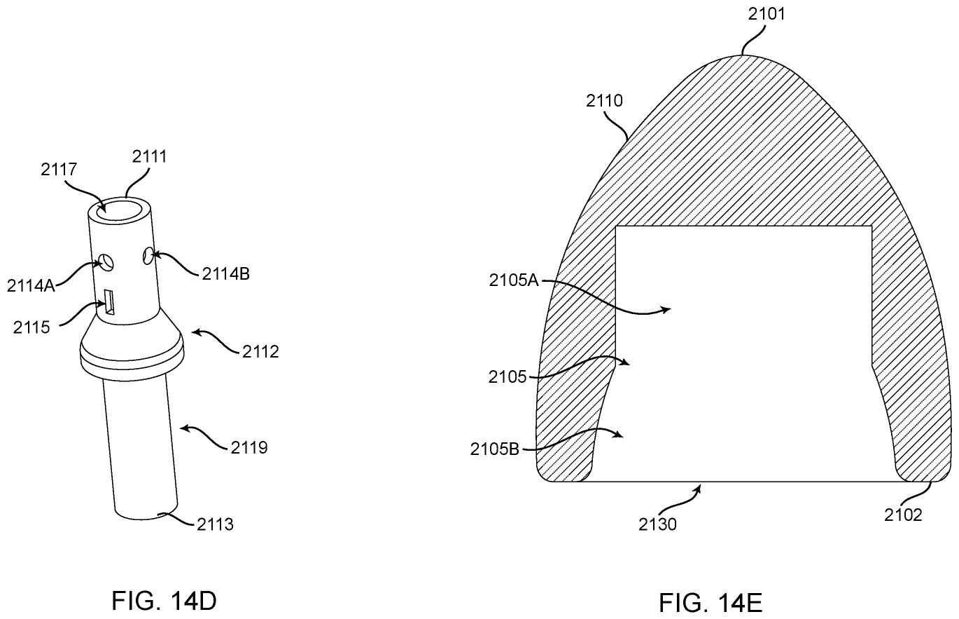

Referring to FIGS. 14A-14C, a distal tip 2106 for use with an electrosurgical apparatus, such as apparatus 800, is shown in accordance with an embodiment of the present disclosure. Tip 2106 includes a cap or umbrella portion 2110 and a tube portion 2120. Furthermore, tip 2106 includes an electrode 2118, where electrode 2118 includes the same features as electrode 2018, described above. Cap 2110 includes a blunted, distal closed end 2101 and an open, proximal end 2102. Referring to FIG. 14D, tube 2120 includes a distal end 2111 and a proximal end 2113, where a hollow interior 2117 of tube 2120 extends from end 2113 to end 2111. Tube 2120 further includes a conical or frustoconical portion 2112, apertures 2114, slot 2115 and a slot (not shown) disposed in a diametrically opposite position about axis 2170 to slot 2115. It is to be appreciated that tube 2120 may include any number of apertures spaced around a distal portion of tube 2120. In one embodiment, tube 2120 includes four apertures 2114 spaced equidistantly around the exterior of the distal portion of tube 2120. Electrode 2118 is mounted to tube 2120 in the manner described above with respect to electrode 2018 and tube 2020.

A conductive wire 2109 extends through the shaft that tip 2106 is coupled to and through tube 2120, where the distal end 2107 of the conductive wire 2109 is coupled to electrode 2118 and the proximal end of the conductive wire 2109 is coupled to a power source (e.g., an electrosurgical generator) for providing electrosurgical energy to electrode 2118. Cap or umbrella 2110 is disposed over the distal end 2111 of tube 2120, such that distal end 2111 extends into and is coupled to the interior of cap 2110. Referring to FIG. 14E, cap 2110 includes a hollow interior 2105 having a first portion 2105A and a second portion 21058. Portion 2105A is configured as a cylindrical slot with substantially the same diameter as the distal portion of tube 2120 to receive distal end 2111 of tube 2120. Portion 21058 is configured in a frustoconical shape and includes a larger diameter (throughout its entire length) than the distal portion of tube 2120 and slot 2105A. Distal end 2111 of tube 2120 is disposed in and coupled to slot 2105A to mount cap 2110 to tube 2120. In this position, apertures 2114 are disposed in portion 2105B.

The shape of portion 2105B is configured to provide gas provided via apertures 2114 out through port 2130 in a proximal direction. The proximal portion 2119 of tube 2120 is coupled to a shaft of an electrosurgical device, such as shaft 804, in the manner described above with respect to portion 2019 of tube 2020.

Inert gas is provided from a gas source via the shaft that tube 2120 is coupled to and flows through interior 2117 of tube 2120, through apertures 2114 of the distal portion of tube 2120, into the interior of cap 2110, and out of port 2130 around the perimeter of tube 2120 in a proximal direction along axis 2170. While electrode 2118 is energized and the inert gas exits port 2130, the gas is ionized by the ends of electrode 2118 protruding exterior to tube 2120 and plasma is generated around the perimeter of tube 2120 to treat tissue proximate to the exterior of tip 2120. The gas exiting port 2130 and the plasma generated flow in a proximal direction along axis 2170 and when the gas and generated plasma contact conic section 2112, conic section 2012 causes (i.e., redirects) the gas and generated plasma to have a radial component relative to axis 2070 to further spread the gas and generated plasma radially away from tube 2120 and the shaft tip 2106 is coupled to (e.g., shaft 804) to treat tissue.

Referring to FIGS. 15A-15C, a distal tip 2206 for use with an electrosurgical apparatus, such as apparatus 800, is shown in accordance with an embodiment of the present disclosure. Tip 2206 includes a cap or umbrella portion 2210 and a tube portion 2220. Furthermore, tip 2206 includes an electrode 2218. Referring to FIG. 15E, electrode 2218 is configured in a substantially tubular shape with, in one embodiment, pointed ends.

As shown in FIGS. 15A-15C, cap 2210 includes a blunted, distal closed end 2201 and an open, proximal end 2202. Cap 2210 includes a hollow interior and is configured with the same features as cap 2110 described above. Referring to FIG. 15D, tube 2220 includes a distal end 2211 and a proximal end 2213, where a hollow interior 2217 of tube 2220 extends from end 2213 to end 2211. Tube 2210 further includes a conical or frustoconical portion 2212, apertures 2214, aperture 2215 and an aperture (not shown) disposed in a diametrically opposite position to aperture 2215. It is to be appreciated that tube 2220 may include any number of apertures spaced around a distal portion of tube 2220. In one embodiment, tube 2220 includes four apertures 2214 spaced equidistantly around the exterior of the distal portion of tube 2220. Electrode 2218 is mounted to tube 2220 by inserting electrode 2218 through aperture 2215 and the aperture opposite aperture 2215, such that the ends of electrode 2218 extend past the outer wall of tube 2220. Tube 2220 is coupled to a shaft of an electrosurgical device, such as shaft 804, in the manner described above with respect to tube 2020.

A conductive wire 2209 extends through the shaft and through tube 2220, where the distal end 2207 (best seen in FIG. 15C) of the conductive wire 2209 is coupled to electrode 2218 and the proximal end of the conductive wire 2209 is coupled to a power source (e.g., an electrosurgical generator) for providing electrosurgical energy to electrode 2218. Cap or umbrella 2210 is disposed over the distal end 2211 of tube 2220, such that distal end 2211 extends into and is coupled to the interior of cap 2210 and the proximal end 2202 of cap 2210 forms a port 2230. It is to be appreciated that cap 2210 is coupled to tube 2220 in the manner described above with respect to cap 2110 and tube 2120.

Inert gas is provided from a gas source via the shaft that tube 2220 is coupled to and flows through interior 2217 of tube 2220, through apertures 2214 of the distal portion of tube 2220, into the interior of cap 2210, and out of port 2230 around the perimeter of tube 2220 in a proximal direction along axis 2270. While electrode 2218 is energized and the inert gas exits port 2230, the gas is ionized by the ends of electrode 2218 protruding exterior to tube 2220 and plasma is generated around the perimeter of tube 2220 to treat tissue proximate to the exterior of tip 2220. The gas exiting port 2230 and the plasma generated flow in a proximal direction along axis 2270 and when the gas and generated plasma contact conic section 2212, conic section 2212 causes (i.e., redirects) the gas and generated plasma to have a radial component relative to axis 2270 to further spread the gas and generated plasma radially away from tube 2220 and the shaft to treat tissue.

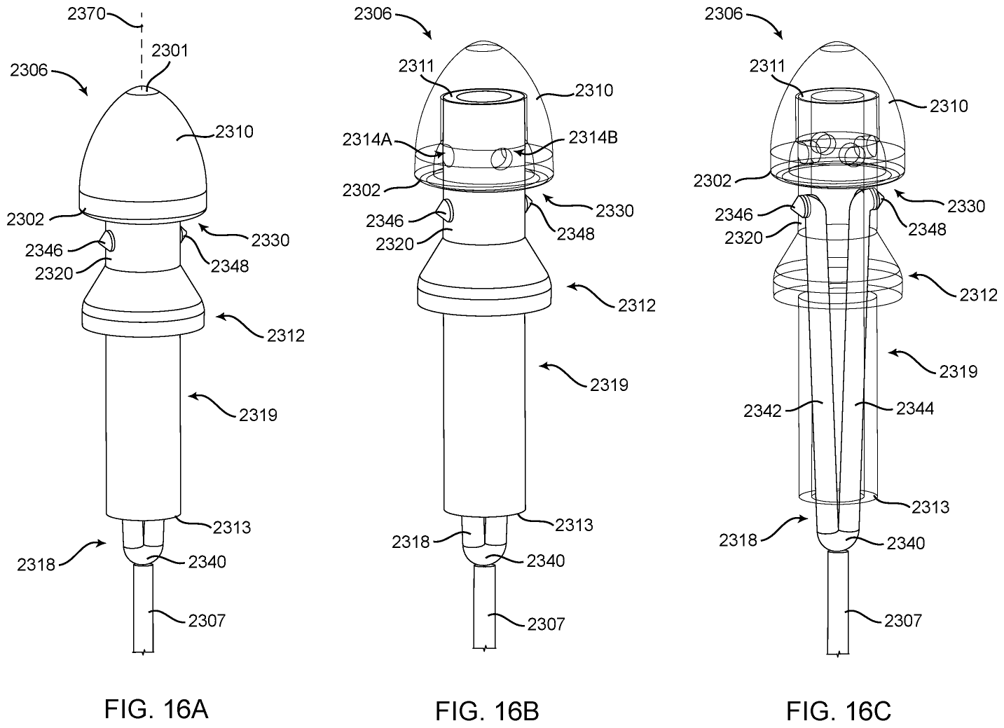

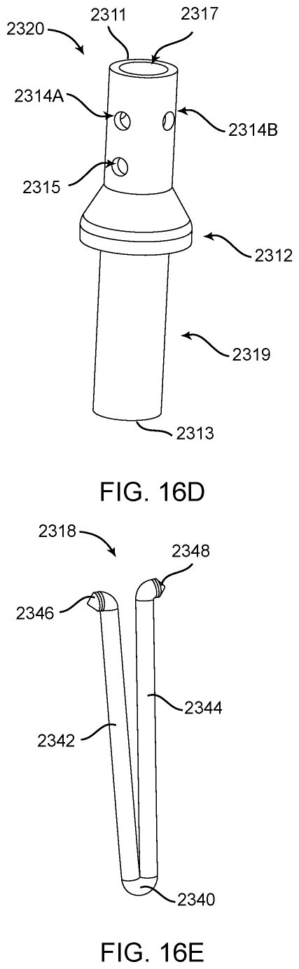

Referring to FIGS. 16A-16C, a distal tip 2306 for use with an electrosurgical apparatus, such as apparatus 800, is shown in accordance with an embodiment of the present disclosure. Tip 2306 includes a cap or umbrella portion 2310 and a tube portion 2320. Furthermore, tip 2306 includes an electrode 2318. Referring to FIG. 16E, electrode 2318 is configured in a tong-like shape. Electrode 2318 is bent about a proximal end 2340 and includes distal bent ends 2346, 2348, which extend from and are bent relative to rod portions 2342, 2344, respectively. Referring again to FIG. 16A-C, cap 2310 includes a blunted, distal closed end 2301 and an open, proximal end 2302. Cap 2310 includes a hollow interior and is configured with the same features as cap 2110 described above.

Referring to FIG. 16D, tube 2320 includes a distal end 2311 and a proximal end 2313, where a hollow interior 2317 of tube 2320 extends from end 2313 to end 2311. Tube 2310 further includes a conical or frustoconical portion 2312, apertures 2314, aperture 2315 and an aperture (not shown) disposed in a diametrically opposite position to aperture 2315. It is to be appreciated that tube 2320 may include any number of apertures spaced around a distal portion of tube 2320. In one embodiment, tube 2320 includes four apertures 2330 spaced equidistantly around the exterior of the distal portion of tube 2320 about axis 2370.

Referring to FIG. 16C, electrode 2318 is mounted to tube 2320 by bringing ends 2346, 2348 together and inserting ends 2346, 2348 into interior 2317 of tube 2320 via end 2313 until end 2346 reaches and is inserted through aperture 2315 and end 2348 reaches and is inserted through the aperture opposite to aperture 2315. In this position, ends 2346, 2348 protrude or extend past the outer wall of tube 2320 and electrode 2318 is secured to tube 2320. Tube 2320 is coupled to a shaft of an electrosurgical device, such as shaft 804, in the manner described above with respect to tube 2020.

A conductive wire extends through the shaft and through tube 2320, where the distal end 2307 of the conductive wire is coupled to electrode 2318 and the proximal end of the conductive wire 2309 is coupled to a power source (e.g., an electrosurgical generator) for providing electrosurgical energy to electrode 2318. Cap or umbrella 2310 is disposed over the distal end 2311 of tube 2320, such that distal end 2311 extends into and is coupled to the interior of cap 2310 and the proximal end 2302 of cap 2310 forms a port 2330. It is to be appreciated that cap 2310 is coupled to tube 2320 in the manner described above with respect to cap 2110 and tube 2120.

Inert gas provided from a gas source via the shaft tube 2320 is coupled to flows through interior 2317 of tube 2320, through apertures 2314 of the distal portion of tube 2320, into the interior of cap 2310, and out of port 2330 around the perimeter of tube 2320 in a proximal direction along axis 2370. While electrode 2318 is energized and the inert gas exits port 2330, the gas is ionized by the ends 2346, 2348 of electrode 2318 protruding exterior to tube 2320 and plasma is generated around the perimeter of tube 2320 to treat tissue proximate to the exterior of tip 2330. The gas exiting port 2330 and the plasma generated flow in a proximal direction along axis 2370 and when the gas and generated plasma contact conic section 2312, conic section 2012 causes (i.e., redirects) the gas and generated plasma to have a radial component relative to axis 2370 to further spread the gas and generated plasma radially away from tube 2320 and the shaft to treat tissue.

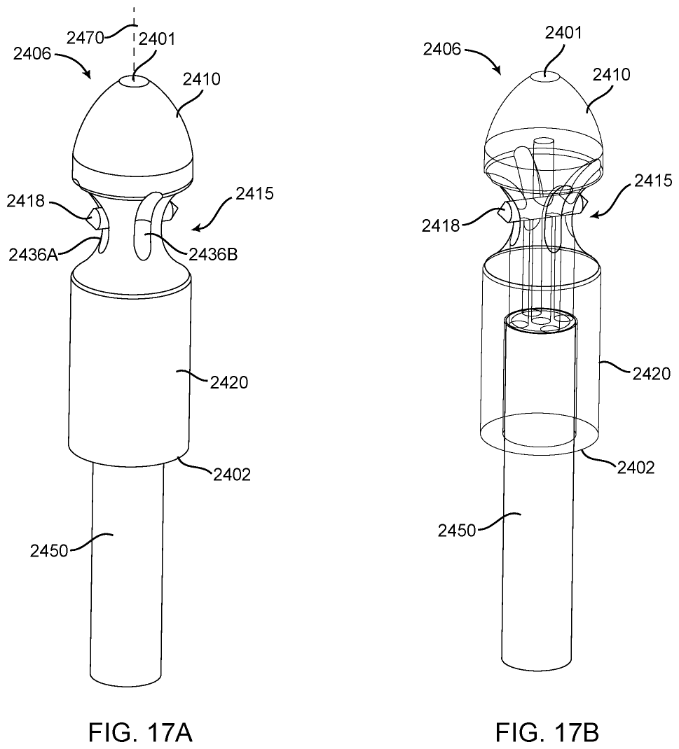

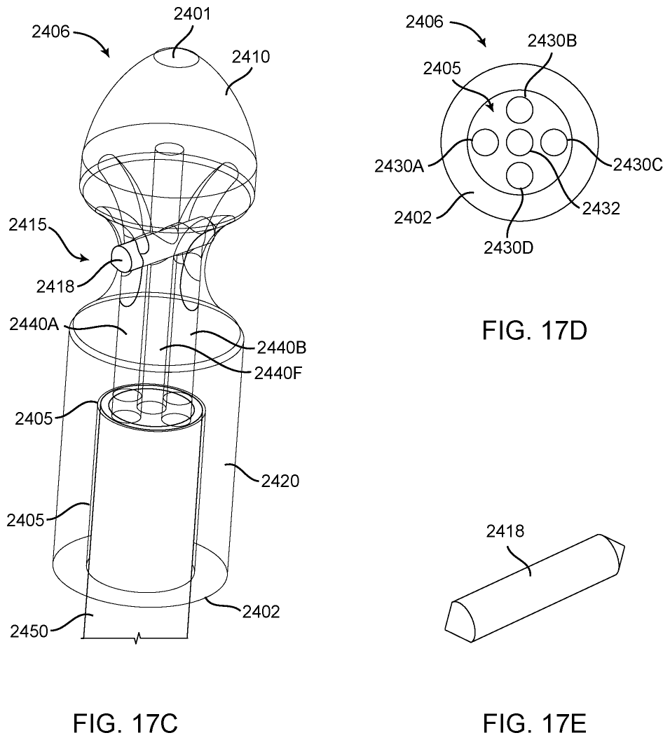

Referring to FIGS. 17A-17D, a distal tip 2406 for use with an electrosurgical apparatus, such as apparatus 800, is shown in accordance with an embodiment of the present disclosure. Tip 2406 includes a closed, blunted distal end 2401, and an open, proximal end 2402. Tip 2406 further includes a cap portion 2410 (shaped externally in a similar manner to cap 2110, described above), an hourglass-shaped or hyperboloid portion 2415, and a cylindrical portion 2402. End 2402 of tip 2406 includes an opening revealing a cylindrically shaped slot 2405 for receive a distal portion of a support tube 2450 used to couple tip 2406 to a shaft of an electrosurgical apparatus, such as shaft 804, in the manner described above with respect to other distal tips for use with an electrosurgical apparatuses 600, 800.

As best seen in FIG. 17D, where a view through the proximal end 2402 of tip 2406 is shown, tip 2406 includes a plurality of channels or ports 2430A-D and a wire channel 2432. Ports 2430 extend distally from the distal end 2407 of slot 2405 and terminate at respective arced openings 2436 in the outer wall of tip 2406 in the hourglass-shaped portion 2415, where each opening 2436 follows the hourglass shape of portion 2415. The openings 2436 in portion 2415 may be equidistantly spaced around the perimeter of portion 2415 about axis 2470. Although not shown, a channel that traverses axis 2470 connects port 2430A and port 2403C (i.e., at diametrically opposed positions about axis 2470). In one embodiment, an electrode 2418, show in FIG. 17E, and configured in the same shape as electrode 2218, described above, is used with tip 2406. In this embodiment, electrode 2218 is disposed through the channel that connects ports 2430A and 2430C such that each end of electrode 2218 extends to the exterior of tip 2406 past openings 2436. It is to be appreciated that electrode 2418 does not fully cover ports 2430A, 2430C, thus gas may still flow over electrode 2418.

A distal end of a conductive wire extending through tube 2450 and the shaft tip 2406 is disposed through wire channel 2432 and coupled to electrode 2418 and a proximal end of the conductive wire is coupled to a power source for receiving electrosurgical energy. In this way, when inert gas is provided via the shaft to the interior of support tube 2450, the inert gas flows through ports 2430 and out through each of the openings 2436. In the case of openings 2436 for ports 2430A, 2430C, gas flows over electrode 2418. When electrode 2418 is energized, plasma is generated outside of hourglass-shaped portion 2415 of tip 2406. The hourglass shape of portion 2415 enables less turbulence for gas flowing and exiting openings 2436 of each port 2430 and directs gas and plasma in an umbrella shape out of each opening 2436 to increase the treatment area.

It is to be appreciated that since the plasma is generated by the exposed ends of electrode 2418, any material (e.g., coagulated tissue) buildup during procedures on the ends of electrode 2418 are easily cleanable.

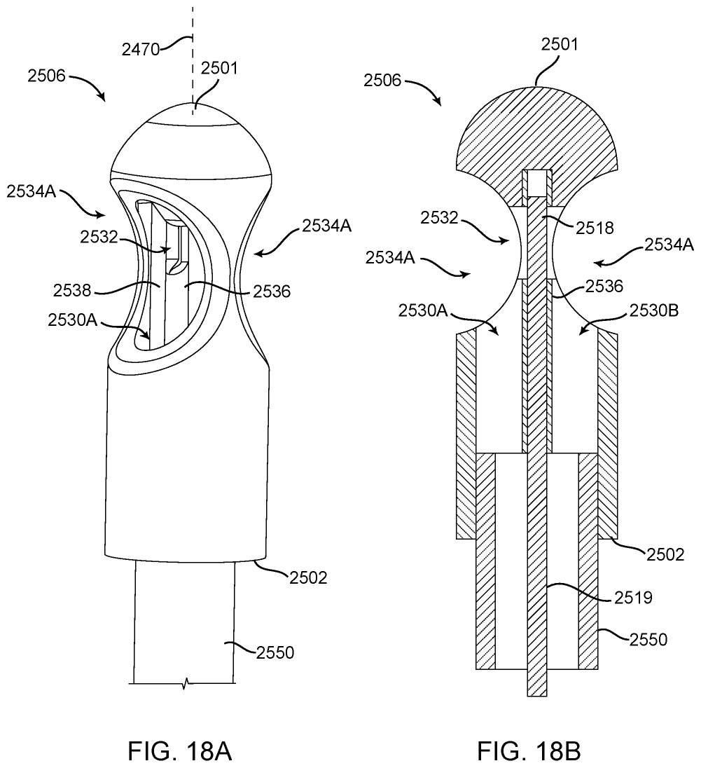

Referring to FIGS. 18A-18B, a distal tip 2506 for use with an electrosurgical apparatus, such as apparatus 800, is shown in accordance with an embodiment of the present disclosure. Tip 2506 includes a closed, blunted distal end 2501, and an open, proximal end 2502. Tip 2506 further includes concavely curved openings 2534A, 2534B, which are diametrically opposed about axis 2570 and reveal a partition 2538, which extends along axis 2570 within tip 2506 and splits the interior of tip 2506 into two portions forming ports 2530A, 2550B. Partition 2538 includes a tubular portion 2536 extending along axis 2570, where portion 2536 includes a hollow interior configured to receive a conductive wire 2519 including a distal end 2518 such that wire 2519 is embedded in the partition 2538. The distal end of tubular member 2536 reveals an opening or aperture 2532 through partition 2532 and tubular member 2536, such that the distal end 2518 of wire 2519 is exposed on either side of partition 2538 and mounted in openings 2534A, 2534B. It is to be appreciated that aperture 2532 is disposed at a predetermined distance from ports 2530A, 2530B.

The open proximal end 2502 of tip 2506 is configured to receive the distal portion of a support tube 2550 to couple tip 2506 to a shaft of an electrosurgical apparatus, such as shaft 804 of apparatus 800, in the manner described above with respect to support tube 650. The wire 2519 extends through tube 2550 and the shaft and the proximal end wire 2519 is coupled to a power source to provide electrosurgical energy to end 2518 of wire 2519, thus enabling end 2518 to function as an electrode. Inert gas provided via the shaft tip 2506 flows through the interior of tube 2550 and tip 2506 and is split by partition 2538. The inert gas flows on either side of partition 2538 and is provided in a distal direction via ports 2530A, 2530B to openings 2534A, 2534B, where electrode 2518 ionized the inert gas to form plasma when wire 2519 is energized. The curved shape of openings 2534A, 2534B impart a radial component to the plasma and inert gas to treat tissue around openings 2534A, 2534B. It is to be appreciated that, because electrode 2518 is disposed at the predetermined distance from ports 2340A, 2340B, material buildup on electrode 2518 (e.g., coagulated tissue) is prevented from entering the interior of tip 2506 via ports 2530A, 2530B.

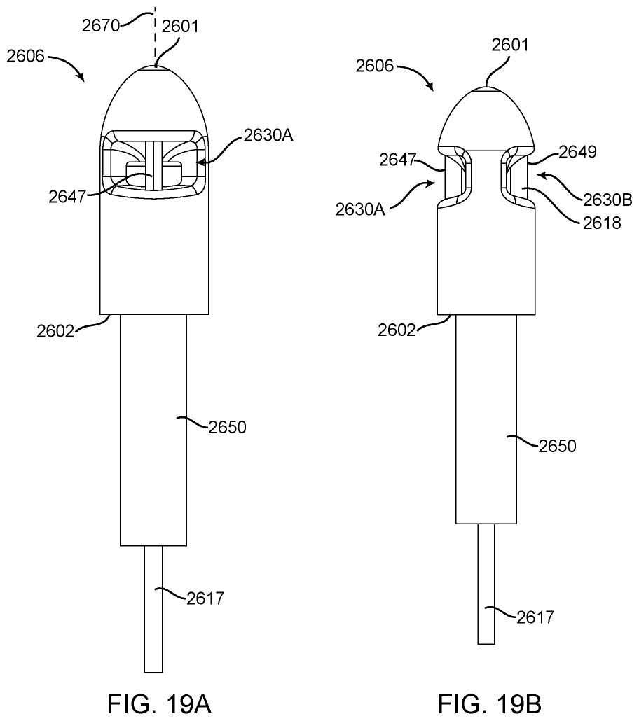

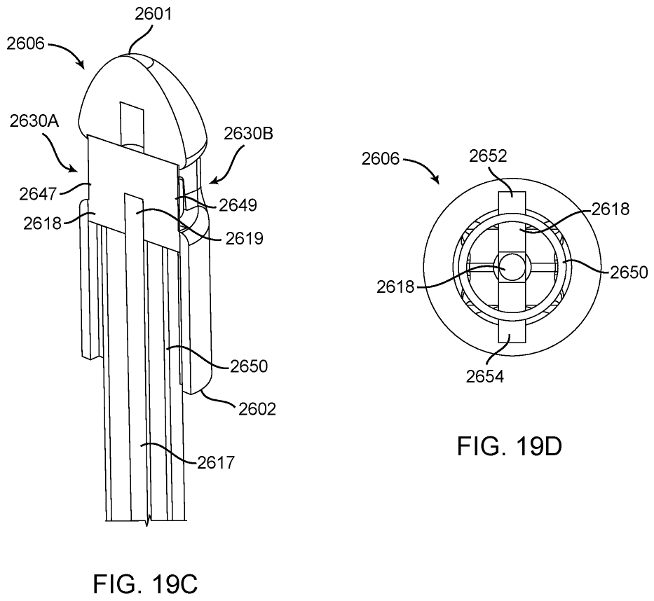

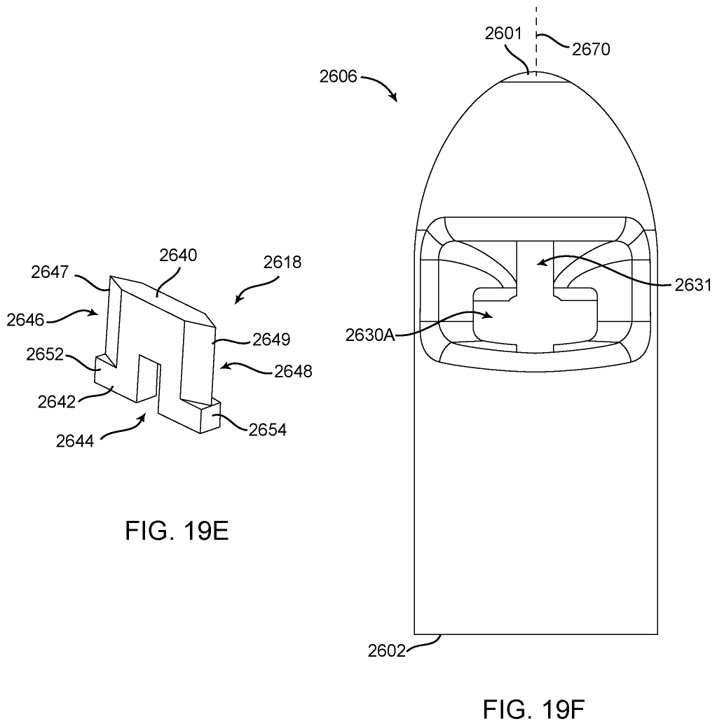

Referring to FIGS. 19A, 19B, 19C, a distal tip 2606 for use with an electrosurgical apparatus, such as apparatus 800, is shown in accordance with an embodiment of the present disclosure. Tip 2606 includes blunted, closed distal end 2601, open proximal end 2602, ports 2630A, 2630B, and electrode 2618. Tip 2606 is shaped in a similar manner to tip 606 described above and shown in FIGS. 10A-10G. Referring to FIG. 19F, tip 2606 includes an electrode slot 2631, which extends from port 2630A to port 2630B and is aligned along axis 2670. Slot 2631 is configured to receive electrode 1618 to mount electrode 1618 in and between ports 2630. Referring to FIG. 19E, electrode 2618 includes a distal end 2640, a proximal end 2642, side 2646, side 2648. Side 2646 includes a sharpened or beveled edge 2647 and a leg or mounting member 2652. Side 2648 includes a sharpened or beveled edge 2649 and a leg or mounting member 2654. Proximal end 2642 includes a slot 2644. As best seen in FIGS. 19A, 19B, 19C, slot 2631 is configured to receive electrode 2618, such that mounting members 2652, 2654 of electrode 618 are disposed on respective sides of slot 2631, such that edge 2647 extends into portion 2630A and edge 2649 extends into port 2630B.

As best seen in FIG. 19C, a cross-section of tip 2606, electrode 1618, a coupling tube 2650, and a conductive wire 1617 are shown, coupling tube 2650 (e.g., similar to tubes 650, 850 described above) is used to couple tip 2606 to the distal end of a shaft of an electrosurgical apparatus, such as shaft 804 of apparatus 800. The distal end of tube 2650 is inserted through the open proximal end 2602 of tip 2606 and coupled to the interior of tip 2606. The proximal end of tube 2650 is disposed through the distal end of a shaft, such as shaft 804, and coupled to the interior of the shaft. Conducting wire 2617 includes a proximal end (not shown) and a distal end 2619. The proximal end of electrode 2618 is coupled to a power source (e.g., an electrosurgical unit) for receiving electrosurgical energy and the distal end 2619 of electrode 2618 is disposed through the interior of tip 2806 and received by slot 2644. In this way, conductive wire 2617 provides electrosurgical energy to electrode 2618.

It is to be appreciated that since edges 2647, 2649 are disposed at a distance from the center (e.g., where end 2619 of wire 2617 is between ports 2630A, 2630B) of the interior of tip 2606 and proximately to ports 2630A, 2630B, coagulated fluid buildup during procedures entering ports 2630A, 2630B does not prevent (or is made more difficult to prevent) electrode 2618 from functioning, since edges 2647, 2649 are more proximate to tissue being treated. Any build up o edges 2647, 2649 is also easier to clean, since edges 2647, 2649 are disposed proximately to the exterior of tip 2606 and are accessible via ports 2630A, 2630B. Furthermore, the sharp edges 2647, 2649 of electrode 2618 concentrate energy provided to electrode 2618 to a small surface area (i.e., of edges 2647, 2649), and thus, combined with the proximity of edges 2647, 2649 to tissue makes it easier for the energy to be provided to tissue from electrode 2618 via plasma generated.

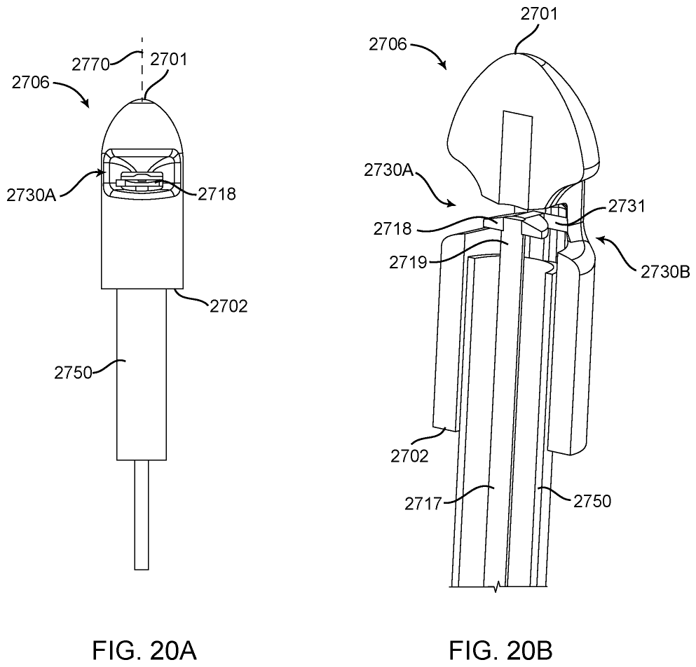

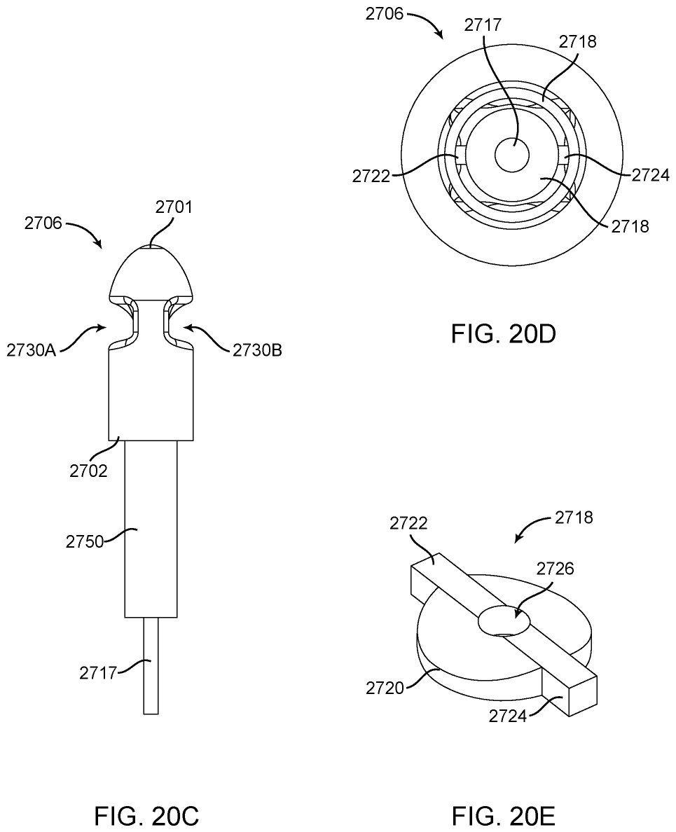

Although electrode 2618 is shown in FIGS. 19A, 19B, 19C as being mounted along axis 2670 (shown in FIG. 19A), in other embodiments, electrode 2618 may be mounted perpendicularly or traverse to axis 2670. For example, referring to FIGS. 20A-20D a tip 2706 for use with an electrosurgical apparatus, such as apparatus 800, is shown in accordance with an embodiment of the present disclosure. Tip 2706 includes distal end 2701, proximal end 2702, and ports 2730A, 2730B, where the shape of tip 2706 is configured in the same manner as tip 2606 described above. Furthermore, tip 2706 is coupled to a shaft of an electrosurgical apparatus, such as shaft 804, using a coupling tube 2750, in the manner described above with respect to tube 2560 and tip 2606.

Referring to FIG. 20E, an electrode 2718 is shown for use with tip 2706. Electrode includes a disk portion 2720 and mounting members 2722, 2724, which extend from the circumference of disc 2720 in opposite directions from diametrically opposed positions. An aperture or slot 2726 is disposed though the center of disc 2720.

Referring to FIGS. 20B, 20D, where FIG. 20B is a cross-section view of tip 2706 and FIG. 20D is a view through the proximal end 2702 of tip 2706, mounting members 2722, 2724 are disposed in respective mounting slots (e.g., where one slot 2731 is shown in FIG. 20B and the other slot is disposed at a diametrically opposed potion to slot 2731 about axis 2770) embedded in the inner walls of the interior of tip 2706. From this mounted position, the distal end 2719 of a conducting wire 2717 is received by slot 2726 to couple electrode 2718 to a power source. It is to be appreciated that because the circumference of disc 2720 extend into ports 2730A, 2730B, and the circumference of disc 2720 is tapered, electrode 2717 offers similar benefits to those described above with respect to electrode 2618.

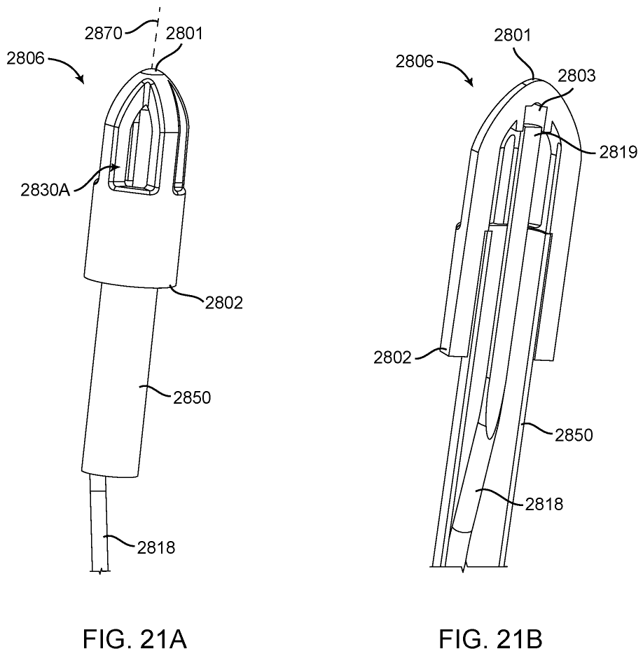

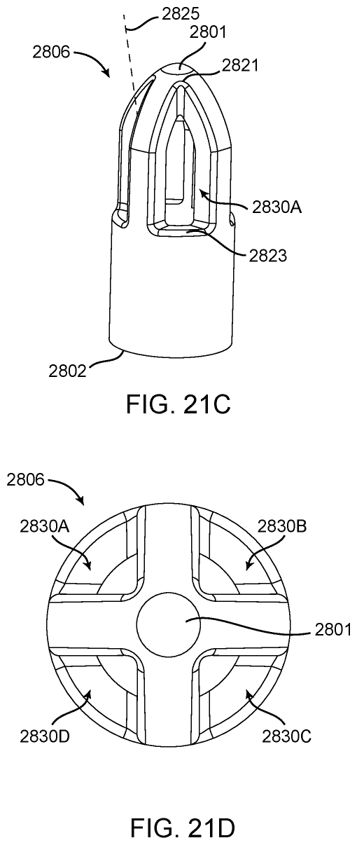

It is to be appreciated that, in some embodiments, the distal tip of an electrosurgical apparatus, such as apparatus 800, may include more than two ports. For example, referring to FIGS. 21A-21D, a tip 2806 is shown including four ports 2830A-D in accordance with an embodiment of the present disclosure. Tip 2806 includes a blunted, closed distal end 2801 and an open, circular, proximal end 2802. A coupling tube 2850 (e.g., similar to tubes 650, 850 described above) is used to couple tip 2806 to the distal end of a shaft of an electrosurgical apparatus, such as shaft 804 of apparatus 800. The distal end of tube 2850 is inserted through the open proximal end 2802 of tip 2806 and coupled to the interior of tip 2806. The proximal end of tube 2850 is disposed through the distal end of a shaft, such as shaft 804, and coupled to the interior of the shaft. An electrode 2818 is disposed through the shaft and tube 2850 and into the interior of tip 2806. As best seen in the cross-sectional view of tip 2806, tube 2850, and electrode 2818, in one embodiment, electrode 2818 is configured as a conducting wire having a proximal end (not shown) and a distal end 2819. The proximal end of electrode 2818 is coupled to a power source (e.g., an electrosurgical unit) for receiving electrosurgical energy and the distal end 2819 of electrode 2818 is disposed through the interior of tip 2806. In one embodiment, tip 2806 includes a slot 2803 configured to receive the proximal end 2819 of electrode 2818 to couple end 2819 thereto.

As best seen in FIGS. 21C, 21 D, tip 2806 includes four ports 2830A-D for ejecting inert gas provided to the tip 2806 and plasma generated when electrode 2818 is energized. Ports 2830 are equidistantly spaced about the perimeter of tip 2806. In one embodiment, ports 2830 are configured in an elongated shape extending along axis 2870 along the length of tube 2806. In the embodiment shown in FIGS. 21A-C, each port 2830 has a predetermined length. In one embodiment, the predetermined length is approximately 50% of the length of tip 2806, where a distal end 2821 of each port 2830 is disposed proximately to distal end 2401 of tip 2806 and a proximal end 2802 of each port 2830 is disposed approximately equidistantly from ends 2801, 2802 of tip 2806. The elongated shape and length of each port 2830 enable an elongated cleaning tool (e.g., including bristles) to be inserted through a port 2830 at an angle (as denoted by the dotted line 2825 in FIG. 21C), such that the cleaning tool is enabled to clean the interior of tip 2806, the interior of tube 2850, and electrode 2818. In this way, any tissue, debris, or other material buildup accumulated during procedures performed using tip 2806 may be more easily accessed and cleaned.

In use, when inert gas is provided to tip 2806 (e.g., via a shaft tip 2806 is coupled to) and electrode 2818 is energized, the inert gas is ionized to generated plasma, which is ejected from ports 2830 to treat tissue during a procedure.

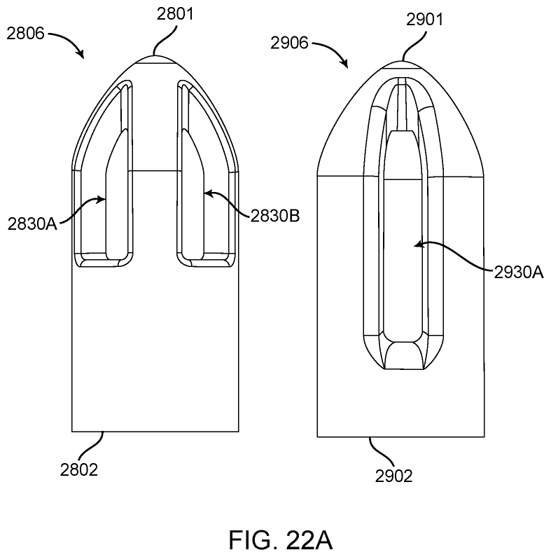

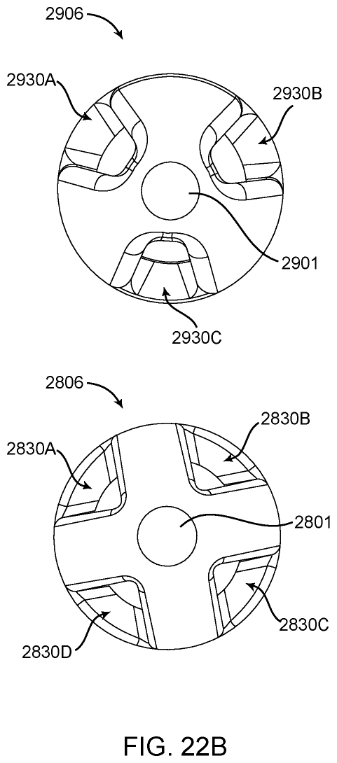

It is to be appreciated that, although tip 2806 includes four elongated ports 2830, in other embodiments, the ports 2830 of tip 2806 may include three ports and/or ports that are of different lengths. For example, referring to FIGS. 22A, 22B, tip 2806 is shown compared to distal tip 2906 for use with an electrosurgical apparatus, such as apparatus 800. Tip 2906 includes ports 2930A, 2930B, 2930C, which are equidistantly spaced from one another about the perimeter of tip 2906. In one embodiment, ports 2930, extend nearly (e.g., 80%-85%) the entire length of tip 2906 from distal end 2901 to proximal end 2902. As stated above, the elongated shape of ports 2930 enable a cleaning device to be inserted through one of ports 2930 to clean the interior of tip 2906, an electrode (e.g., electrode 2818) disposed in tip 2906, and/or the interior of a shaft/tube proximal end 2902 of tip 2906 is coupled to.

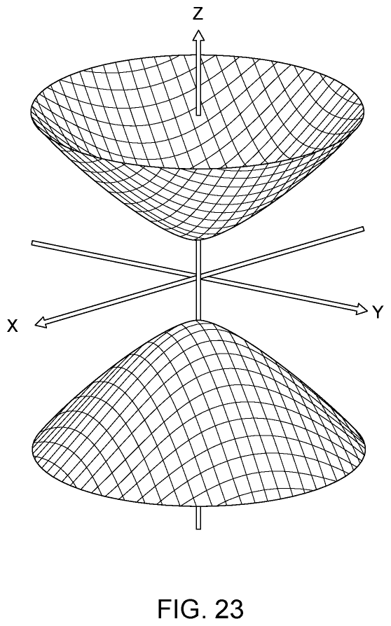

Referring to FIG. 23, the effective treatment area (e.g., a 360° treatment area) for any of the distal tips (e.g., 608) including two or more ports (e.g., 630A, 630B) described above is shown in accordance with the present disclosure. It is to be appreciated that if a shaft 604, 804 of the apparatus 600, 800 is positioned along the x-axis shown in FIG. 22, rotation the shaft 604, 804 would lead to increasing the treatment area shown.

Apparatuses 100, 200, 300, 600 and/or 800 and any of the distal tips described above, when used with an electrosurgical generator and a gas supply, are configured for use in cutting, coagulation, and/or ablation of soft tissue. When helium or another inert gas is passed over the energized electrode, such as electrode 618, 818, a helium plasma is generated which allows heat to be applied to tissue in two different and distinct ways. First, heat is generated by the actual production of the plasma beam (e.g., exiting ports 630, 830) itself through the ionization and rapid neutralization of the helium atoms. Second, since plasmas are very good electrical conductors, a portion of the RF energy used to energize the electrode and generate the plasma passes from the electrode to the patient and heats tissue by passing current through the resistance of the tissue, a process known as Joule heating. These two sources of tissue heating give the system and electrosurgical apparatuses of the present disclosure some very unique advantages during use as a surgical tool for the coagulation of subcutaneous soft tissue for the purpose of soft tissue contraction. These advantages are discussed in more detail below.

Some devices commercially available for subcutaneous soft tissue coagulation work on the principle of bulk tissue heating. In these devices, the energy is primarily directed into the dermis and the device is activated until a pre-set subdermal temperature in the range of 65° C. is achieved and maintained across the entire volume of tissue. As discussed above, at 65° C., the tissue being treated must be maintained at that temperature for greater than 120 seconds for maximal contraction to occur. Although these devices may be effective in achieving soft tissue contraction, the process of heating all of the tissue to the treatment temperature and maintaining that temperature for extended periods can be time consuming. In addition, during this process, the heat eventually conducts to the epidermis requiring constant monitoring of epidermal temperatures to ensure they do not exceed safe levels.

In contrast to previous approaches, the electrosurgical apparatuses 100, 200, 300, 600, 800 and electrosurgical generators of the present disclosure achieve soft tissue coagulation and contraction by rapidly heating the treatment site to temperatures greater than 85° C. for between 0.040 and 0.080 seconds. It is to be appreciated that electrosurgical apparatuses 100, 200, 300, 600, 800 and/or an electrosurgical generator coupled to the electrosurgical apparatuses 100, 200, 300, 600, 800 may include a processor configured to ensure the heat (provided via the tip of the applicator, e.g., tip 606, 806) applied to patient is maintained for between 0.040 and 0.080 seconds. For example, when button 616 of apparatus 600 or button 816 of apparatus 800 is pressed, a processor in applicator 600, 800 or in an electrosurgical generator coupled to applicator 600, 800 may be configured to apply electrosurgical energy to electrode 618, 818 continuously for between 0.040 and 0.080 seconds.

In some embodiments, a temperature sensor (e.g., an optical sensor) may be included in the distal tip (e.g., 606, 808) or be otherwise in communication with the apparatus 600, 800 and/or the electrosurgical generator. The temperature sensor provides temperature readings of the target tissue to the processor. The processor is configured to adjust the power outputted by the electrosurgical generator and the time duration that the heat is applied to the target tissue to ensure that temperatures greater than 85° C. for between 0.040 and 0.080 seconds are reached.

As will be described in greater detail below, in some embodiments, a predetermined power curve is applied to the electrode 618 of the apparatus 600 or electrode 818 of apparatus 800 by the electrosurgical generator that ensures the tissue is heated to temperatures greater than 85° C. for between 0.040 and 0.080 seconds. Furthermore, in accordance with the present disclosure, other properties associated with the application of plasma may be controlled to guarantee the temperatures of the tissue heated. For example, as will be described below, the flow rate of the inert gas provided to distal tip 606 of the apparatus 600 or distal tip 808 of apparatus 800 and the speed that the tip 606 or 806 is moved through the tissue plane may be selected to ensure the target temperatures described above are reached.

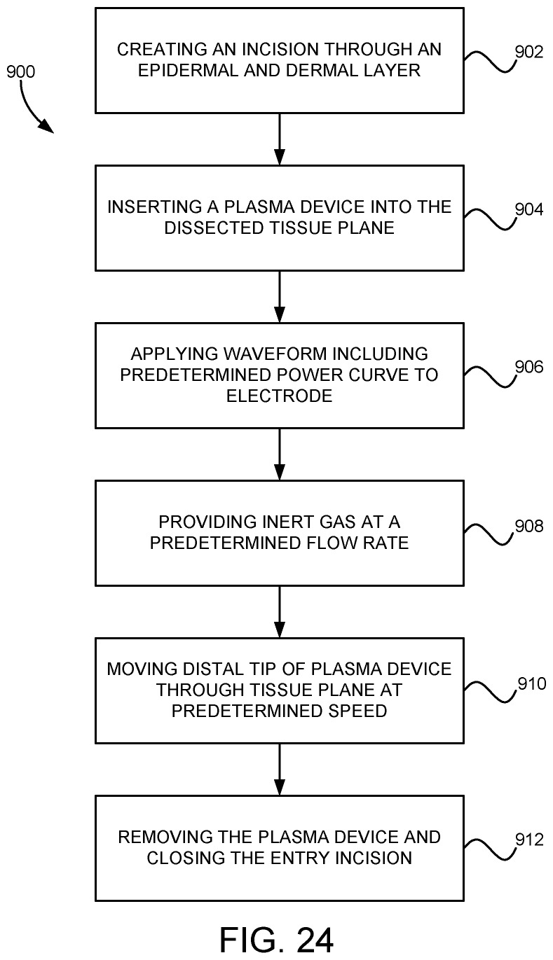

A method 900 of coagulating a subcutaneous layer of tissue will now be described in relation to FIG. 7 and FIG. 24. It is to be appreciated that method may be employed with any of the handpieces or plasma generators described above, for example, plasma generators 14, 100, 200, 300, 600, 800.

Initially, in step 902, an incision, i.e., an entry incision, is created through the epidermal 413 and dermal 411 layers of a patient at a location appropriate for a particular procedure. In step 904, the tip of the plasma generator is inserted into the dissected tissue plane. Next, the plasma generator 100, 200, 300, 600, 800 is activated to coagulate and/or ablate tissue to create a desired effect, e.g., (i) tighten tissue (ii) shrink tissue and/or (iii) contour or sculpt the body.

When the plasma generator 100, 200, 300, 600, 800 is activated, in step 906, the electrosurgical generator applies a waveform including a predetermined power curve to the electrode of the plasma generator 100, 200, 300, 600, 800. In one embodiment, the predetermined power curve is configured such that electrosurgical energy is provided in a pulsed manner, with each pulse having a predetermined time duration and with the electrosurgical generator outputting a predetermined output power when the waveform is applied. The predetermined time duration of each pulse is selected such to be long enough to deliver enough energy to heat tissue to the desired temperature range. For example, in one embodiment, the power curve is configured such that the predetermined the time duration of a pulse is between 0.04 seconds and 0.08 seconds and the predetermined output power is between 24 Watts and 32 Watts, however other values are contemplated to be within the scope of the present disclosure. It is to be appreciated that, in some embodiments, the predetermined output power of the electrosurgical generator is selected based on the actual energy delivered to the tissue by the applicator. In some embodiments, the generator may be configured to determine to how much energy is delivered to tissue by the applicator based on generator settings (e.g., how much power is being currently outputted by the generator).

Furthermore, when the plasma generator 100, 200, 300, 600, 800 is activated, in step 906, a gas source (e.g., integrated with the electrosurgical generator or separate from the generator) is configured to provide inert gas to the distal tip (e.g., tip 606, 608) of the plasma device 100, 200, 300, 600, 800 at a predetermined flow rate. In one embodiment, the inert gas used is helium and the predetermined flow rate is between 1 liter per minute and 5 liters per minute.

In step 910, the user moves the distal tip of the plasma device 100, 200, 300, 600, 800 through the tissue plane at a predetermined speed. In one embodiment, the predetermined speed is 1 centimeter per second. It is to be appreciated that, in the method 900, the predetermined power curve of the waveform, the predetermined flow rate of the inert gas, and the predetermined speed of the tip through the tissue plane are selected such that, when steps 906-910 are performed, the temperature of the tissue being heated by the plasma emitted from the plasma device reaches at least 85° C. and the tissue is not heated in bulk (e.g., in areas surrounding or further away from the target tissue). but instead is heated instantaneously and cools quickly after treatment. After the desired effects are achieved, the plasma generator is removed, and the entry incision is closed, in step 908.

Unlike with bulk tissue heating, the rapid heating of tissue performed by the system of the present disclosure allows the tissue surrounding the treatment site to remain at much cooler temperatures resulting in rapid cooling after the application of the energy through conductive heat transfer. Additionally, the energy provided to the tissue using the electrosurgical apparatuses of the present disclosure is focused on heating the fibroseptal network (FSN) instead of the dermis. The majority of soft tissue contraction induced by subcutaneous energy delivery devices is due to its effect on the fibroseptal network. Because of these unique heating and cooling properties of the electrosurgical apparatuses of the present disclosure, immediate soft tissue contraction can be achieved without unnecessarily heating the full thickness of the dermis.

As discussed above, RF energy flows through the conductive plasma beam generated by the plasma generator or electrosurgical apparatus (e.g., apparatus 600, 800). This conductive plasma beam can be thought of as a flexible wire or electrode that “connects” to the tissue that represents the path of least resistance for the flow of the RF energy. The tissue that represents the path of least resistance is typically either the tissue that is in closest proximity to the tip of the plasma generator (e.g., tissue proximately disposed to ports 630 of tip 606 or 830 of tip 806) or the tissue that has the lowest impedance, i.e., tissue that has the lowest impedance relative to adjacent tissue. This means that, when an electrosurgical apparatus, such as apparatus 600 or 800, is used for the coagulation of subcutaneous soft tissue, the energy from the ports 630, 830 of the plasma generator or apparatus 600, 800 is not directed or focused in any set direction when activated in the subdermal plane as in some RFAL devices. Instead, the energy provided via ports 630, 830 finds the tissue that represents the path of least resistance surrounding the tip 606, 806 of the plasma generator or device. In other words, the energy from the tip of the plasma generator may be directed in a radial direction (relative to the shaft 604 of the plasma generator 600 or shaft 804 of generator 800) from the tip 606, 806, above the tip 606, 806, below the tip 606, 806, adjacent either side of the tip 606, 806 and anywhere in between effectively providing energy in 360° about the tip 606, 806.

If the path of least resistance is through the overlying dermis, the plasma energy will be directed to the dermis. If the path of least resistance is through the fibroseptal network, the plasma energy will be directed there. As the tip of the plasma generator 600, 800 is drawn through the subdermal plane, new structures are introduced to the tip 606 of the device 600 or tip 806 of device 800 and the path of least resistance is constantly changing. As the energy is constantly finding a new preferred path, the plasma beam quickly alternates between treating the different tissue surrounding the tip 606 of the device 600 or tip 806 of device 800. This allows for 360° tissue treatment without the need for the user to redirect the flow of energy.

Since the FSN is typically the closest tissue to the tip of the plasma generator 100, 200, 300, 600, 800 the vast majority of the energy delivered by the device results in coagulation and contraction of the fibroseptal bands. Maximizing the energy flow to the FSN expedites the soft tissue contraction process.

However, it is to be appreciated that not all RF is created equal. Very different tissue effects can result at the same power setting by simply changing from a waveform designed for cutting to a waveform designed for coagulation. The RF waveform of the plasma generator 100, 200, 300, 600, 800 has lower current than other RF devices. In most cases, the current of the plasma generator 100, 200, 300, 600, 800 is an order of magnitude lower. Exemplary waveforms are shown and described in commonly-owned PCT Patent Application No. PCT/US2017/062195 filed Nov. 17, 2017 entitled “ELECTROSURGICAL APPARATUS WITH DYNAMIC LEAKAGE CURRENT COMPENSATION AND DYNAMIC RF MODULATION” and PCT Patent Application No. PCT/US2018/015948 filed Jan. 30, 2018 entitled “ELECTROSURGICAL APPARATUS WITH FLEXIBLE SHAFT”, the entire contents of both of which is hereby incorporated by reference.

The current of the plasma generator waveform flows through the conductive plasma beam to create additional beneficial Joule heating of the target tissue. However, since the current is so low, it is dispersed before it is able to penetrate deep into the tissue. This allows for soft tissue heating with minimal depth of thermal effect. This also prevents tissue from being overtreated when subjected to multiple treatment passes. Previously treated tissue has higher impedance. As tissue is treated, it coagulates and desiccates resulting in an increase in tissue impedance. Low current cannot push through the higher impedance tissue. As the plasma generator 100, 200, 300, 600, 800 passes in proximity to previously treated tissue, the energy will follow the path of least resistance (lower impedance) and preferentially treat previously untreated tissue. This prevents overtreating any one particular area with multiple passes and maximizes the treatment of untreated tissue.

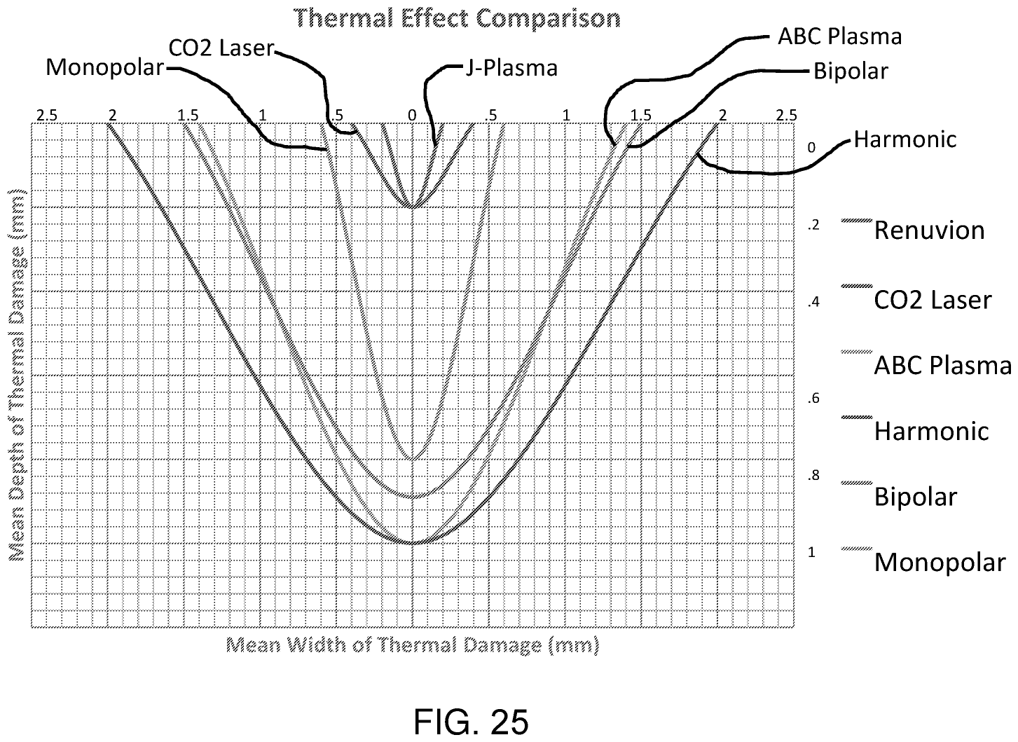

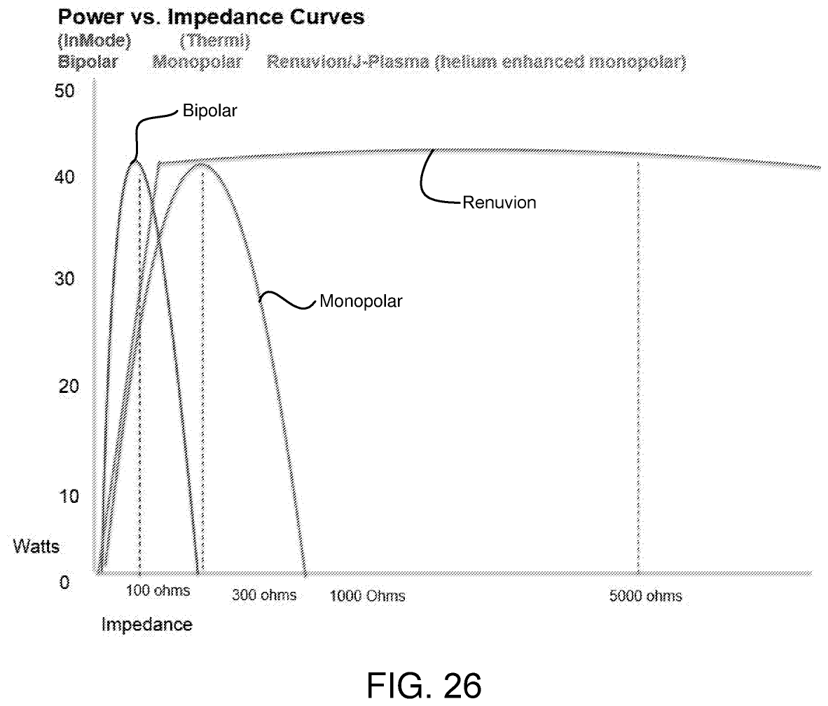

The design of the electrosurgical generator for use with the plasma generators 100, 200, 300, 600, 800 of the present disclosure is fundamentally different from monopolar and bipolar devices. In one embodiment, the electrosurgical generator applies power based on impedance determined at the output of the electrosurgical generator. As shown in the in FIG. 25, monopolar and bipolar devices have limited power output in tissues with higher impedance, such as fat. Electrosurgical generators coupled to such monopolar and bipolar devices are programmed, e.g., hardwired or software-based, to follow the curves illustrated in FIG. 25. The plasma generator of the present disclosure is configured to maintain consistent power output over a wide range of impedances, as shown in FIG. 25 by the curve labeled Renuvion. For example, the plasma generators of the present disclosure apply a constant or predetermined output power level, e.g., approximately 40 watts, over a range of tissue impedances, e.g., 150 ohms to at least 5000 ohms. When used for the coagulation and tightening of subdermal tissue, the plasma generators of the present disclosure are not self-limiting and will provide unencumbered delivery of power regardless of the tissue impedance.

The plasma generators 100, 200, 300, 600, 800 of the present disclosure achieve soft tissue coagulation and contraction by heating tissue for very short periods of time followed by immediate cooling. This allows for immediate coagulation and contraction of the tissue with very limited depth of thermal effect, as compared to other surgical devices as shown in FIG. 24. Since the plasma generators 100, 200, 300, 600, 800 of the present disclosure work on the scientific principle of the path of least resistance, the vast majority of the energy from the device results in coagulation and contraction of the FSN which is the tissue in closest proximity to the tip of the device. The plasma generators 100, 200, 300, 600, 800 of the present disclosure focuses delivery of energy on immediate heating of the FSN which results in immediate soft tissue contraction without unnecessarily heating the full thickness of the dermis.

The plasma generators 100, 200, 300, 600, 800 of the present disclosure include several features that result in a unique and effective method of action for subdermal coagulation and contraction of soft tissue. As described above, these features include a plasma generator and system configured: (1) to achieve soft tissue coagulation and contraction by rapidly heating the treatment site to temperatures greater than 85° C. for between 0.040 and 0.080 seconds; (2) such that the tissue surrounding the treatment site remains at much cooler temperatures resulting in rapid cooling after the application of the energy through conductive heat transfer; (3) such that focused delivery of energy occurs on immediate heating of the FSN resulting in immediate soft tissue contraction without unnecessarily heating the full thickness of the dermis; (4) to provide 360° tissue treatment without the need for the user to redirect the flow of energy due to electrical energy taking the path of least resistance; (5) to deliver unencumbered power regardless of the tissue impedance due to the unique power output from the electrosurgical generator; and (6) to output low current RF energy resulting in minimal depth of thermal effect and prevention of over-treating tissue when performing multiple passes

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. An electrosurgical apparatus comprising:
a housing;
a shaft extending from the housing and disposed along a longitudinal axis;
an electrically conducting member;
a distal tip including an interior, an outer wall, and at least one port, the at least one port disposed through the outer wall and oriented in a radial direction relative to the longitudinal axis, the electrically conducting member at least partially disposed in the interior of the distal tip and configured to energize inert gas provided via the shaft to the interior of the distal tip such that plasma is ejected from the at least one port.

2. The electrosurgical apparatus of claim 1, wherein the at least one port is configured such that the distal tip has a 180-degree tissue treatment area about the longitudinal axis.

3. The electrosurgical apparatus of claim 1, wherein the interior of the distal tip includes an inner wall that is slanted with respect to the longitudinal axis and is configured to direct the plasma generated by the electrosurgical apparatus and the inert gas provided to the distal tip through the at least one port to the exterior of the electrosurgical apparatus.

4. The electrosurgical apparatus of claim 1, wherein the distal tip includes at least one second port disposed through the outer wall of the distal tip and oriented in a radial direction to the longitudinal axis, the at least one second port diametrically opposed from the at least one first port.

5. The electrosurgical apparatus of claim 4, wherein the interior of the distal tip includes an inner wall having a first portion and a second portion, the first portion is slanted with respect to the longitudinal axis and is configured to direct the plasma generated by the electrosurgical apparatus and the inert gas provided to the distal tip through the at least one first port to the exterior of the electrosurgical apparatus, the second portion is slanted with respect to the longitudinal axis and is configured to direct the plasma generated by the electrosurgical apparatus and the inert gas provided to the distal tip through the at least one second portion to the exterior of the electrosurgical apparatus.

6. The electrosurgical apparatus of claim 4, wherein the at least one first port and at least one second port are configured such that the distal tip has a 360-degree tissue treatment area about the longitudinal axis.

7. The electrosurgical apparatus of claim 1, further comprising a support tube having a proximal and a distal end, wherein the proximal end of the support tube is disposed through a distal end of the shaft and coupled to the interior of the shaft and the distal end of the support tube is disposed through a proximal end of the distal tip and coupled to the interior of the distal tip, the support tube configured to couple the distal tip to the distal end of the shaft and to provide support to the coupling of the distal tip to the distal end of the shaft.

8. The electrosurgical apparatus of claim 7, wherein the support tube is made of a non-conducting material.

9. The electrosurgical apparatus of claim 7, wherein the support tube is coupled the shaft and distal tip via an adhesive.

10. The electrosurgical apparatus of claim 1, wherein the electrically conducting member is a support tube having a proximal and a distal end, wherein the proximal end of the support tube is disposed through a distal end of the shaft and coupled to the interior of the shaft and the distal end of the support tube is disposed through a proximal end of the distal tip and coupled to the interior of the distal tip, the support tube configured couple the distal tip to the distal end of the shaft and to provide support to the coupling of the distal tip to the distal end of the shaft.

11. The electrosurgical apparatus of claim 1, further comprising a coupling member disposed between the shaft and the distal tip, the coupling member configured to couple the distal tip to the shaft.

12. The electrosurgical apparatus of claim 11, further comprising a support tube having a proximal and a distal end, wherein the proximal end of the support tube is disposed through a distal end of the shaft and coupled to the interior of the shaft, the distal end of the support tube is disposed through a proximal end of the distal tip and coupled to the interior of the distal tip, and the coupling member is formed via injection molding between the distal end of the shaft and the proximal end of the distal tip over the support tube.

13. The electrosurgical apparatus of claim 12, wherein the support tube is coupled the shaft and distal tip via an adhesive.

14. The electrosurgical apparatus of claim 1, wherein the interior of the distal tip includes a slot that receives a distal end of the electrically conducting member.

15. The electrosurgical apparatus of claim 14, wherein the electrically conducting member includes a bent distal end disposed in the slot, the bent distal end configured to prevent distal tip from being decoupled from the shaft.

16. The electrosurgical apparatus of claim 1, wherein the distal tip includes a cap that is formed via injection molding over a distal end of the electrically conducting member to prevent the distal tip from being decoupled from the shaft.

17. The electrosurgical apparatus of claim 1, wherein the distal tip is formed via injection molding over a distal end of the electrically conducting member to prevent the distal tip from being decoupled from the shaft.

18. The electrosurgical apparatus of claim 1, wherein the distal tip includes at least one protrusion and a distal end of the shaft includes at least one slot configured to receive the protrusion such that the distal tip is securely coupled to the distal end of the shaft.

19. The electrosurgical apparatus of claim 18, wherein the at least one slot includes a first portion aligned along the longitudinal axis and a second portion extending perpendicularly to the longitudinal axis.

20. The electrosurgical apparatus of claim 1, further comprising a connector and a cable having a first end and a second end, the first end of the cable coupled to the housing and the second end of the cable coupled to the connector, the connector configured to be coupled to an electrosurgical generator to receive electrosurgical energy and the inert gas to be provided to the housing via the cable.

21. The electrosurgical apparatus of claim 20, further comprising a stranded wire that couples the electrically conducting member to the cable, the stranded wire is configured to provide electrosurgical energy to the electrically conducting member.

22. The electrosurgical apparatus of claim 1, wherein the shaft includes at least one marking disposed a predetermined distance from one of a distal end of the distal tip or a center of the at least one port, such that when the at least one marking becomes visible to a user as the distal tip and shaft are pulled from patient tissue, the user is alerted to deactivate the electrosurgical apparatus.

23. A method for using a plasma device to tighten tissue, the method comprising:
creating an incision through tissue to access a subdermal tissue plane;
inserting the plasma device into the subdermal tissue plane;
activating the plasma device to generate and apply plasma to the subdermal tissue plane;
moving the plasma device through the subdermal tissue plane; and
heating tissue in the subdermal tissue plane to a predetermined temperature to tighten the tissue.

24. The method of claim 23, wherein a waveform including a predetermined power curve is applied to an electrode of the plasma device when the plasma device is activated.

25. The method of claim 24, wherein the predetermined power curve is configured such that the power applied to the electrode is between 24 and 32 Watts.

26. The method of claim 24, wherein the predetermined power curve is configured such that the generated plasma is pulsed.

27. The method of claim 26, wherein each pulse of the pulsed plasma includes a predetermined time duration.

28. The method of claim 27, wherein the predetermined time duration is between 0.04 and 0.08 seconds.

29. The method of claim 23, wherein inert gas is provided at a predetermined flow rate when the plasma device is activated.

30. The method of claim 29, wherein the predetermined flow rate is between 1.5 liters per minute to 3 liters per minute.

31. The method of claim 29, wherein the inert gas is helium.

32. The method of claim 23, wherein the predetermined temperature is approximately 85 Celsius.

33. The method of claim 23, wherein a distal tip of the plasma device is moved through the subdermal tissue plane at a predetermined speed.

34. The method of claim 33, wherein the predetermined speed is 1 centimeter per second.

35. The method of claim 23, further comprising:
removing the plasma device from the subdermal tissue plane; and
closing the entry incision.
